SEQUENCE LISTING (182 sequences)

SEQ ID NO: 1 - protein, 6 aa, Artificial Sequence
  CDRa1 consensus sequence
  MISC_FEATURE (6)..(6): any amino acid
DRGSQX

SEQ ID NO: 2 - protein, 6 aa, Artificial Sequence
  CDRa2 consensus sequence
  MISC_FEATURE (3)..(3): wherein Xaa is any amino acid, preferably S or Q, more preferably Q
  MISC_FEATURE (4)..(4): Xaa is any amino acid, preferably T, E, D or A, more preferably E,
IYXXGD

SEQ ID NO: 3 - protein, 15 aa, Artificial Sequence
  CDRa3 consensus sequence
  MISC_FEATURE (6)..(6): wherein Xaa is any amino acid, preferably, X4 is  S, N, D, P, T, or E, more preferably P.
  MISC_FEATURE (8)..(8): wherein Xaa is any amino acid, preferably, Xaa is L, I, V, Q, N, P, S, A, G, T, D, K, R, F, more preferably V.
  MISC_FEATURE (9)..(9): wherein Xaa is any amino acid, preferably, Xaa is E, A, V, R, N, G, Y, I, D, H, L, P, S, Q, more preferably V
  MISC_FEATURE (12)..(12): wherein Xaa is any amino acid, preferably, Xaa is is S, I, Q, A, R, L, P, Y, K, M, more preferably Q
CAAVIXNXXGGXLTF

SEQ ID NO: 4 - protein, 5 aa, Artificial Sequence
  CDRb1
  MISC_FEATURE (1)..(1): wherein Xaa is any amino acid, preferably S or P, more preferably P
  MISC_FEATURE (5)..(5): wherein Xaa is any amino acid, preferably S or A, more preferably A
XGHRX

SEQ ID NO: 5 - protein, 6 aa, Artificial Sequence
  CDRb2 consensus sequence
  MISC_FEATURE (2)..(2): wherein Xaa is any amino acid, preferably Xaa is V, I, F or E, more preferably I or V, more preferably V
  MISC_FEATURE (3)..(3): wherein Xaa is any amino acid, preferably Xaa is H, D, or S, more preferably D or H, more preferably H
  MISC_FEATURE (4)..(4): wherein Xaa is any amino acid, preferably G, R or E, more preferably G
  MISC_FEATURE (5)..(5): wherein Xaa is any amino acid, preferably Q, R, A, F, V, Q, E, L, Y or T, more preferably E or A, more preferably E
  MISC_FEATURE (6)..(6): wherein Xaa is any amino acid, preferably is E, A or Q, more preferably E or Q, more preferably E
YXXXXX

SEQ ID NO: 6 - protein, 14 aa, Artificial Sequence
  CDRb3 consensus sequence
  MISC_FEATURE (11)..(11): wherein Xaa is any amino acid preferably V
CASSPWDSPNXQYF

SEQ ID NO: 7 - protein, 509 aa, Artificial Sequence
  full length PRAME protein, P78395
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 8 - protein, 9 aa, Artificial Sequence
  PRAME-004 peptide
SLLQHLIGL

SEQ ID NO: 9 - protein, 6 aa, Artificial Sequence
  CDRa2 (i.e. R16P1C10)
IYSNGD

SEQ ID NO: 10 - protein, 15 aa, Artificial Sequence
  CDRa3 (i.e R16P1C10)
CAAVISNFGNEKLTF

SEQ ID NO: 11 - protein, 6 aa, Artificial Sequence
  CDRa1 (i.e HiAff1)
DRGSQS

SEQ ID NO: 12 - protein, 5 aa, Artificial Sequence
  CDRb1 (i.e R16P1C10)
SGHRS

SEQ ID NO: 13 - protein, 14 aa, Artificial Sequence
  CDRb3 (i.e R16P1C10)
CASSPWDSPNEQYF

SEQ ID NO: 14 - protein, 6 aa, Artificial Sequence
  CDRb2 (i.e R16P1C10)
YFSETQ

SEQ ID NO: 15 - protein, 6 aa, Artificial Sequence
  CDRb2 second consensus sequence
  MISC_FEATURE (5)..(5): wherein Xaa is any amino acid, preferably Q, R, A, F, V, Q, E, L, Y or T, preferably E
YVHGXE

SEQ ID NO: 16 - protein, 6 aa, Artificial Sequence
  CDRa1 variant 1
DRGSQL

SEQ ID NO: 17 - protein, 6 aa, Artificial Sequence
  CDRa2 variant 2
IYQEGD

SEQ ID NO: 18 - protein, 15 aa, Artificial Sequence
  CDRa3
CAAVINNPSGGMLTF

SEQ ID NO: 19 - protein, 15 aa, Artificial Sequence
  CDRa3
CAAVIDNSNGGILTF

SEQ ID NO: 20 - protein, 15 aa, Artificial Sequence
  CDRa3
CAAVIDNPSGGILTF

SEQ ID NO: 21 - protein, 15 aa, Artificial Sequence
  CDRa3
CAAVIDNDQGGILTF

SEQ ID NO: 22 - protein, 15 aa, Artificial Sequence
  CDRa3
CAAVIPNPPGGKLTF

SEQ ID NO: 23 - protein, 15 aa, Artificial Sequence
  CDRa3
CAAVIPNPGGGALTF

SEQ ID NO: 24 - protein, 15 aa, Artificial Sequence
  CDRa3
CAAVIPNSAGGRLTF

SEQ ID NO: 25 - protein, 15 aa, Artificial Sequence
  CDRa3
CAAVIPNLEGGSLTF

SEQ ID NO: 26 - protein, 15 aa, Artificial Sequence
  CDRa3
CAAVIPNRLGGYLTF

SEQ ID NO: 27 - protein, 15 aa, Artificial Sequence
  CDRa3
CAAVIPNTDGGRLTF

SEQ ID NO: 28 - protein, 15 aa, Artificial Sequence
  CDRa3
CAAVIPNQRGGALTF

SEQ ID NO: 29 - protein, 15 aa, Artificial Sequence
  CDRa3
CAAVIPNVVGGILTF

SEQ ID NO: 30 - protein, 15 aa, Artificial Sequence
  CDRa3
CAAVITNIAGGSLTF

SEQ ID NO: 31 - protein, 15 aa, Artificial Sequence
  CDRa3
CAAVIPNNDGGYLTF

SEQ ID NO: 32 - protein, 15 aa, Artificial Sequence
  CDRa3
CAAVIPNGRGGLLTF

SEQ ID NO: 33 - protein, 15 aa, Artificial Sequence
  CDRa3
CAAVIPNTHGGPLTF

SEQ ID NO: 34 - protein, 15 aa, Artificial Sequence
  CDRa3
CAAVIPNDVGGSLTF

SEQ ID NO: 35 - protein, 15 aa, Artificial Sequence
  CDRa3
CAAVIENKPGGPLTF

SEQ ID NO: 36 - protein, 15 aa, Artificial Sequence
  CDRa3
CAAVIDNPVGGPLTF

SEQ ID NO: 37 - protein, 15 aa, Artificial Sequence
  CDRa3
CAAVIPNNNGGALTF

SEQ ID NO: 38 - protein, 15 aa, Artificial Sequence
  CDRa3
CAAVIPNDQGGILTF

SEQ ID NO: 39 - protein, 15 aa, Artificial Sequence
  CDRa3
CAAVIPNVVGGQLTF

SEQ ID NO: 40 - protein, 15 aa, Artificial Sequence
  CDRa3
CAAVIPNSYGGLLTF

SEQ ID NO: 41 - protein, 15 aa, Artificial Sequence
  CDRa3
CAAVIPNDDGGLLTF

SEQ ID NO: 42 - protein, 15 aa, Artificial Sequence
  CDRa3
CAAVIPNAAGGLLTF

SEQ ID NO: 43 - protein, 15 aa, Artificial Sequence
  CDRa3
CAAVIPNTIGGLLTF

SEQ ID NO: 44 - protein, 15 aa, Artificial Sequence
  CDRa3
CAAVIPNTRGGLLTF

SEQ ID NO: 45 - protein, 5 aa, Artificial Sequence
  CDRb1
PGHRA

SEQ ID NO: 46 - protein, 6 aa, Artificial Sequence
  CDRb2 (i.e HiAff1)
YVHGEE

SEQ ID NO: 47 - protein, 6 aa, Artificial Sequence
  CDRB2 (I.E IMPROVED)
YVHGAE

SEQ ID NO: 48 - protein, 14 aa, Artificial Sequence
  CDRb3 (i.e HiAff1)
CASSPWDSPNVQYF

SEQ ID NO: 49 - protein, 5 aa, Artificial Sequence
  CDRb1 (i.e Improved)
PGHRS

SEQ ID NO: 50 - protein, 9 aa, Artificial Sequence
  IFIT1-001
VLLHHQIGL

SEQ ID NO: 51 - protein, 9 aa, Artificial Sequence
  IFT17-003
FMNPHLISV

SEQ ID NO: 52 - protein, 9 aa, Artificial Sequence
  FADS2-001
LLLAHIIAL

SEQ ID NO: 53 - protein, 9 aa, Artificial Sequence
  CTBP1-001
ALMYHTITL

SEQ ID NO: 54 - protein, 26 aa, Artificial Sequence
  FR1-a
QKEVEQNSGPLSVPEGAIASLNCTYS

SEQ ID NO: 55 - protein, 17 aa, Artificial Sequence
  FR2-a
FFWYRQYSGKSPELIMS

SEQ ID NO: 56 - protein, 33 aa, Artificial Sequence
  FR3-a
KEDGRFTAQLNKASQYVSLLIRDSQPSDSATYL

SEQ ID NO: 57 - protein, 14 aa, Artificial Sequence
  FR4-a
GTGTRLTIIPNIQN

SEQ ID NO: 58 - protein, 26 aa, Artificial Sequence
  FR1-b
KAGVTQTPRYLIKTRGQQVTLSCSPI

SEQ ID NO: 59 - protein, 17 aa, Artificial Sequence
  FR2-b
VSWYQQTPGQGLQFLFE

SEQ ID NO: 60 - protein, 36 aa, Artificial Sequence
  FR3-b
RNKGNFPGRFSGRQFSNSSSEMNISNLELGDSALYL

SEQ ID NO: 61 - protein, 36 aa, Artificial Sequence
  FR3-b
RNKGNFPGRFSGRQFSNFSSEMNISNLELGDSALYL

SEQ ID NO: 62 - protein, 14 aa, Artificial Sequence
  FR4-b
GPGTRLTVTEDLKN

SEQ ID NO: 63 - protein, 117 aa, Artificial Sequence
  V3 of Fc-containing HiAff1 TCR/mAb diabody
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIDNDQGGILTFGTGTRLTIIPNIQN

SEQ ID NO: 64 - protein, 8 aa, Artificial Sequence
  L1 in the molecule Fc-containing HiAff1 TCR/mAb diabody
GGGSGGGG

SEQ ID NO: 65 - protein, 107 aa, Artificial Sequence
  VL of the humanized UCHT1
DIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIK

SEQ ID NO: 66 - protein, 229 aa, Artificial Sequence
  Fc1 with “Knob” mutation
EPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 67 - protein, 118 aa, Artificial Sequence
  vbeta variable domain (B2)
KAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKN

SEQ ID NO: 68 - protein, 229 aa, Artificial Sequence
  Fc2 with “Hole mutation”
EPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 69 - protein, 5 aa, Artificial Sequence
  Linker
TVAAP

SEQ ID NO: 70 - protein, 4 aa, Artificial Sequence
  Linker
GGGS

SEQ ID NO: 71 - protein, 5 aa, Artificial Sequence
  Linker
GGGGS

SEQ ID NO: 72 - protein, 5 aa, Artificial Sequence
  Linker
TVLRT

SEQ ID NO: 73 - protein, 6 aa, Artificial Sequence
  Linker
TVSSAS

SEQ ID NO: 74 - protein, 10 aa, Artificial Sequence
  Linker
GGGGSGGGGS

SEQ ID NO: 75 - protein, 8 aa, Artificial Sequence
  Linker
GGGGSAAA

SEQ ID NO: 76 - protein, 15 aa, Artificial Sequence
  Linker
GGGGSGGGGSGGGGS

SEQ ID NO: 77 - protein, 27 aa, Artificial Sequence
  Linker
GGGGSGGGGSGGGGSGGGGSGGGGSGS

SEQ ID NO: 78 - protein, 7 aa, Artificial Sequence
  Linker
TVLSSAS

SEQ ID NO: 79 - protein, 232 aa, Artificial Sequence
  native Fc  (IGHG1*01)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 119 aa, Artificial Sequence
  hinge-CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAG

SEQ ID NO: 81 - protein, 503 aa, Artificial Sequence
  first polypeptide of the scTCR-Fab construct
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGQKEVEQNSGPLSVPEGAIASLNCTYSDRGSQSFFWYRQYSGKSPELIMSIYQEGDKEDGRFTAQLNKASQYVSLLIRDSQPSDSATYLCAAVIDNDQGGILTFGTGTRLTIIPNIQNGGGGSGGGGSGGGGSGGGGSGGGGSGSKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKN

SEQ ID NO: 82 - protein, 214 aa, Artificial Sequence
  second polypeptide of the scTCR-Fab
DIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 118 aa, Artificial Sequence; identical to SEQ ID NO: 67
  Vbeta-domain (designated B2)

SEQ ID NO: 84 - protein, 18 aa, Artificial Sequence
  L-Myc tag
AAAGGSGGEQKLISEEDL

SEQ ID NO: 85 - protein, 117 aa, Artificial Sequence
  Valpha A_HiAff1#1
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNPGGGALTFGTGTRLTIIPNIQN

SEQ ID NO: 86 - protein, 117 aa, Artificial Sequence
  Valpha A_HiAff1#2
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNSAGGRLTFGTGTRLTIIPNIQN

SEQ ID NO: 87 - protein, 117 aa, Artificial Sequence
  Valpha A_HiAff1#3
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNLEGGSLTFGTGTRLTIIPNIQN

SEQ ID NO: 88 - protein, 117 aa, Artificial Sequence
  Valpha A_HiAff1#4
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNRLGGYLTFGTGTRLTIIPNIQN

SEQ ID NO: 89 - protein, 117 aa, Artificial Sequence
  Valpha A_HiAff1#5
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNTDGGRLTFGTGTRLTIIPNIQN

SEQ ID NO: 90 - protein, 117 aa, Artificial Sequence
  Valpha A_HiAff1#6
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNQRGGALTFGTGTRLTIIPNIQN

SEQ ID NO: 91 - protein, 117 aa, Artificial Sequence
  Valpha A_HiAff1#7
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNVVGGILTFGTGTRLTIIPNIQN

SEQ ID NO: 92 - protein, 117 aa, Artificial Sequence
  Valpha A_HiAff1#9
QKEVEQNSGPLSVPEGAIASLNCTYSDRGSQSFFWYRQYSGKSPELIMSIYQEGDKEDGRFTAQLNKASQYVSLLIRDSQPSDSATYLCAAVITNIAGGSLTFGTGTRLTIIPNIQN

SEQ ID NO: 93 - protein, 117 aa, Artificial Sequence
  Valpha A_HiAff1#10
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNDQGGYLTFGTGTRLTIIPNIQN

SEQ ID NO: 94 - protein, 117 aa, Artificial Sequence
  Valpha A_HiAff1#11
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNGRGGLLTFGTGTRLTIIPNIQN

SEQ ID NO: 95 - protein, 117 aa, Artificial Sequence
  Valpha A_HiAff1#12
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNTHGGPLTFGTGTRLTIIPNIQN

SEQ ID NO: 96 - protein, 117 aa, Artificial Sequence
  Valpha A_HiAff1#13
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNDVGGSLTFGTGTRLTIIPNIQN

SEQ ID NO: 97 - protein, 117 aa, Artificial Sequence
  Valpha A_HiAff1#14
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIENKPGGPLTFGTGTRLTIIPNIQN

SEQ ID NO: 98 - protein, 117 aa, Artificial Sequence
  Valpha A_HiAff1#15
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIDNPVGGPLTFGTGTRLTIIPNIQN

SEQ ID NO: 99 - protein, 117 aa, Artificial Sequence
  Valpha A_HiAff1#16
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNNIGGALTFGTGTRLTIIPNIQN

SEQ ID NO: 100 - protein, 117 aa, Artificial Sequence
  Valpha A_HiAff1#17
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIPNDQGGILTFGTGTRLTIIPNIQN

SEQ ID NO: 101 - protein, 117 aa, Artificial Sequence
  Valpha A_HiAff1#18
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIPNDQGGILTFGTGTRLTIIPNIQN

SEQ ID NO: 102 - protein, 117 aa, Artificial Sequence
  Valpha A_HiAff1#19
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIPNSAGGRLTFGTGTRLTIIPNIQN

SEQ ID NO: 103 - protein, 117 aa, Artificial Sequence
  Valpha A_HiAff1#20
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIPNRLGGYLTFGTGTRLTIIPNIQN

SEQ ID NO: 104 - protein, 117 aa, Artificial Sequence
  Valpha A_HiAff1#22
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIPNTHGGPLTFGTGTRLTIIPNIQN

SEQ ID NO: 105 - protein, 117 aa, Artificial Sequence
  Valpha A_HiAff1#23
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIPNVVGGQLTFGTGTRLTIIPNIQN

SEQ ID NO: 106 - protein, 117 aa, Artificial Sequence
  Valpha A_HiAff1#24
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIPNSYGGLLTFGTGTRLTIIPNIQN

SEQ ID NO: 107 - protein, 117 aa, Artificial Sequence
  Valpha A_HiAff1#26
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIPNAAGGLLTFGTGTRLTIIPNIQN

SEQ ID NO: 108 - protein, 117 aa, Artificial Sequence
  Valpha A_HiAff1#27
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIPNTIGGLLTFGTGTRLTIIPNIQN

SEQ ID NO: 109 - protein, 117 aa, Artificial Sequence
  Valpha A_HiAff1#29
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIPNDDGGRLTFGTGTRLTIIPNIQN

SEQ ID NO: 110 - protein, 117 aa, Artificial Sequence
  Valpha A_HiAff1#30
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIPNTRGGLLTFGTGTRLTIIPNIQN

SEQ ID NO: 111 - protein, 117 aa, Artificial Sequence
  Valpha A_HiAff1_38
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIDNDQGGILTFGTGTRLTIIPNIQN

SEQ ID NO: 112 - protein, 118 aa, Artificial Sequence
  Vbeta B2_S85F
KAGVTQTPRYLIKTRGQQVTLSCSPIPGHRAVSWYQQTPGQGLQFLFEYVHGEERNKGNFPGRFSGRQFSNFSSEMNISNLELGDSALYLCASSPWDSPNVQYFGPGTRLTVTEDLKN

SEQ ID NO: 113 - protein, 117 aa, Artificial Sequence; identical to SEQ ID NO: 106
  Valpha A_HiAff1#24

SEQ ID NO: 114 - protein, 117 aa, Artificial Sequence; identical to SEQ ID NO: 101
  Valpha A_HiAff1#18

SEQ ID NO: 115 - protein, 117 aa, Artificial Sequence; identical to SEQ ID NO: 105
  Valpha A_HiAff1#23

SEQ ID NO: 116 - protein, 117 aa, Artificial Sequence; identical to SEQ ID NO: 103
  Valpha A_HiAff1#20

SEQ ID NO: 117 - protein, 20 aa, Artificial Sequence
  Linker
GGGGSGGGGSGGGGSGGGGS

SEQ ID NO: 118 - protein, 117 aa, Artificial Sequence; identical to SEQ ID NO: 111
  Valpha A_HiAff1_38

SEQ ID NO: 119 - protein, 461 aa, Artificial Sequence
  first polypeptide of Fc-containing HiAff1 bispecific TCR/mAb(anti-CD3) diabody
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIDNDQGGILTFGTGTRLTIIPNIQNGGGSGGGGDIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 120 - protein, 477 aa, Artificial Sequence
  second polypeptide of Fc-containing HiAff1 bispecific TCR/mAb(anti-CD3) diabody
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSSGGGSGGGGKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 121 - protein, 108 aa, Artificial Sequence
  VH of anti-CD3
DIQMTQSPSSLSASVGDRVTITCRASQDIRNYLNWYQQKPGKAPKLLIYYTSRLHSGVPSRFSGSGSGTDYTLTISSLQPEDIATYFCQQGQTLPWTFGQGTKVEIKR

SEQ ID NO: 122 - protein, 108 aa, Artificial Sequence; identical to SEQ ID NO: 121
  VL of anti-CD3

SEQ ID NO: 123 - protein, 106 aa, Artificial Sequence
  CL
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 262 aa, Artificial Sequence
  alpha beta fusion
QKEVEQNSGPLSVPEGAIASLNCTYSDRGSQSFFWYRQYSGKSPELIMSIYQEGDKEDGRFTAQLNKASQYVSLLIRDSQPSDSATYLCAAVIDNDQGGILTFGTGTRLTIIPNIQNGGGGSGGGGSGGGGSGGGGSGGGGSGSKAGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKN

SEQ ID NO: 125 - protein, 9 aa, Artificial Sequence
  Peptide ATP1A1-001
FLPIHLLGL

SEQ ID NO: 126 - protein, 9 aa, Artificial Sequence
  Peptide NADK-002
YLDGHLITT

SEQ ID NO: 127 - protein, 9 aa, Artificial Sequence
  Peptide ITSN1-001
ILAMHLIDV

SEQ ID NO: 128 - protein, 9 aa, Artificial Sequence
  Peptide SMA-003
IVINHVISV

SEQ ID NO: 129 - protein, 9 aa, Artificial Sequence
  Peptide SF3B3-005
TLVYHVVGV

SEQ ID NO: 130 - protein, 9 aa, Artificial Sequence
  Peptide SF3B3-005
TLVYHVVGV

SEQ ID NO: 131 - protein, 9 aa, Artificial Sequence
  Peptide NCAM2-001
FVQPHIIQL

SEQ ID NO: 132 - protein, 9 aa, Artificial Sequence
  Peptide KNT-001
YMLEHVITL

SEQ ID NO: 133 - protein, 9 aa, Artificial Sequence
  Peptide EHD4-001
ALAKHLIKI

SEQ ID NO: 134 - protein, 9 aa, Artificial Sequence
  Peptide EHD-001
ALANHLIKV

SEQ ID NO: 135 - protein, 9 aa, Artificial Sequence
  Peptide MCMB-002
YLILHLIST

SEQ ID NO: 136 - protein, 9 aa, Artificial Sequence
  Peptide HSPA5-001
RVMEHFIKL

SEQ ID NO: 137 - protein, 9 aa, Artificial Sequence
  Peptide CEBPZ-002
KLYQHEINL

SEQ ID NO: 138 - protein, 9 aa, Artificial Sequence
  DCAF12-001
SFYEHIITV

SEQ ID NO: 139 - protein, 9 aa, Artificial Sequence
  Peptide EHD2-002
ALASHLIEA

SEQ ID NO: 140 - protein, 9 aa, Artificial Sequence
  Peptide KIAA1324-001
SLADRLIGV

SEQ ID NO: 141 - protein, 9 aa, Artificial Sequence
  Peptide MYO1A-001
LVSEHVIEL

SEQ ID NO: 142 - protein, 9 aa, Artificial Sequence
  Peptide SERPINA6-001
SLYKHLVAL

SEQ ID NO: 143 - protein, 9 aa, Artificial Sequence
  Peptide SMARCD1-001
IIINHVISV

SEQ ID NO: 144 - protein, 9 aa, Artificial Sequence
  Peptide TSC1-001
SLLGHVIRL

SEQ ID NO: 145 - protein, 9 aa, Artificial Sequence
  Peptide UGT-005
SLIEHMIML

SEQ ID NO: 146 - protein, 9 aa, Artificial Sequence
  Peptide VAV1-001
NLLPHAINL

SEQ ID NO: 147 - protein, 9 aa, Artificial Sequence
  Peptide WDFY3-004
SLFEHFIEL

SEQ ID NO: 148 - protein, 134 aa, Artificial Sequence
  R16P1C10 alpha variable domain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAVISNFGNEKLTFGTGTRLTIIP

SEQ ID NO: 149 - protein, 132 aa, Artificial Sequence
  R16P1C10 beta variable domain
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPWDSPNEQYFGPGTRLTVT

SEQ ID NO: 150 - protein, 258 aa, Artificial Sequence
  scTCR CDR6
QKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNIQNGGGGSGGGGSGGGGSGGGGSGGGGSGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKN

SEQ ID NO: 151 - protein, 25 aa, Artificial Sequence
  linker sequence
GGGGSGGGGSGGGGSGGGGSGGGGS

SEQ ID NO: 152 - protein, 87 aa, Artificial Sequence
  Aga2p yeast mating protein together with its leader sequence
MQLLRCFSIFSVIASVLAQELTTICEQIPSPTLESTPYSLSTTTILANGKAMQGVFEYYKSVTFVSNCGSHPSTTSKGSPINTQYVF

SEQ ID NO: 153 - protein, 381 aa, Artificial Sequence
  Leader peptide- Aga2p-FLAG -L- scTCR CDR6
MQLLRCFSIFSVIASVLAQELTTICEQIPSPTLESTPYSLSTTTILANGKAMQGVFEYYKSVTFVSNCGSHPSTTSKGSPINTQYVFGGGGSDYKDDDDKGGGASQKEVEQNSGPLSVPEGAIASLNCTYSDRGSQSFFWYRQYSGKSPELIMSIYSNGDKEDGRFTAQLNKASQYVSLLIRDSQPSDSATYLCAAVIDNDQGGILTFGTGTRLTIIPNIQNGGGGSGGGGSGGGGSGGGGSGGGGSGVTQTPRYLIKTRGQQVTLSCSPIPGHRAVSWYQQTPGQGLQFLFEYVHGEERNKGNFPGRFSGRQFSNSSSEMNISNLELGDSALYLCASSPWDSPNVQYFGPGTRLTVTEDLKNAAAGGSGGEQKLISEEDL

SEQ ID NO: 154 - protein, 18 aa, Artificial Sequence
  FLAG tag plus linkers
GGGGSDYKDDDDKGGGAS

SEQ ID NO: 155 - protein, 21 aa, Artificial Sequence
  Hinge region of IgG1
EPKSCDKTHTCPPCPAPELLG

SEQ ID NO: 156 - protein, 19 aa, Artificial Sequence
  Hinge regioin of IgG2
ERKCCVECPPCPAPPVAGP

SEQ ID NO: 157 - protein, 53 aa, Artificial Sequence
  Hinge IgG3
ELKTPLGDTTHTCPRCPEPKSCDTPPPCPRCPEPKSCDTPPPCPRCPAPELLG

SEQ ID NO: 158 - protein, 18 aa, Artificial Sequence
  Hinge IgG4
ESKYGPPCPSCPAPEFLG

SEQ ID NO: 159 - protein, 7 aa, Artificial Sequence
  Linker
GGGGSGT

SEQ ID NO: 160 - protein, 10 aa, Artificial Sequence
  Linker
GGSGGGGSGG

SEQ ID NO: 161 - protein, 15 aa, Artificial Sequence
  Linker
GGSGGGGSGGGGSGG

SEQ ID NO: 162 - protein, 12 aa, Artificial Sequence
  Linker
GGGGSGGGGSGT

SEQ ID NO: 163 - protein, 17 aa, Artificial Sequence
  Linker
GGGGSGGGGSGGGGSGT

SEQ ID NO: 164 - protein, 22 aa, Artificial Sequence
  Linker
GGGGSGGGGSGGGGSGGGGSGT

SEQ ID NO: 165 - protein, 20 aa, Artificial Sequence
  Linker
GGSGGGGSGGGGSGGGGSGG

SEQ ID NO: 166 - protein, 27 aa, Artificial Sequence
  Linker
GGGGSGGGGSGGGGSGGGGSGGGGSGT

SEQ ID NO: 167 - protein, 25 aa, Artificial Sequence
  Linker
GGSGGGGSGGGGSGGGGSGGGGSGG

SEQ ID NO: 168 - protein, 17 aa, Artificial Sequence
  Linker
GSADDAKKDAAKKDGKS

SEQ ID NO: 169 - protein, 25 aa, Artificial Sequence
  Linker
GGQGSGGTGSGGQGSGGTGSGGQGS

SEQ ID NO: 170 - protein, 20 aa, Artificial Sequence
  Linker
GGGGSGGGGSGGGGSGGGGS

SEQ ID NO: 171 - protein, 122 aa, Artificial Sequence
  VH of UCHT1(17)
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGDSDWYFDVWGQGTLVTVSS

SEQ ID NO: 172 - protein, 122 aa, Artificial Sequence
  VH of UCHT1(23)
EVQLVQSGAEVKKPGASVKVSCKASGYSFTEYTMNWVRQAPGQGLEWMGLINPQKGVSTYAQKFQDRVTLTVDKSTSTAYMELSSLRSEDTAVYYCARSGYYGDSDWYFDVWGQGTLVTVSS

SEQ ID NO: 173 - protein, 106 aa, Artificial Sequence
  VL of BMA (10)
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIK

SEQ ID NO: 174 - protein, 120 aa, Artificial Sequence
  VH of BMA (10)
EVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYDGFVYWGQGTLVTVSS

SEQ ID NO: 175 - protein, 106 aa, Artificial Sequence; identical to SEQ ID NO: 173
  VL of BMA (36)

SEQ ID NO: 176 - protein, 120 aa, Artificial Sequence
  VH of BMA (36)
EVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSS

SEQ ID NO: 177 - protein, 122 aa, Artificial Sequence
  VH of UCHT1 (17opt)
EVQLVQSGAEVKKPGASVKVSCKASGYSFTGYTMNWVRQAPGQGLEWMGLINPYKGVSTYAQKFQDRVTLTVDKSTSTAYMELSSLRSEDTAVYYCARSGYYGESDWYFDVWGQGTLVTVSS

SEQ ID NO: 178 - protein, 122 aa, Artificial Sequence
  VH of UCHT1 (21)
EVQLVQSGAEVKKPGASVKVSCKASGYSFTGYTMNWVRQAPGQGLEWMGLINPQKGVSTYAQKFQDRVTLTVDKSTSTAYMELSSLRSEDTAVYYCARSGYYGDSDWYFDVWGQGTLVTVSS

SEQ ID NO: 179 - protein, 15 aa, Artificial Sequence
  CDRa3 of Valpha A_HiAff1#29
CAAVIPNDDGGRLTF

SEQ ID NO: 180 - protein, 117 aa, Artificial Sequence
  Valpha of  A_HiAff1#29B
QKEVEQNSGPLSVPEGAIASLNCTYSDRGSQLFFWYRQYSGKSPELIMSIYQEGDKEDGRFTAQLNKASQYVSLLIRDSQPSDSATYLCAAVIPNDDGGLLTFGTGTRLTIIPNIQN

SEQ ID NO: 181 - protein, 15 aa, Artificial Sequence
  CDR3a of Valpha A_HiAff1#10
CAAVIPNDQGGYLTF

SEQ ID NO: 182 - protein, 15 aa, Artificial Sequence
  CDR3a of Valpha A_HiAff1#16
CAAVIPNNIGGALTF

(19) United States
(12) Patent Application Publication
(10) Pub. No.: US 2023/0132241 A1
(43) Pub. Date: Apr. 27, 2023

(54) ANTIGEN BINDING PROTEINS SPECIFICALLY BINDING PRAME

(71) Applicant: Immatics Biotechnologies GmbH, Tuebingen (DE)

(72) Inventors: Sebastian BUNK, Tuebingen (DE); Martin HOFMANN, Tuebingen (DE); Meike HUTT, Tuebingen (DE); Dominik MAURER, Tuebingen (DE); Gabriele PSZOLLA, Tuebingen (DE); Frank SCHWOEBEL, Tuebingen (DE); Felix UNVERDORBEN, Tuebingen (DE); Claudia WAGNER, Tuebingen (DE); Sara YOUSEF, Tuebingen (DE)

(21) Appl. No.: 17/793,237

(22) PCT Filed: Jan. 15, 2020
(86) PCT No.: PCT/EP2020/050936

§ 371 Date: Jul. 15, 2022

(51) Int. Cl. C07K 16/30; C12N 15/63; A61P 35/00

(52) U.S. Cl. CPC C07K 16/3053; C12N 15/63; A61P 35/00; C07K 2317/565; C07K 2317/567

(57) ABSTRACT

The present invention concerns antigen binding proteins directed against PRAME protein-derived antigens. The invention in particular provides antigen binding proteins which are selective and specific for the tumor expressed antigen PRAME, wherein the tumor antigen comprises or consists of SEQ ID NO: 8 and is in a complex with a major histocompatibility complex (MHC) protein. The antigen binding proteins of the invention contain, in particular, the complementary determining regions (CDRs) of novel engineered T cell receptors (TCRs) that specifically bind to said PRAME peptide. The antigen binding proteins of the invention are for use in the diagnosis, treatment and prevention of PRAME expressing cancerous diseases. Further provided are nucleic acids encoding the antigen binding proteins of the invention, vectors comprising said nucleic acids, recombinant cells expressing the antigen binding proteins and pharmaceutical compositions comprising the antigen binding proteins of the invention.

### CROSS-REFERENCE TO RELATED APPLICATIONS

This application is the National Stage entry of International Application No. PCT/EP2020/050936, filed 15 Jan. 2020, the entire contents of which are hereby incorporated by reference for all purposes.

### REFERENCE TO SEQUENCE LISTING SUBMITTED AS A COMPLIANT ASCII TEXT FILE (.txt)

Pursuant to the EFS-Web legal framework and 37 C.F.R. § 1.821-825 (see M.P.E.P. § 2442.03(a)), a Sequence Listing in the form of an ASCII-compliant text file (entitled “3000058-025000_Sequence_Listing_ST25.txt” created on Jan. 17, 2023, and 121,495 bytes in size) is submitted concurrently with the instant application, and the entire contents of the Sequence Listing are incorporated herein by reference.

### BACKGROUND

### Field

The present invention concerns antigen binding proteins directed against PRAME protein-derived antigens. The invention in particular provides antigen binding proteins which are selective and specific for the tumor expressed antigen PRAME, wherein the tumor antigen comprises or consists of SEQ ID NO: 8 and is in a complex with a major histocompatibility complex (MHC) protein. The antigen binding proteins of the invention contain, in particular, the complementary determining regions (CDRs) of novel engineered T cell receptors (TCRs) that specifically bind to said PRAME peptide. The antigen binding proteins of the invention are for use in the diagnosis, treatment and prevention of PRAME expressing cancerous diseases. Further provided are nucleic acids encoding the antigen binding proteins of the invention, vectors comprising said nucleic acids, recombinant cells expressing the antigen binding proteins and pharmaceutical compositions comprising the antigen binding proteins of the invention.

### Description of Related Art

PRAME refers to Preferentially Expressed Antigen in Melanoma and belongs to the family of germline-encoded antigens known as cancer testis antigens. Cancer testis antigens are attractive targets for immunotherapeutic intervention since they typically have limited or no expression in normal adult tissues. PRAME is expressed in a number of solid tumors as well as in leukemia and lymphomas. The peptide SLLQHLIGL (SEQ ID NO: 8) corresponds to amino acids 425-433 of the full length PRAME protein and said peptide is presented on the cell surface in complex with an MHC molecule, in particular HLA-A*02 (Kessler et al., J Exp Med. 2001 Jan. 1; 193(1):73-88).

Peptide epitopes, such as the PRAME derived peptide ‘SLLQHLIGL’, which are presented by MHC molecules, may be bound by TCRs.

While advances have been made in the development of molecular-targeting drugs for cancer therapy, there remains a need in the art to develop new anti-cancer agents that specifically target molecules highly specific to cancer cells but not normal cells.

The present invention thus refers to providing novel antigen binding proteins which are selective and specific for the tumor expressed antigen PRAME in a complex with a MHC protein and which is thus a target for, for instance, T-cell based immunotherapy.

WO2018/172533 discloses engineered TCRs and TCR based molecules that bind to the PRAME peptide comprising the amino acid sequence SLLQHLIGL of SEQ ID NO: 8 in a complex with a MHC protein complex and the use of said TCRs and TCR based molecules in the diagnosis, treatment and prevention of cancerous diseases that (over)express PRAME.

However, native T cell receptors (TCRs) specifically binding to MHC presented cancer antigens are often of lower affinity (K_{D}=1-300 μM) when compared to TCRs specifically binding to MHC presented viral antigens. Part of the explanation for this phenomenon seems to be that T cells that develop in the thymus are negatively selected (tolerance induction) on self-peptide-MHC ligands, such that T cells with too high affinity to such self-peptide-MHCs are deleted. This low affinity may be one possible explanation for tumor immune escape (Aleksic et al. 2012, Eur J Immunol. 2012 December; 42(12):3174-9). Therefore, it appears desirable to design TCR variants that bind with higher affinity to cancer antigens for use as antigen recognizing constructs in an adoptive cell therapy (ACT), or as recognition module of a soluble approach, i.e. using bispecific molecules (Hickman et al. 2016, J Biomol Screen. 2016 Sep;21(8):769-85).

However, increasing the affinity of TCRs may also increase the risk of side effects. As mentioned above, in nature high affinity TCRs directed against tumor-associated antigens, which are self-proteins, are precluded by thymic selection, to avoid recognition of self-peptides present on normal tissue through cross-reactivity. Accordingly, simply increasing the TCRs affinity for its target sequence is likely to also increase the affinity to similar non cancer-specific peptides and therefore increasing the risk of cross-reactivity and unwanted cytotoxic effects on healthy tissue. That this is not just a theoretic risk has been painfully discovered for engineered TCRs targeting MAGE-A3. In particular, previously published results have shown lethal toxicities in two patients, who were infused with T cells engineered to express a TCR targeting MAGE-A3 cross-reacting with a peptide from the muscle protein Titin, even though no cross-reactivity had been predicted in the pre-clinical studies (Linette G P et al. Blood 2013; 122:863-71, Cameron B J, et al. Sci. Transl. Med. 2013; 5: 197-103). These patients demonstrated that TCR-engineered T cells can have serious and unpredictable off-target and organ-specific toxicities.

### SUMMARY

Accordingly, there is an unmet medical need to develop and provide TCRs or antigen binding proteins specifically binding to their target with higher affinity, thus allowing to target even a tumor cell or cell lines with reduced expression of the target antigenic peptide, while a high safety profile is maintained due to a low or reduced cross-reactivity with off-target peptides (also referred to as “similar peptides”). In other words, the inventors were able to develop antigen binding proteins which exhibit high binding affinity to their target peptide while maintaining high tumor selectivity.

Furthermore, as shown by the inventors, the TCR molecule R16P1C10 shows a relatively low solubility as a single chain construct or in the bispecific format referred to as TCER (in the following “TCER®”), which comprises a part that specifically binds to a surface molecule on a T cell and that specifically binds to a MHC-peptide complex.

Therefore, despite these advancements in TCR technology, there remains a need for additional cancer therapeutics, particularly those that efficiently target and kill cancer cells. In particular there is the need to develop new antigen binding proteins which are i) selective and specific for the tumor expressed PRAME derived peptide in a complex with an MHC protein having the desired biological activity, ii) have good metabolic, pharmacokinetic or safety profiles, and iii) that can be manufactured in large scale compatible with industrial practice.

Accordingly, in context of the present invention, the inventors engineered several antigen binding proteins comprising the CDR variants derived from said parental TCR R16P1C10. These antigen binding proteins have an increased binding affinity for the peptide-MHC complex and an increased stability, and/or an increased solubility, making them more suitable for a medical use.

Furthermore, the antigen binding proteins of the invention, in particular F_{c}-containing bispecific TCR/mAb diabodies (TCER® molecules or simply TCER®) comprising the variable alpha and beta domains of the novel TCR variants, exert high cytotoxicity against tumor cells wherein the half maximal effective concentration (EC₅₀) is between 10 pM and 150 pM.

For example, for antigen binding proteins of the invention, such as TCER® molecules, the half maximal effective concentration (EC₅₀) against UACC257, Hs695T cells and U2OS is in the range of 1 to 600 pM, 1 to 300 pM, 1 pM to 100 pM, more particularly, between 1 pM and 20 pM. Furthermore, the EC₅₀ of the antigen binding proteins of the present invention is 100-fold, preferably more than 1000-fold higher in healthy tissue cell lines such as primary cell lines versus tumor cell lines, demonstrating its increased safety.

Furthermore, the inventors demonstrated significant tumor growth inhibition in a therapeutic in vivo mouse model for antigen binding proteins of the invention (TCER® molecules), at low doses.

The present invention thus, refers to antigen binding proteins selective and specific for the tumor expressed antigen PRAME in a complex with an MHC protein comprising mutations leading to an increased binding affinity and an increased stability, such as reduced aggregation during expression and/or purification. Furthermore, said antigen binding proteins have a high cytotoxicity, for example against UACC257 cells, HS695T cells and U2OS cells.

In summary, the surprising findings of the inventors provide inter alia the following advantages over the art: (i) reduction of cross-reactivity of TCRs or antigen binding proteins with similar peptides on healthy tissues and maintaining high tumor selectivity (ii) increased safety profile of TCRs or antigen binding proteins; (iii) TCRs or antigen binding proteins exerting reduced off target and off-tumor cytotoxicity; and (iv) the provision of improved specific, selective and safe TCRs or antigen binding proteins.

### BRIEF DESCRIPTION OF THE DRAWINGS

FIGS. 1-13 depict embodiments as described herein.

FIG. 1: Results of stress stability studies of bispecific molecules containing the variable domains of TCR variants CDR6, LoAff3 or HiAff1, respectively. Molecular integrity and aggregate formation were analyzed by SEC-HPLC after incubation at 40° C. for 2 weeks (upper traces in gray) and compared to non-stressed molecules stored at 2-8° C. (lower traces in black). Lower panel of graphs represent the graphs from the upper panel at increased magnification.

FIG. 2: Results of stress stability studies of bispecific molecules containing the variable domains of TCR variants CDR6, LoAff3 or HiAff1, respectively. Recovery of monomeric bispecific molecules was calculated according to HPLC-SEC analyses.

FIG. 3: Binding kinetics of bispecific molecules comprising different R16P1C10 variants. FAB2G sensors were used for the scTCR-Fab format (20 μg/ml loaded for 120 s), AHC sensors for the diabody-F_{c} formats (10 μg/ml loaded for 120 s for improved variant; 5 μg/ml loaded for 120 s for stabilized variant, LoAff3, CDR6, HiAff1). Analyzed concentrations of HLA-A*02/PRAME-004 are represented in nM. Graphs show curves of measured data and calculated fits.

FIG. 4: Lysis of PRAME-positive tumor cell lines induced by bispecific molecules containing CDR6, HiAff 1 or LoAff3 TCR variants, respectively, in presence of CD8+ T cells derived from two healthy donors (HBC-887 and HBC-889). Lysis was determined after 48 hours of coincubation by quantification of released LDH. CDR6 is shown as black circle, HiAff1 as light gray square, LoAff3 as dark gray triangle, and the control group without bsTCR as open inverted triangle, respectively.

FIG. 5: Lysis of PRAME-negative tumor cell lines induced by bispecific molecules containing CDR6, HiAff 1 or LoAff3 TCR variants, respectively, in presence of CD8+ Tcells derived from two healthy donors (HBC-887 and HBC-889). Lysis was determined after 48 hours of coincubation by quantification of released LDH. CDR6 is shown as black circle, HiAff1 as light gray square, LoAff3 as dark gray triangle, and the control group without bsTCR as open inverted triangle, respectively.

FIG. 6: In vivo efficacy. NOG mice bearing HS695T tumors of approximately 50 mm³ were transplanted with human PBMCs and treated with PBS (group 1), 0.5 mg/kg body HiAff1/antiCD3 diabody-Fc (group 2) or 0.5 mg/kg antiHlV/antiCD3 diabody-Fc (group) i.v. twice a week. Tumor volumes were measured with a caliper and calculated by length x width² /2.

FIG. 7: Binding motif analysis. KD values of HiAff1 and LoAff3 variants (as diabody-F_{c}) for binding to PRAME-004 and peptides comprising sequential alanine substitutions (_A1 to _A9) were measured by biolayer interferometry. Respective ratios K_{D}(PRAME-004)/K_{D}(Ala substitution variant) were calculated.

FIG. 8: PRAME-004 and off-target peptide recognition scTCR variants derived from a CDRa3 library originating from scTCR CDR6 were displayed on the yeast surface and analyzed for binding towards PRAME-004 and the off-target peptides IFT17-003, IFIT1-001, FADS2-001 and CTBP1-001 in the context of HLA-A*02, respectively. All HLA-A*02/peptide complexes were added to the scTCR displaying yeast at a concentration of 100 nM and the percentage of positive yeast cells was determined via flow cytometry analysis.

FIG. 9: PRAME-004 and off-target peptide recognition

FIG. 10: TCER®-mediated in vivo efficacy in human PBMC-engrafted, immunodeficient NOG mice. TCER®-mediated in vivo efficacy in human PBMC-engrafted, immunodeficient NOG mice against tumor xenografts of NCI-H1755 cells, an HLA-A*02-positive tumor cell line with detectable presentation of PRAME-004. Mice were treated twice weekly with a PRAME-004-targeting TCER (HiAff¹/ₐntiCD3 diabody-Fc, 0.5 mg/(kg body weight x dose), group 2) or the vehicle (group 1).

Complete remission is shown for the PRAME-004-targeting TCER®. Tumor volumes were measured with a caliper and calculated by length×width²/2. Each symbol connected by a line represents an individual animal and treatment days are indicated with an arrow. Three animals died in group 1 and group 2 at Day 22.

FIG. 11 In vitro safety assessment with healthy tissue cells. In vitro safety data for TCER® molecule HiAff1 was assessed in LDH-killing experiments with different human HLA-A*02-positive normal tissue primary cells from different organs. The different cells were co-cultured with PBMC effector cells from healthy HLA-A*02+ donors at a ratio of 1:10 and increasing TCER® concentrations. Cells were co-incubated in a 50% mixture of primary tissue cell-specific medium and T cell medium. Each plot shows LDH data of the tumor control cell line Hs695T (black circles) and the primary normal cells (grey triangles) in the same medium combination. The no TCER control on the tumor cell line is depicted as black crossed circle and on the primary normal cell with as grey filled triangle.

FIG. 12 In vitro cytokine release assessment of TCER® molecules. Analysis of cytokine secretion induced by TCER® molecules in whole blood allows for selection of TCER® molecules with low or absent cytokine induction. Cytokine release was assessed in whole blood of three HLA-A*02-positive donors after 48 h incubation in the presence of indicated concentrations of HiAffi TCER® or anti-CD3 control antibody and human umbilical vein endothelial cells (HUVEC). Cytokine concentrations in the supernatant were determined by multiplexed analysis. Exemplary data of IFNγ and IL-6 release are shown.

FIG. 13: HLA-A*02-dependent presentation of PRAME derived target peptide in cancer and in normal tissues. Abbreviations are as follows: adipose: adipose tissue; adrenal gl: adrenal gland; bladder: urinary bladder; bloodvess: blood vessel; esoph: esophagus; gall bl: gallbladder; intest. la: large intestine; intest. sm: small intestine; nerve cent: central nerve; nerve periph: peripheral nerve; parathyr: parathyroid gland; perit: peritoneum; pituit: pituitary; skel. mus: skeletal muscle; AML: acute myeloid leukemia; BRCA: breast cancer; CCC: cholangiocellular carcinoma; CLL: chronic lymphocytic leukemia; CRC: colorectal cancer; GBC: gallbladder cancer; GBM: glioblastoma; GC: gastric cancer; GEJC: Gastro-esophageal junction cancer; HCC: hepatocellular carcinoma; HNSCC: head and neck squamous cell carcinoma; MEL: melanoma; NHL: non-Hodgkin lymphoma; NSCLCadeno: non-small cell lung cancer adenocarcinoma; NSCLCother: NSCLC samples that could not unambiguously be assigned to NSCLC adeno or NSCLCsquam; NSCLCsquam: squamous cell non-small cell lung cancer; OC: ovarian cancer; OSCAR: esophageal cancer; PACA: pancreatic cancer; PRCA: prostate cancer; RCC: renal cell carcinoma; SCLC: small cell lung cancer; UBC: urinary bladder carcinoma; UEC: uterine and endometrial cancer.

### DETAILED DESCRIPTION OF A PREFERRED EMBODIMENT

### Definitions

The term “Antigen” or “Target Antigen” as used herein refers to a molecule or a portion of a molecule or complex that is capable of being bound by one antigen binding site, wherein said one antigen binding site is, for example, present in a conventional antibody, a conventional TCR and/or other antigen binding proteins of the present invention. The antigen in context of the present invention is in particular the PRAME peptide comprising or consisting of the amino acid sequence SLLQHLIGL of SEQ ID NO: 8, more particularly the PRAME peptide comprising the amino acid sequence of SEQ ID NO: 8, more particularly PRAME peptide comprising the amino acid sequence SLLQHLIGL of SEQ ID NO: 8 in a complex with a MHC protein, such as a HLA protein, for instance HLA-A*02.

The term “Antigen Binding Protein” herein refers to polypeptides or binding proteins that are able to bind to one antigen. In a preferred embodiment, said antigen-binding proteins are able of binding to two different antigens simultaneously, as it is known from, for example bispecific antibodies. However, unlike conventional antibodies as defined herein the antigen binding proteins of the present invention comprise at least 6 CDRs from a TCR. In a particular embodiment, the antigen binding proteins of the present invention, unlike conventional antibodies comprise at least one variable alpha domain and at least one variable beta domain from a TCR.

A “Domain” may be any region of a protein, generally defined on the basis of sequence homologies and often related to a specific structural or functional entity. Examples of such domains are the variable light chain domain of the antibody light chain, the variable heavy chain domain of the antibody heavy chain and the constant domains (C_{H1}, C_{H2} and C_{H3} domain) of the antibody heavy chain, the variable domain of the a-chain of a TCR or the variable domain of the β-chain of a TCR.

The term “Epitope” as used herein comprises the terms “structural epitope” and “functional epitope”. The “Structural Epitope” are those amino acids of the antigen, e.g. peptide-MHC complex, that are covered by the antigen binding protein when bound to the antigen. Typically, all amino acids of the antigen are considered covered that are within 5 Å of any atom of an amino acid of the antigen binding protein. The structural epitope of an antigen may be determined by art known methods including X-ray crystallography or NMR analysis. The structural epitope of an antibody typically comprises 20 to 30 amino acids. The structural epitope of a TCR typically comprises 20 to 30 amino acids. The “Functional Epitope” is a subset of those amino acids forming the structural epitope and comprises the amino acids of the antigen that are critical for formation of the interface with the antigen binding protein of the invention, either by directly forming non-covalent interactions such as H-bonds, salt bridges, aromatic stacking or hydrophobic interactions or by indirectly stabilizing the binding conformation of the antigen and is, for instance, determined by mutational scanning. Typically, the functional epitope of an antigen bound by an antibody comprises between 4 and 6 amino acids. Typically, the functional epitope of a peptide-MHC complex comprises between 2 to 6 amino acids of the peptide and 2 to 7 amino acids of the MHC molecule. Since MHC I presented peptides typically have a length between 8 to 10 amino acids only a subset of amino acids of each given peptide is part of the functional epitope of a peptide-MHC complex. In context of the present invention, the epitope, in particular the functional epitope bound by the antigen binding proteins of the present invention comprises or consists of the amino acids of the antigen that are required for formation of the binding interface, therefore the functional epitope comprises at least 3, preferably at least 4 amino acids of the PRAME-004 antigenic peptide of SEQ ID NO: 8.

“PRAME” or “Preferentially Expressed Antigen In Melanoma” was first identified as an antigen that is over expressed in melanoma (Ikeda et al Immunity. 1997 February; 6(2): 199-208); it is also known as CT130, MAPE, 01P-4 and has the Uniprot accession number P78395 (as available on January 11, 2019). The protein functions as a repressor of retinoic acid receptor signaling (Epping et al., Cell. 2005 Sep. 23; 122(6):835-47). PRAME belongs to the family of germline-encoded antigens known as cancer testis antigens. Cancer testis antigens are attractive targets for immunotherapeutic intervention since they typically have limited or no expression in normal adult tissues. PRAME is expressed in a number of solid tumors as well as in leukemia and lymphomas (Doolan et al., Breast Cancer Res Treat. 2008 May; 109(2):359-65; Epping et al., Cancer Res. 2006 Nov. 15; 66(22): 10639-42; Ercolak et al., Breast Cancer Res Treat. 2008 May; 109(2):359-65; Matsushita et al., Leuk Lymphoma. 2003 March; 44(3):439-44; Mitsuhashi et al., Int. J Hematol. 2014; 100(1):88-95; Proto-Sequeire et al., Leuk Res. 2006 November; 30(11): 1333-9; Szczepanski et al., Oral Oncol. 2013 February; 49(2): 144-51; Van Baren et al., Br J Haematol. 1998 September; 102(5): 1376-9). PRAME targeting therapies of the inventions may be particularly suitable for treatment cancers including, but not limited to, lung cancer, such as non-small cell lung cancer, small cell lung cancer, liver cancer, head and neck cancer, skin cancer, renal cell cancer, brain cancer, gastric cancer, colorectal cancer, hepatocellular cancer, pancreatic cancer, prostate cancer, leukemia, breast cancer, Merkel cell carcinoma, melanoma, ovarian cancer, urinary bladder cancer, uterine cancer, gallbladder and bile duct cancer, and esophageal cancer.

The “PRAME Derived Peptide” comprises or consist of the amino acid sequence SLLQHLIGL (SEQ ID NO: 8) which corresponds to amino acids 425-433 of the full length PRAME protein of the amino acid sequence of SEQ ID NO: 7 as accessible under the Uniprot accession number P78395 (as available on January 11, 2019).). The PRAME derived peptide which comprises or consist of the amino acid sequence SLLQHLIGL (SEQ ID NO: 8) is also herein referred to as PRAME-004. The PRAME-004 peptide is a peptide epitope derived from a tumor-associated or tumor-specific protein and is presented on the cell surface by molecules of the major histocompatibility complex (MHC).More particularly, the PRAME-004 derived peptide is presented on the cell surface in complex with HLA-A*02. Med. 2001 Jan. 1; 193(1):73-88). In context of the invention, the “PRAME derived peptide” or “PRAME-004” are used interchangeably and thus refer to PRAME derived peptide comprising or consisting of the amino acid sequence SLLQHLIGL (SEQ ID NO: 8).

The “Major Histocompatibility Complex” (MHC) is a set of cell surface proteins essential for the acquired immune system to recognize foreign molecules in vertebrates, which in turn determines histocompatibility. The main function of MHC molecules is to bind to antigens derived from pathogens and display them on the cell surface for recognition by the appropriate T-cells. The human MHC is also called the HLA (human leukocyte antigen) complex (often just the HLA).

The MHC gene family is divided into three subgroups: class I, class II, and class III. Complexes of peptide and MHC class I are recognized by CD8-positive T-cells bearing the appropriate T-cell receptor (TCR), whereas complexes of peptide and MHC class II molecules are recognized by CD4-positive-helper-T-cells bearing the appropriate TCR. Since both types of response, CD8 and CD4 dependent, contribute jointly and synergistically to the anti-tumor effect, the identification and characterization of tumor-associated antigens and corresponding T cell receptors is important in the development of cancer immunotherapies such as vaccines and cell therapies. The HLA-A gene is located on the short arm of chromosome 6 and encodes the larger, α-chain, constituent of HLA-A. Variation of HLA-A a-chain is key to HLA function. This variation promotes genetic diversity in the population. Since each HLA has a different affinity for peptides of certain structures, greater variety of HLAs means greater variety of antigens to be ‘presented’ on the cell surface. Each individual can express up to two types of HLA-A, one from each of their parents. Some individuals will inherit the same HLA-A from both parents, decreasing their individual HLA diversity; however, the majority of individuals will receive two different copies of HLA-A. This same pattern follows for all HLA groups. In other words, every single person can only express either one or two of the 2432 known HLA-A alleles.

“HLA-A*02” signifies a specific HLA allele, wherein the letter A signifies the allele and the prefix “*02 prefix” indicates the A2 serotype.

In the MHC class I dependent immune reaction, peptides not only have to be able to bind to certain MHC class I molecules expressed by tumor cells, they subsequently also have to be recognized by T-cells bearing specific T-cell receptors (TCR).

A “TCR” is a heterodimeric cell surface protein of the immunoglobulin super-family, which is associated with invariant proteins of the CD3 complex involved in mediating signal transduction. TCRs exist in αβ and γδ forms, which are structurally similar but have quite distinct anatomical locations and probably functions. The extracellular portion of native heterodimeric αβ TCR and γδ TCR each contain two polypeptides, each of which has a membrane-proximal constant domain, and a membrane-distal variable domain. Each of the constant and variable domains include an intra-chain disulfide bond. The variable domains contain the highly polymorphic loops analogous to the complementarity determining regions (CDRs) of antibodies. The use of TCR gene therapy overcomes a number of current hurdles. It allows equipping subjects' (patients') own T cells with desired specificities and generation of sufficient numbers of T cells in a short period of time, avoiding their exhaustion. For example, the TCR will be, transduced into potent T cells (e.g. central memory T cells or T cells with stem cell characteristics), which may ensure better persistence, preservation and function upon transfer. TCR-engineered T cells will be infused into cancer patients rendered lymphopenic by chemotherapy or irradiation, allowing efficient engraftment but inhibiting immune suppression.

The term “TCR” herein denotes TCRs and fragments thereof, as well as single chain TCRs and fragments thereof, in particular, variable alpha and beta domains of single domain TCRs, and chimeric, humanized, bispecific or multispecific TCRs.

“Fragments of a TCR” comprise a portion of a native TCR or conventional TCR, in particular the antigen binding region or variable region of the native TCR. Examples of TCR fragments include fragments of the α, β, δ, γ chain, such as V_{α}-Cₐ or V_{β}-C_{β} or portions thereof, such fragments might also further comprise the corresponding hinge region or single variable domains, such as Vα, Vβ, Vδ, Vγ, single chain VαVβ fragments or bispecific and multispecific TCRs formed from TCR fragments. Fragments of a TCR comprise a portion of an intact TCR, in particular the antigen binding region or variable region of the intact TCR, and thus selectively and specifically bind to their target peptide compared to the naturally occurring full-length TCR.

“Single Chain TCR (scTCR)” herein denotes a protein wherein the variable domains of the TCR, such as the V_{α} and V_{β} or V_{δ} and V_{γ} are located on one polypeptide. Typically, the variable domains in scTCR are separated by a linker, wherein said linker typically comprises 5 to 20, such as 5 to 15 amino acids.

“Native” as used for example in the wording “native TCR” refers to a wildtype TCR.

In the adoptive cell transfer (ACT) approach, the transgene T cells produced may express a wildtype TCR having wildtype alpha and beta chains, and the transgenic TCR with the alpha and beta chains specific for the respective recombinant antigen-MHC complex. Both the wildtype alpha and beta chains and the transgenic alpha and beta chains are usually still capable of cross-pairing with each other. This undesired pairing is called “TCR mispairing”, and is a recognized problem in the field of TCR (gene) therapy. Mispairing and incorrect pairing between a transgenic TCR α or β chain and an endogenous TCR β or α chain, respectively, may result in a reduced surface expression of the transgenic TCRαβ heterodimer, which in turn reduces the functional avidity of the modified T cells. Furthermore, T cells expressing mispaired TCRs and expanded under high IL-2 conditions were demonstrated to induce graft-versus-host disease (GvHD) in a preclinical model.

Some strategies for the optimization of transgenic TCR α and β pairing to avoid mispairing may include the following: 1. Murinized TCRs: In this approach, human TCRα and β constant chains are replaced by the corresponding murine domains. Although human and murine TCR-C domains show a high degree of homology, small differences affect the stability of TCR/CD3 interactions and hence TCR surface expression levels. 2. Cysteine-modified TCRs: This approach introduces cysteine amino acids at a structurally favorable position, and hence allows the formation of an additional disulfide bridge and promotes correct pairing between the two TCR chains. Site-directed mutations of e.g. T48C in the TCR alpha constant chain and S57C in the TCR beta constant chain resulted in a TCR heterodimer linked by two interchain bonds (i.e., an introduced disulfide bridge plus an endogenous transmembrane disulfide bridge (position No. 95 in the alpha constant domain and position No. 131 in the beta constant domain). 3. Domain swapping: Constant domains are swapped between α and β chains of a tumor-specific T cell receptor, creating a domain-swapped (ds) TCR. When correctly paired, these dsTCR chains retain all domains necessary to recruit CD3 proteins, to express on the T cell surface, and to mediate functional T cell responses upon engaging a target antigen. By contrast, mispaired TCRs containing one dsTCR chain and one wild-type TCR chain lack key domains necessary for CD3 recruitment, export, and signaling, and thus are unable to mediate deleterious autoimmunity. 4. Exclusive TCR heterodimers: In this approach, sterical and electrostatic forces are exploited to facilitate correct pairing between TCR alpha and beta transgenes and at the same time inhibit pairing between exogenous and endogenous TCR alpha and beta chains. One example uses site-directed mutations to introduce an S85R into the alpha constant domain and R88G in the beta constant domain, in order to obtain the required changes in electrostatic charges, and hence generate a reciprocal ‘knob-into-hole’ configuration, which allegedly minimally distorts secondary and tertiary structures. 5. The use of chimericTCR-CD3ζ chain having each TCR chain fused to a CD3ζ molecule. 6. The use of single-chain TCRs wherein the Vα of a defined TCR is fused to the beta chain using a flexible peptide linker. 7. The use of shRNA sequences or zinc finger nucleases to knock down the expression of the endogenous TCR. Another approach, termed “velcro”, that is able to pair the two TCR chains with one another due to favorable electrostatic interactions in the heterodimeric state. That is, the two peptides are predominantly unfolded in isolated form but associate preferentially to form a stable parallel, coiled coil heterodimer when mixed. This approach may be applied to produce soluble TCR, in which heterodimeric complex was favored by fusing the peptides to truncated alpha and beta chains respectively.

Native alpha-beta heterodimeric TCRs have an alpha chain and a beta chain. Each alpha chain comprises variable, joining and constant regions, and the beta chain also usually contains a short diversity region between the variable and joining regions, but this diversity region is often considered as part of the joining region. The constant, or C, regions of TCR alpha and beta chains are referred to as TRAC and TRBC respectively (Lefranc, (2001), Curr Protoc Immunol Appendix 1: Appendix 10). Each variable region, herein referred to as alpha variable domain and beta variable domain, comprises three Complementarity Determining Regions (CDRs) embedded in a framework sequence, one being the hypervariable region named CDR3. The alpha variable domain CDRs are herein referred to as CDRa1, CDRa2, CDRa3, and the beta variable domain CDRs are herein referred to as CDRb1, CDRb2 and CDRb3. There are several types of alpha chain variable (Valpha) regions and several types of beta chain variable (Vbeta) regions distinguished by their framework, CDR1 and CDR2 sequences, and by a partly defined CDR3 sequence. The Valpha types are referred to in IMGT nomenclature by a unique TRAV number, Vbeta types are referred in IMGT nomenclature to by a unique TRBV number (Folch and Lefranc, (2000), Exp Clin Immunogenet 17(1): 42-54; Scaviner and Lefranc, (2000), Exp Clin Immunogenet 17(2): 83-96; LeFranc and LeFranc, (2001), “T cell Receptor Factsbook”, Academic Press). For more information on immunoglobulin antibody and TCR genes see the international ImMunoGeneTics information system®, Lefranc M-P et al., (Nucleic Acids Res. 2015 January; 43(Database issue):D413-22; and imgt.org/). A native or conventional TCR antigen-binding site, therefore, includes, usually, six CDRs, comprising the CDR set from each of an alpha and a beta chain variable region, wherein CDR1 and CDR3 sequences are relevant to the recognition and binding of the peptide antigen that is bound to the HLA protein and the CDR2 sequences are relevant to the recognition and binding of the HLA protein.

Analogous to antibodies, “TCR Framework Regions” (FRs) refer to amino acid sequences interposed between CDRs, i.e. to those portions of TCR alpha and beta chain variable regions that are to some extent conserved among different TCRs. The alpha and beta chains of a TCR each have four FRs, herein designated FR1-a, FR2-a, FR3-a, FR4-a, and FR1-b, FR2-b, FR3-b, FR4-b, respectively. Accordingly, the alpha chain variable domain may thus be designated as (FR1-a)-(CDRa1)-(FR2-1)-(CDRa2)-(FR3-a)-(CDRa3)-(FR4-a) and the beta chain variable domain may thus be designated as (FR1-b)-(CDRb1)-(FR2-b)-(CDRb2)-(FR3-b)-(CDRb3)-(FR4-b).

In the context of the invention, CDR/FR definition in an α or β chain or a γ or δ chain is to be determined based on IMGT definition (Lefranc et al., Dev. Comp. Immunol., 2003, 27(1):55-77; imgt.org). Accordingly, CDR/FR amino acid positions when related to TCR or TCR derived domains are indicated according to said IMGT definition. Preferably, the IMGT position of the CDR/FR amino acid positions of the first variable domain is given in analogy to the IMGT numbering of TRAV12-2and/or the IMGT position of the CDR/FR amino acid positions of the second variable domain is given in analogy to the IMGT numbering of TRBV5-1.

“Affinity” is defined, in theory, by the equilibrium binding between the antigen binding protein and the antigen, in context of the present invention by the equilibrium binding between the antigen binding protein and the antigen, namely the PRAME-004 peptide according to SEQ ID NO: 8 in a complex with a MHC protein. Affinity may be expressed for example in half-maximal effective concentration (EC₅₀) or the equilibrium dissociation constant (K_{D}).

“K_{D}” is the equilibrium dissociation constant, a ratio of k_{off}/kₒₙ, between the antigen binding protein and its antigen. K_{D} and affinity are inversely related. The K_{D} value relates to the concentration of the antigen binding protein and the lower the K_{D} value, the higher the affinity of the antigen binding protein. Affinity, i.e. the K_{D} value can be experimentally assessed by a variety of known methods, such as measuring association and dissociation rates with surface Plasmon resonance or biolayer interferometry, as described herein below in the section ‘Antigen binding proteins’.

In an “Antibody”, also called “Immunoglobulin”, two heavy chains are linked to each other by disulfide bonds and each heavy chain is linked to a light chain by a disulfide bond. There are two types of light chain, lambda (λ) and kappa (κ). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (V_{L}) and a constant domain (C_{L}). The heavy chain includes four domains, a variable domain (V_{H}) and three constant domains (C_{H1}, C_{H2} and C_{H3}, collectively referred to as C_{H}). The variable regions of both light (V_{L}) and heavy (V_{H}) chains determine binding recognition and specificity to the antigen. The constant region domains of the light (C_{L}) and heavy (C_{H}) chains confer important biological properties such as antibody chain association, secretion, trans-placental mobility, complement binding, and binding to F_{c} receptors (F_{c}R). The Fv fragment is the N-terminal part of the Fab fragment of an immunoglobulin and consists of the variable portions of one light chain and one heavy chain. The specificity of the antibody resides in the structural complementarity between the antibody combining site (synonym to antibody binding site) and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. CDRs refer to amino acid sequences that together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region. In context of the present invention

In context of the invention, the antibody or immunoglobulin is an IgM, IgD, IgG, IgA and IgE.

“Antibody Framework Regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively. Accordingly, the light chain variable domain may thus be designated as (FR1-L)-(CDR1-L)-(FR2-L)-(CDR2-L)-(FR3-L)-(CDR3-L)-(FR4-L) and the heavy chain variable domain may thus be designated as (FR1-H)-(CDR1-H)-(FR2-H)-(CDR2-H)-(FR3-H)-(CDR3-H)-(FR4-H).

In the context of the invention, CDR/FR definition in an immunoglobulin light or heavy chain, except of the F_{c} domain, is to be determined based on IMGT definition (Lefranc et al., Dev. Comp. Immunol., 2003, 27(1):55-77; imgt.org). Accordingly, amino acid sequences of the CDR1, CDR2 and CDR3 of a given variable chain and the amino acid sequences of FR1, FR2, FR3, FR4 and FR5 are indicated according to said IMGT definition.

As it will be understood by the person skilled in the art, the structures of antibodies, in particular the structure of the variable heavy and light chains of antibodies is analogous to the structures of the TCR alpha and beta variable domain structure facilitating the grafting of the CDRs as defined in context of the present invention into conventional antibodies, bispecific antibodies, or multispecific antibodies.

Knowing the amino acid sequence of the CDRs of an antibody, a TCR or an antigen binding protein of the invention, one skilled in the art can easily determine the framework regions, such as the TCR framework regions or antibody framework regions. However, in cases where the CDRs are not indicated, the skilled in the art can first determine the CDR amino acid sequences based on the IMGT definition for TCRs or the IMGT definition for antibodies and then determine the amino acid sequences of the framework regions.

For example, knowing the amino acid sequence of the CDRs one skilled in the art can easily determine the framework regions FR1-L, FR2-L, FR3-L, FR4-L and/or FR1-H, FR2-H, FR3-H, FR4-H.

As used herein, a “Human Framework Region” is a framework region that is substantially identical (about 85%, or more, in particular 90%, 95%, 97%, 99% or 100%) to the framework region of a naturally occurring human antibody.

As used herein, the term “Antibody” denotes antibodies and fragments thereof, such as conventional antibodies and fragments thereof, as well as single domain antibodies and fragments thereof, in particular a variable heavy chain of a single domain antibodies, and chimeric, humanized, bispecific or multispecific antibodies.

A “Conventional Antibody” as herein referred to be an antibody that has the same type of domains and domain arrangements as an antibody isolated from nature and comprises antibody derived CDRs and framework regions. In analogy, a “Conventional TCR” as herein referred to is a TCR that comprises the same type of domains and domain arrangement as a native TCR and TCR derived CDRs and Framework regions.

The term “Humanized Antibody” refers to an antibody which is completely or partially of non-human origin and which has been modified by replacing certain amino acids, in particular in the framework regions of the heavy and light chains, in order to avoid or minimize an immune response in humans. The constant domains of a humanized antibody are mainly human C_{H} and C_{L} domains.

Numerous methods for humanization of an antibody sequence are known in the art; see e.g. the review by Almagro & Fransson (2008) Front Biosci. 13: 1619-1633. One commonly used method is CDR grafting, or antibody reshaping, which involves grafting of the CDR sequences of a donor antibody, generally a mouse antibody, into the framework scaffold of a human antibody of different specificity. Since CDR grafting may reduce the binding specificity and affinity, and thus the biological activity, of a CDR grafted non-human antibody, back mutations may be introduced at selected positions of the CDR grafted antibody in order to retain the binding specificity and affinity of the parent antibody. Identification of positions for possible back mutations can be performed using information available in the literature and in antibody databases. Amino acid residues that are candidates for back mutations are typically those that are located at the surface of an antibody molecule, while residues that are buried or that have a low degree of surface exposure will not normally be altered. An alternative humanization technique to CDR grafting and back mutation is resurfacing, in which non-surface exposed residues of non-human origin are retained, while surface residues are altered to human residues. Another alternative technique is known as “guided selection” (Jespers et al., (1994) Biotechnology 12, 899) and can be used to derive from, for example, a murine or rat antibody, a fully human antibody conserving the epitope and binding characteristics of the parental antibody. A further method of humanization is the so-called 4D humanization as described in US20110027266 A1 (WO2009032661A1).

For chimeric antibodies, humanization typically involves modification of the framework regions of the variable region sequences.

Amino acid residues that are part of a CDR will typically not be altered in connection with humanization, although in certain cases it may be desirable to alter individual CDR amino acid residues, for example to remove a glycosylation site, a deamidation site or an undesired cysteine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, in particular by way of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in a CDR sequence, it may therefore be desirable to remove the site, typically by conservative substitution to remove one of the implicated residues. Substitution in a CDR sequence to remove one of the implicated residues is also intended to be encompassed by the present invention.

“Fragments” of antibodies, for example of conventional antibodies, comprise a portion of an intact antibody, in particular the antigen binding region or variable region of the intact antibody. Examples of antibody fragments include Fv, Fab, F(ab′)2, Fab′, dsFv, (dsFv)2, scFv, sc(Fv)2, diabodies, bispecific and multispecific antibodies formed from antibody fragments. A fragment of a conventional antibody may also be a single domain antibody, such as a heavy chain antibody or VHH.

The term “Fab” denotes an antibody fragment having a molecular weight of about 50,000 Dalton and antigen binding activity, in which about a half of the N-terminal side of H chain and the entire L chain, among fragments obtained by treating IgG with a protease, e.g. papain, are bound together through a disulfide bond.

The term “Bispecific” for example in a “Bispecific Antibody” or “BsAb” usually denotes an antibody, which combines the antigen-binding sites of two antibodies within a single molecule. Thus, BsAbs are able to bind two different antigens simultaneously.

In particular, the term “Bispecific” in the context of the present invention refers to antigen binding proteins with at least two valences and binding specificities for two different antigens and thus, comprises two antigen binding sites, in other words, a bispecific antigen binding protein refers to a binding protein that specifically binds two different targets.

The term “Valence” refers to the number of binding sites of an antigen binding protein, e.g. a bivalent antigen binding protein relates to an antigen binding protein that has two binding sites. It should be noted, that, the term valence refers to the number of binding sites, wherein those binding sites may bind to the same or different targets, i.e. a bivalent antigen binding protein may be monospecific, i.e. binding one target, or bispecific, i.e. binding two different targets. Targets may be antigens, target peptides, off-targets, i.e. similar peptides or the like. In context of the present invention it is preferred that at least one antigen-binding site is derived from a TCR, more particularly, that at least one antigen binding site comprises the TCR derived CDRs as defined in context of the present invention. Accordingly, “Bispecific” in context of the present invention refers to an antigen binding protein which combines at least one antigen-binding site comprising TCR CDRs and one further antigen binding site, wherein said further antigen binding site, is typically derived from an antibody and thus, typically comprises antibody CDRs.

The term “Bispecific Antibody” or “BsAb” accordingly denotes, in context of the present invention, a bispecific protein in an antibody format, which combines one antigen-binding site comprising TCR CDRs and one antigen binding site comprising antibody CDRs within a single molecule. Thus, BsAbs are able to bind two different antigens simultaneously. Genetic engineering has been used with increasing frequency to design, modify, and produce antibodies or antibody derivatives with a desired set of binding properties and effector functions as described for instance in EP 2 050 764 A1.

The term “Format” herein refers to the number and type of domains that are present in an antigen binding protein of the invention and the spatial organization thereof. For example, in context of the antigen binding protein of the invention, the antigen binding protein may comprise a first polypeptide with Valpha-L-VL(CD3)-FC and a second polypeptide with VH(CD3)-L-Vbeta-F_{c} and is thus in a different format than an antigen binding protein without the F_{c} domains. L is a linker linking the Valpha and VL domain and the VH and Vbeta domain, respectively.

Many different formats, such as bispecific formats, are described in the art. In context of antibodies, such formats typically include non-limiting examples, such as, diabodies, Cross-Over-Dual-Variable-Domain (CODV) and/or dual variable domain (DVD) proteins. An overview of these different bispecific antibodies and ways of producing are disclosed in, for example, Brinkmann U. and Kontermann E. E. MAbs. 2017 February-March; 9(2): 182-212. Particularly, the DVD format is, for example, disclosed in the following scientific articles (Wu C et al. Nat Biotechnol 2007; 25:1290-7; PMID:17934452; Wu C. et al. MAbs 2009; 1:339-47; Lacy S E et al. MAbs 2015; 7:605-19; PMID:25764208; Craig R B et al. PLoS One 2012; 7:e46778; PMI D:23056448; Piccione E C et al. MAbs 2015). The CODV is for example disclosed in Onuoha S C et al. Arthritis Rheumatol. 2015 October; 67(10):2661-72 or for example in WO2012/135345, WO2016/116626. Bispecific diabodies are for example described in Holliger P et al. Protein Eng 1996; 9:299-305; PMID:8736497; Atwell J L et al. Mol Immunol 1996; 33:1301-12; PMID:9171890; Kontermann R E, Nat Biotechnol 1997; 15:629-31; PMID:9219263; Kontermann R E et al. Immunotechnology 1997; 3:137-44; PMID:9237098; Cochlovius B et al. Cancer Res 2000; 60:4336-41; PMID:10969772; and DeNardo D G et al. Cancer Biother Radiopharm 2001; 16:525-35; PMID:11789029.

“Diabodies” as used in context of antibodies, typically refer to bivalent molecules composed of two chains, each comprising a VH and VL domain, either from the same or from different antibodies. The two chains typically have the configuration VHA-VLB and VHB-VLA (A and B representing two different specificities) or VLA-VHB and VLB-VHA.

In context of the present invention, “Diabodies (Db)” or the “Diabody Format” herein refers to bivalent molecules composed of two polypeptide chains, each comprising two variable domains connected by a linker (L_{Db1} and L_{Db2}), wherein two of the domains are first and second domains as defined in context of the present invention (V₁ and V₂) and the other two domains may be TCR derived or antibody derived variable domains (V_{A},V_{B}). The V₁ and V₂ domains are located on two different polypeptides, the VA and VB domains are located on two different polypeptides and the domains dimerize in a head-to-tail orientation. Accordingly, the orientation may be V₁-L_{Db1}-V_{A} and V_{B}-L_{Db2}-V₂, V₂-L_{Db1}-V_{A} and V_{B}-L_{Db2}-V₁, V₁-L_{Db1}-V_{B} and V_{A}-L_{Db2}-V₂ or V₂-L_{Db1}-V_{B} and V_{A}-L_{Db2}-V₁. In order to allow the domains to dimerize head to-tail the linker, i.e. L_{Db1} and L_{Db1},—which are identical or different—are short linkers. A short linker is typically between 2 to 12, 3 to 13, such as 3, 4, 5, 6, 7, 8, 9 amino acids long, for example 4, 5 (Brinkmann U. and Kontermann E. E. (MAbs. 2017 February-March; 9(2): 182-212) or 8 amino acids long, such as ‘GGGS’ of SEQ ID NO: 70, ‘GGGGS’ of SEQ ID NO: 71 or ‘GGGSGGGG’ of SEQ ID NO: 64.

The “Dual-Variable-Domain Immunoglobulin (DVD-Ig™)” format was initially described in 2007 by Wu C. et al. (Nat Biotechnol. 2007 November; 25(11):1290-7). In this format, the target-binding variable domains of a second monoclonal antibody (B) are typically fused to a conventional antibody (A) (comprising the domains V_{LA} and V_{HA}), wherein the light chain of the conventional antibody (A) thus, comprises an additional light chain variable domain (V_{LB}) and the heavy chain of the conventional antibody (A) comprises an additional heavy chain variable domain (V_{HB}). The DVD-Ig™ as described in the art, is thus, typically composed of two polypeptide chains, one heavy chain comprising V_{HB}-L-V_{HA}-C_{H1}-C_{H2}-C_{H3} and one light chain comprising V_{LB}-L-V_{LA}-C_{L}. The domains V_{LA}/V_{HA} and V_{LA}/V_{LA} are thus, pairing in parallel.

In context of the present invention, the “Dual-Variable-Domain Ig Format” refers to a protein comprising two polypeptide chains, each comprising two variable domains connected by a linker (L₁, L₃), wherein two of the domains are first and second domains as defined in context of the present invention (V₁ and V₂) and the other two domains are antibody derived heavy and light chain variable domains (V_{HA} and V_{HB}). In the DVD-Ig format in context of the present invention the polypeptide chains have, for example the organization V₁-L₁-V_{HA}-L₂-C_{H1}-C_{H2}-C_{H3} and V₂-L₃-V_{LA}-L₄-C_{L} or V₂-L₁-V_{HA}-L₂-C_{H1}-C_{H2}-C_{H3} and V₁-L₃-V_{LA}-L₄-C_{L}. The connecting linkers L1 and L₃ are preferably between 5 to 20 residues, such as 5 to 15 amino acids, and/or the connecting linkers L₂ and L₄ may be present or absent.

The “Crossover Dual-Variable Domain-Ig-Like Proteins” as described in the art in context of antibodies represents a format in which two V_{H} and two V_{L} domains are linked in a way that allows crossover pairing of the variable V_{H}-V_{L} domains, which are arranged either (from N- to C-terminus) in the order V_{HA}-V_{HB} and V_{LB}-V_{LA}, or in the order V_{HB}-V_{HA} and V_{LA}-V_{LB}.

In context of the present invention, the “Crossover Dual-Variable Domain-Ig-Like Protein” refer to a protein comprising two polypeptide chains, each comprising two variable domains connected by a linker (L₁, L₂, L₃ and L₄), wherein two of the domains are first and second domains as defined in context of the present invention (V₁ and V₂) and the other two domains are antibody derived heavy and light chain variable domains (V_{HA}, V_{HB}). In the CDVD-Ig format in context of the present invention the polypeptide chains have, for example the organization V₁-L₁-V_{HA}-L₂-C_{H1}-C_{H2}-CH₃ and V_{LA}-L₃-V₂-L₄-C_{L}, V₂-L₁-V_{HA}-L₂-C_{H1}-C_{H2}-C_{H3} and V_{LA}-L₃-V₁-L₄-CL, VHA-L₁-V₁-L₂-C_{H1}-C_{H2}-CH_{H3} and V₂-L₃-V_{LA}-L_{DVD3}-C_{L} or V_{HA}-L₁-V₂-L₂-C_{H1}-C_{H2}-CH₃ and V₁-L₃-V_{LA}-L₄-C_{L}. In this CDVD format, the linkers (L₁ to L₄) are typically of different length, including all-glycine linkers and linkers as described herein below in the section linkers. For example, L₁ is 3 to 12 amino acid residues in length, L₂ is 3 to 14 amino acid residues in length, L₃ is 1 to 8 amino acid residues in length, and L₄ is 1 to 3 amino acid residues in length, or L₁ is 5 to 10 amino acid residues in length, L₂ is 5 to 8 amino acid residues in length, L₃ is 1 to 5 amino acid residues in length, and L₄ is 1 to 2 amino acid residues in length or L₁ is 7 amino acid residues in length, L₂ is 5 amino acid residues in length, L₃ is 1 amino acid residues in length, and L₄ is 2 amino acid residues in length.

An “Antibody Format” herein refers to a protein, wherein further domains that might be comprised, such as a constant domain, derive preferably from an antibody. Referring to the above indicated example, an antigen binding protein comprising a first polypeptide with Valpha-L-V_{L}(CD3)-F_{c} and a second polypeptide with V_{H}(CD₃)-L-Vbeta-F_{c} might be referred to as an antibody format since the F_{c} domain derives from antibodies.

Analogous to BsAb, a “Bispecific TCR” in context of the present invention denotes a a bispecific protein in a TCR format, which combines one antigen-binding site comprising TCR CDRs and one antigen binding site comprising antibody CDRs within a single molecule.

A “TCR Format” herein refers to a protein, wherein further domains that might be comprised, such as a constant domain, derive preferably from a TCR. However, exceptions are possible since a scTCR might also comprise constant domains or fragments thereof of antibody.

The “Antibody Format” is not always clearly distinguishable from a “TCR format” if the number of TCR derived domains equals the number of antibody derived domains, as it is the case, for instance, for an antigen binding protein comprising a first polypeptide with Valpha-L-V_{L}(CD3) and a second polypeptide with V_{H}(CD3)-L-Vbeta, in this special case the antigen binding protein might be considered as being in a TCR format as well as in an antibody format.

“At Least One” herein refers to one or more of the specified objects such as 1, 2, 3, 4, 5 or 6 or more of the specified objects. For example, at least one binding site herein refers to 1, 2, 3, 4, 5 or 6 or more binding sites.

A sequence “At Least 85% Identical to a Reference Sequence” is a sequence having, over its entire length, 85%, or more, in particular 90%, 91%, 92%, 93%, 94%, 95%, 96%, 97%, 98% or 99% sequence identity with the entire length of a reference sequence.

In the context of the present application, the “Percentage of Identity” is calculated using a global pairwise alignment (i.e. the two sequences are compared over their entire length). Methods for comparing the identity of two or more sequences are well known in the art. The «needle» program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk World Wide Web site and is further described in the following publication (EMBOSS: The European Molecular Biology Open Software Suite (2000) Rice, P. Longden, I. and Bleasby, A. Trends in Genetics 16, (6) pp. 276-277). The percentage of identity between two polypeptides, in accordance with the invention, is calculated using the EMBOSS: needle (global) program with a “Gap Open” parameter equal to 10.0, a “Gap Extend” parameter equal to 0.5, and a Blosum62 matrix.

Proteins consisting of an amino acid sequence “At Least 80%, 85%, 90%, 95%, 96%, 97%, 98% or 99% Identical” to a reference sequence may comprise mutations such as deletions, insertions and/or substitutions compared to the reference sequence. In case of substitutions, the protein consisting of an amino acid sequence at least 80%, 85%, 90%, 95%, 96%, 97%, 98% or 99% identical to a reference sequence may correspond to a homologous sequence derived from another species than the reference sequence.

“Amino Acid Substitutions” may be conservative or non-conservative. Preferably, substitutions are conservative substitutions, in which one amino acid is substituted for another amino acid with similar structural and/or chemical properties.

In an embodiment, conservative substitutions may include those, which are described by Dayhoff in “The Atlas of Protein Sequence and Structure. Vol. 5”, Natl. Biomedical Research, the contents of which are incorporated by reference in their entirety. For example, in an aspect, amino acids, which belong to one of the following groups, can be exchanged for one another, thus, constituting a conservative exchange: Group 1: alanine (A), proline (P), glycine (G), asparagine (N), serine (S), threonine (T); Group 2: cysteine (C), serine (S), tyrosine (Y), threonine (T); Group 3: valine (V), isoleucine (I), leucine (L), methionine (M), alanine (A), phenylalanine (F); Group 4:

lysine (K), arginine (R), histidine (H); Group 5: phenylalanine (F), tyrosine (Y), tryptophan (W), histidine (H); and Group 6: aspartic acid (D), glutamic acid (E). In an aspect, a conservative amino acid substitution may be selected from the following of T→A, G→A, A−I, T→V, A→M, T→I, A→V, T→G, and/or T→S.

In a further embodiment, a conservative amino acid substitution may include the substitution of an amino acid by another amino acid of the same class, for example, (1) nonpolar: Ala, Val, Leu, Ile, Pro, Met, Phe, Trp; (2) uncharged polar: Gly, Ser, Thr, Cys, Tyr, Asn, Gln; (3) acidic: Asp, Glu; and (4) basic: Lys, Arg, His. Other conservative amino acid substitutions may also be made as follows: (1) aromatic: Phe, Tyr, His; (2) proton donor: Asn, Gln, Lys, Arg, His, Trp; and (3) proton acceptor: Glu, Asp, Thr, Ser, Tyr, Asn, Gln (see, for example, U.S. Pat. No. 10,106,805, the contents of which are incorporated by reference in their entirety).

In another embodiment, conservative substitutions may be made in accordance with Table 1. Methods for predicting tolerance to protein modification may be found in, for example, Guo et al., Proc. Natl. Acad. Sci., USA, 101(25):9205-9210 (2004), the contents of which are incorporated by reference in their entirety.

TABLE 1
Conservative Amino Acid substitution
Conservative Amino Acid Substitutions

| | Substitutions |
|---|---|
| Amino Acid | (others are known in the art) |
| Ala | Ser, Gly, Cys |
| Arg | Lys, Gln, His |
| Asn | Gln, His, Glu, Asp |
| Asp | Glu, Asn, Gln |
| Cys | Ser, Met, Thr |
| Gln | Asn, Lys, Glu, Asp, Arg |
| Glu | Asp, Asn, Gln |
| Gly | Pro, Ala, Ser |
| His | Asn, Gln, Lys |
| Ile | Leu, Val, Met, Ala |
| Leu | Ile, Val, Met, Ala |
| Lys | Arg, Gln, His |
| Met | Leu, Ile, Val, Ala, Phe |
| Phe | Met, Leu, Tyr, Trp, His |
| Ser | Thr, Cys, Ala |
| Thr | Ser, Val, Ala |
| Trp | Tyr, Phe |
| Tyr | Trp, Phe, His |
| Val | Ile, Leu, Met, Ala, Thr |

In another embodiment, conservative substitutions may be those shown in Table 2 under the heading of “conservative substitutions.” If such substitutions result in a change in biological activity, then more substantial changes, denominated “exemplary substitutions” in Table 2, may be introduced and the products screened if needed.

TABLE 2
Amino Acid substitution
Amino Acid Substitutions

| Original Residue | | |
|---|---|---|
| (naturally occurring | Conservative | |
| amino acid) | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

In some embodiments, the antigen binding protein may include a variant antigen binding protein, wherein said variant antigen binding protein includes a first polypeptide chain (such as a α chain) and a second polypeptide chain (such as a β chain) comprising for up to 8, 9, 10, 11, 12, 13, 14, 15, or more amino acid substitutions, preferably in the CDR regions of the Valpha and Vbeta regions in comparison to the antigen binding protein from which the variant is derived. In this regard, there may be 1, 2, 3, 4, 5, 6, 7, 8, 9, 10, 11, 12, 13, 14, 15 more amino acid substitutions in the CDR regions of the antigen binding protein. Substitutions may be in the CDRs either in the Valpha and/or the Vbeta regions.

High affinity antigen binding proteins may be engineered based on the antigen binding proteins of the invention, with an even stronger binding to PRAME-004 in complex with a MHC than the respective wild type antigen binding protein, which may have affinities with a K_{D} of between about 10⁻⁶ M and about 10⁻¹² M, between about 10⁻⁷ M and about 10⁻¹² M, between about 10⁻⁸ M and about 10⁻¹² M, between about 10⁻⁹ M and about 10⁻¹² M, between about 10⁻¹⁰ M and about 10⁻¹² M, or between about 10⁻¹¹ M and about 10⁻¹² M.

In one embodiment, the variant is a functional variant.

The term “Functional Variant” as used herein refers to an antigen binding protein having substantial or significant sequence identity or similarity to a parent antigen binding protein, such as those antigen binding proteins containing conservative amino acid substitutions, wherein said functional variant retains the biological activity of the parental antigen binding protein. In an aspect, functional variants encompass, for example, those variants of antigen binding proteins described herein (the parent antigen binding proteins) that retain the ability to recognize target cells to a similar extent, the same extent, or to a higher extent, as the parent antigen binding proteins. In reference to the parent antigen binding protein the functional variant can, for instance, have an amino acid sequence that is at least 50%, at least 75%, at least 80%, at least 85%, at least 90%, at least 95%, at least 98%, at least 99% or more identical to the amino acid sequence to the parent antigen binding protein.

The functional variant can, for example, comprise the amino acid sequence of the parent antigen binding protein with at least one conservative amino acid substitution. Alternatively, or additionally, the functional variants can comprise the amino acid sequence of the parent antigen binding protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the antigen binding protein.

The modified TCRs, polypeptides, and proteins of the present disclosure (including functional portions and functional variants) can be of any length, i.e., can comprise any number of amino acids, provided that the modified TCRs, polypeptides, or proteins (or functional portions or functional variants thereof) retain their biological activity, e.g., the ability to specifically bind to an antigen, detect diseased cells in a host, or treat or prevent disease in a host, etc.

The antigen binding proteins of the present inventions (including functional portions and functional variants) can comprise synthetic amino acids in place of one or more naturally-occurring amino acids. Such synthetic amino acids are known in the art, and may include, for example, aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

In one embodiment, the antigen binding protein of the present invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

In a further embodiment, the antigen binding protein of the invention (including functional portions and functional variants) is in the form of a salt, for example, a pharmaceutically acceptable salt. Suitable pharmaceutically acceptable acid addition salts may include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

In this respect, the antigen binding protein of the present disclosure can be synthetic, recombinant, isolated, and/or purified.

The term “Linker” as used herein refers to one or more amino acid residues inserted between domains to provide sufficient mobility for the domains, for example the first variable domain and the second variable domain of the antigen binding antibodies of the invention and, optionally, of the light and heavy chain variable domains, to fold correctly to form the antigen binding site or bispecific antigen binding proteins to form the at least one further antigen binding site, either in a cross over pairing (in a CODV format or in some of the diabody formats) or in a parallel pairing configuration (for example in a DVD format) of the antigen binding proteins.

In some embodiments, a linker consists of 0 amino acid meaning that the linker is absent. A linker is inserted at the transition between variable domains or between variable domains and constant domains, respectively, at the amino acid sequence level. The transition between domains can be identified because the approximate size of the immunoglobulin domains as well as of the TCR domains is well understood. The precise location of a domain transition can be determined by locating peptide stretches that do not form secondary structural elements such as beta-sheets or alpha-helices as demonstrated by experimental data or as can be assumed by techniques of modeling or secondary structure prediction. The term linker used in context of the present invention refers but is not limited to the linkers referred to as L₁, L₂, L₃, L₄, L₅ and L₆.

A linker, as long as it is not specified otherwise in the respective context, such as L₁, L₂, L₃, L₄, L₅ and L₆, can be from at least 1 to 30 amino acids in length. In some embodiments, a linker, such as L₁, L₂, L₃, L₄, L₅ and L₆, can be 2-25, 2-20, or 3-18 amino acids long. In some embodiments, a linker, such as L₁, L₂, L₃, L₄, L₅ and L₆, can be a peptide no more than 14, 13, 12, 11, 10, 9, 8, 7, 6, or 5 amino acids long. In other embodiments, a linker, such as L₁, L2, L₃, L4, L₅ and L₆, can be 5-25, 5-15, 4-11, 10-20, or 20-30 amino acids long. In other embodiments, a linker, such as L₁, L₂, L₃, L₄, L₅ and L₆, can be about, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids long. In a particular, embodiment linkers, such as L₁, L₂, L₃, L₄, L₅ and L₆, may be less than 24, less than 20, less than 16, is less than 12, less than 10, for example from 5 to 24, 10 to 24 or 5-10 amino acid residues in length. In some embodiments, said linker is equal to 1 or more amino acid residues in length, such as more than 1, more than 2, more than 5, more than 10, more than 20 amino acid residues, more than 22 amino acid residues in length.

Exemplary linkers include, for example, the amino acid sequences

(SEQ ID NO: 69)
TVAAP,
(SEQ ID NO: 70)
GGGS,
(SEQ ID NO: 71)
GGGGS,
(SEQ ID NO: 72)
TVLRT,
(SEQ ID NO: 73)
TVSSAS,
(SEQ ID NO: 64)
GGGSGGGG,
(SEQ ID NO: 74)
GGGGSGGGGS,
(SEQ ID NO: 75)
GGGGSAAA,
(SEQ ID NO: 76)
GGGGSGGGGSGGGGS
(SEQ ID NO: 77)
GGGGSGGGGSGGGGSGGGGSGGGGSGS,
(SEQ ID NO: 151)
GGGGSGGGGSGGGGSGGGGSGGGGS,
(SEQ ID NO: 78)
TVLSSAS
and
(SEQ ID NO: 117)
GGGGSGGGGSGGGGSGGGGS,
(SEQ ID NO: 159)
GGGGSGT,
(SEQ ID NO: 160)
GGSGGGGSGG,
(SEQ ID NO: 161)
GGSGGGGSGGGGSGG,
(SEQ ID NO: 162)
GGGGSGGGGSGT
(SEQ ID NO: 163)
GGGGSGGGGSGGGGSGT,
(SEQ ID NO: 164)
GGGGSGGGGSGGGGSGGGGSGT,
(SEQ ID NO: 165)
GGSGGGGSGGGGSGGGGSGG,
(SEQ ID NO: 166)
GGGGSGGGGSGGGGSGGGGSGGGGSGT,
(SEQ ID NO: 167)
GGSGGGGSGGGGSGGGGSGGGGSGG,
(SEQ ID NO: 168)
GSADDAKKDAAKKDGKS,
(SEQ ID NO: 169)
GGQGSGGTGSGGQGSGGTGSGGQGS,
(SEQ ID NO: 170)
GGGGSGGGGSGGGGSGGGGS,

in particular GGGSGGGG (SEQ ID NO: 64), GGGGSGGGGSGGGGSGGGGS (SEQ ID NO: 117) and GGGGSGGGGSGGGGSGGGGSGGGGS (SEQ ID NO: 151).The term “F_{c} Domain” as used in context of the present invention encompasses native F_{c} and F_{c} variants. As with F_{c} variants and native F_{c} molecules, the term “F_{c} domain” includes molecules in monomeric or multimeric form, whether digested from whole antibody or produced by other means.

The term “Native F_{c}” as used herein refers to a molecule comprising the sequence of a non-antigen-binding fragment resulting from digestion of an antibody or produced by other means, whether in monomeric or multimeric form, and can contain the hinge region. The original immunoglobulin source of the native F_{c} is, in particular, of human origin and can be any of the immunoglobulins, although IgG1 and IgG2 are preferred. Native Fc molecules are made up of monomeric polypeptides that can be linked into dimeric or multimeric forms by covalent (i.e., disulfide bonds) and non-covalent association. The number of intermolecular disulfide bonds between monomeric subunits of native F_{c} molecules ranges from 1 to 4 depending on class (e.g., IgG, IgA, and IgE) or subclass (e.g., IgG1, IgG2, IgG3, IgAI, and IgGA2). One example of a native F_{c} is a disulfide-bonded dimer resulting from papain digestion of an IgG. The term “native F_{c}” as used herein is generic to the monomeric, dimeric, and multimeric forms. One example of a native F_{c} amino acid sequence SEQ ID NO: 79.

The “Hinge” or “Hinge Region” or “Hinge Domain” refers typically to the flexible portion of a heavy chain located between the C_{H1} domain and the C_{H2} domain. It is approximately 25 amino acids long, and is divided into an “upper hinge,” a “middle hinge” or “core hinge,” and a “lower hinge.” A “hinge subdomain” refers to the upper hinge, middle (or core) hinge or the lower hinge. The amino acids sequences of the hinges of an IgG1, IgG2, IgG3 and IgG4 molecule are indicated herein below:

IgG1:
(SEQ ID NO. 155)
E₂₁₆PKSCDKTHTCPPCPAPELLG
IgG2:
(SEQ ID NO. 156)
E₂₁₆RKCCVECPPCPAPPVAGP
IgG3:
(SEQ ID NO. 157)
ELKTPLGDTTHTCPRCPEPKSCDTPPPCPRCPE₂₁₆PKSCDTPPPCPRCP
APELLG
IgG4:
(SEQ ID NO. 158)
E₂₁₆SKYGPPCPSCPAPEFLG.

In context of the present invention it is referred to amino acid positions in the Fc domain, these amino acid positions or residues are indicated according to the EU numbering system as described, for example in Edelman, G. M. et al., Proc. Natl. Acad. USA, 63, 78-85 (1969).

The term “F_{c} Variant” as used herein refers to a molecule or sequence that is modified from a native F_{c} but still comprises a binding site for the salvage receptor, F_{c}Rn (neonatal F_{c} receptor). Exemplary F_{c} variants, and their interaction with the salvage receptor, are known in the art. Thus, the term “F_{c} variant” can comprise a molecule or sequence that is humanized from a non-human native F_{c}. Furthermore, a native F_{c} comprises regions that can be removed because they provide structural features or biological activity that are not required for the antigen binding proteins of the invention. Thus, the term “F_{c} variant” comprises a molecule or sequence that lacks one or more native F_{c} sites or residues, or in which one or more F_{c} sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an F_{c} receptor other than a salvage receptor, or (7) antibody-dependent cellular cytotoxicity (ADCC).

In one embodiment, the Fc-domain is a human IgG Fc domain, preferably derived from human IgG1, IgG2, IgG3 or IgG4, preferably IgG1 or IgG2, more preferably IgG1.

In some embodiments, when the antigen binding protein contains two F_{c} domains, i.e. in the F_{c}-containing bispecific TCR/mAb diabody format (TCER® format) used in the examples (F_{c1} and Fₐ), the two F_{c} domains are of the same immunoglobulin isotype or isotype subclass. Accordingly, in some embodiments both F_{c1} and F_{c2} are of the IgG1 subclass, or of the IgG2 subclass, or of the IgG3 subclass, or of the IgG4 subclass.

In a preferred embodiment, both F_{c1} and F_{c2} are of the IgG1 subclass, or of the IgG2 subclass, more preferably of the IgG1 subclass.

In some embodiments, the F_{c} regions further comprise the RF and/or “Knob-into-hole” mutation as defined herein above.

The “RF Mutation” generally refers to the mutation of the amino acids HY into RF in the C_{H3} domain of Fc domains, such as the mutation H435R and Y436F in C_{H3} domain as described by Jendeberg, L. et al., (1997, J. Immunological Meth., 201: 25-34) and is described as advantageous for purification purposes as it abolishes binding to protein A. In case the antigen binding protein comprises two F_{c}-domains, the RF mutation may be in one or both, preferably in one Fc-domain.

The “Knob-Into-Hole” or also called “Knob-into-Hole” technology refers to mutations Y3490, T366S, L368A and Y407V (Hole) and S354C and T366W (Knob) both in the C_{H3}-C_{H3} interface to promote heteromultimer formation has been described in patents U.S. Pat. Nos. 5,731,168 and 8,216,805, notably, which are herein incorporated by reference. Those knob-into-hole mutations can be further stabilized by the introduction of additional cysteine amino acid substitutions Y3490 and S354C.

In context of the present invention, the “Knob” mutation is, for example, present in the F_{c} amino acid sequence of SEQ ID NO: 66 and the “Hole” mutation is, for example, present in the F_{c} amino acid sequence of SEQ ID NO: 68.

In some embodiments, the Fc domain of one of the polypeptides, for example F_{c1}, comprises the amino acid substitution T366W (Knob) in its C_{H3} domain and the Fc domain of the other polypeptide, for example F_{c2}, comprises the amino acid substitution T3665, L368A and Y407V (Hole) in its C_{H3} domain, or vice versa.

In some embodiments, the Fc domain of one of the polypeptides, for example F_{c1}, comprises or further comprises the amino acid substitution S354C in its C_{H3} domain and the Fc domain of the other polypeptide, for example F_{c2}, comprises or further comprises the amino acid substitution Y3490 in its C_{H3} domain, or vice versa.

Accordingly, in some embodiments, the Fc domain of one of the polypeptides, for example F_{c1}, comprises the amino acid substitutions S354C and T366W (Knob) in its C_{H3} domain and the Fc domain of the other polypeptide, for example F_{c2}, comprises the amino acid substitution Y3490, T3665, L368A and Y407V (Hole) in its C_{H3} domain, or vice versa.

This set of amino acid substitutions can be further extended by inclusion of the amino acid substitutions K409A on one polypeptide and F405K in the other polypeptide as described by Wei et al. (Structural basis of a novel heterodimeric Fc for bispecific antibody production, Oncotarget. 2017). Accordingly, in some embodiments, the Fc domain of one of the polypeptides, for example F_{c1}, comprises or further comprises the amino acid substitution K409A in its C_{H3} domain and the Fc domain of the other polypeptide, for example F_{c2}, comprises or further the amino acid substitution F405K in its C_{H3} domain, or vice versa.

In some cases artificially introduced cysteine bridges may improve the stability of the antigen binding proteins, optimally without interfering with the binding characteristics of the antigen binding proteins. Such cysteine bridges can further improve heterodimerization.

Further amino acid substitutions, such as charged pair substitutions, have been described in the art, for example in EP 2 970 484 to improve the heterodimerization of the resulting proteins.

Accordingly in one embodiment, the Fc domain of one of the polypeptides, for example F_{c1}, comprises or further comprises the charge pair substitutions E356K, E356R, D356R, or D356K and D399K or D399R, and the Fc domain of the other polypeptide, for example F_{c2}, comprises or further comprises the charge pair substitutions R409D, R409E, K409E, or K409D and N392D, N392E, K392E, or K392D, or vice versa.

In a further embodiment, the Fc domain on one or both, preferably both polypeptide chains can comprise one or more alterations that inhibit Fc gamma receptor (FcyR) binding. Such alterations can include L234A, L235A.

With the inclusion of Fc-parts consisting of hinges, C_{H2} and C_{H3} domains, or parts thereof, into antigen binding proteins, more particularly into bispecific antigen binding proteins the problem of unspecific immobilization of these molecules, induced by Fc:Fc-gamma receptor (FcgR) interactions arose. FcgRs are composed of different cell surface molecules (FcgRI, FcgRIIa, FcgRIIb, FcgRIII) binding with differing affinities to epitopes displayed by Fc-parts of IgG-molecules. As such an unspecific (i.e. not induced by either of the two binding domains of a bispecific molecule) immobilization is unfavorable due to i) influence on pharmacokinetics of a molecule and ii) off-target activation of immune effector cells various Fc-variants and mutations to ablate FcgR-binding have been identified. In this context, Morgan et al. 1995, Immunology (The N-terminal end of the C_{H2} domain of chimeric human IgG1 anti-HLA-DR is necessary for C1q, FcyRI and FcyRIII binding) disclose the exchange of the residues 233-236 of human IgG1 with the corresponding sequence derived from human IgG2, i.e. the residues 233P, 234V and 235A and wherein no amino acid is present at position 236, resulting in abolished FcgRI binding, abolished C1q binding and diminished FcgRIII binding. EP1075496 discloses antibodies and other Fc-containing molecules with variations in the Fc region (such as one or more of 233P, 234V, 235A and no residue or G in position 236 and 327G, 330S and 331S) wherein the recombinant antibody is capable of binding the target molecule without triggering significant complement dependent lysis, or cell mediated destruction of the target.

Accordingly, in some embodiments, the Fc region comprises or further comprise one or more of the amino acids or deletions selected from the group consisting of 233P, 234V, 235A, 236 (No residue) or G, 327G, 330S, 331S, preferably, the Fc region comprises or further comprises the amino acids 233P, 234V, 235A, 236 (No residue) or G and one or more amino acids selected from the group consisting of 327G, 330S, 331S, most preferably, the Fc region comprises or further comprises the amino acids 233P, 234V, 235A, 236 (No residue) and 331S.

In one further embodiment, the Fc domain comprises or further comprises the amino acid substitution N297Q, N297G or N297A, preferably N297Q.

The amino acid substitution “N297Q”, “N297G” or “N297A” refer to amino acid substitutions at position 297 that abrogate the native N-Glycosylation site within the Fc-domain. This amino acid substitution further prevents Fc-gamma-receptor interaction and decreases the variability of the final protein products, i.e. the antigen binding proteins of the present invention, due to sugar residues as described for example in Tao, MH and Morrison, S L (J Immunol. 1989 Oct. 15; 143(8):2595-601.).

In one further embodiment, in particular when no light chain, the Fc domain comprises or further comprises the amino acid substitution S220C. The amino acid substitution “S220C” deletes the cysteine forming the C_{H1}-CL disulfide-bridge.

In some embodiments, the Fc domain comprises or further comprises at least two additional cysteine residues, for example S354C and Y3490 or L2420 and K3340, wherein S354C is in the Fc-domain of one polypeptide, such as F_{c1}, and Y3490 is in the Fc domain of the other polypeptide, such as Fa, to form a heterodimer and/or wherein L2420 and K334C are located in the same Fc-domain, either in the F_{c1} or F_{c2} of one or both polypeptides to form a intradomain C—C bridge.

By “purified” and “isolated” it is meant, when referring to a polypeptide (i.e. the antibody of the invention) or a nucleotide sequence, that the indicated molecule is present in the substantial absence of other biological macromolecules of the same type. The term “purified” as used herein in particular means at least 75%, 85%, 95%, or 98% by weight, of biological macromolecules of the same type are present.

An “isolated” nucleic acid molecule that encodes a particular polypeptide refers to a nucleic acid molecule that is substantially free of other nucleic acid molecules that do not encode the subject polypeptide; however, the molecule may include some additional bases or moieties, which do not deleteriously affect the basic characteristics of the composition.

A “domain” may be any region of a protein, generally defined on the basis of sequence homologies and often related to a specific structural or functional entity.

A “recombinant” molecule is one that has been prepared, expressed, created, or isolated by recombinant means.

The term “gene” means a DNA sequence that codes for, or corresponds to, a particular sequence of amino acids which comprises all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

“Half Maximal Effective Concentration” also called “EC₅₀” typically refers to the concentration of a molecule which induces a response halfway between the baseline and maximum after a specified exposure time. EC₅₀ and affinity are inversely related, the lower the EC₅₀ value the higher the affinity of the molecule. In one example, the “EC₅₀” refers to the concentration of the antigen binding protein of the invention which induces a response halfway between the baseline and maximum after a specified exposure time, more particularly, refers to the concentration of the antigen binding protein of the invention which induces a response halfway between the baseline and maximum after a specified exposure time. EC₅₀ values can be experimentally assessed by a variety of known methods, using for example IFN-gamma release assay or a LDH release assay.

### Antigen Binding Proteins

Using the TCR R16P1C10, as disclosed in WO2018/172533, which is incorporated herein by reference, as starting point, the inventors have designed, produced and tested TCR variable alpha (Valpha) and variable beta (Vbeta) domain variants either in a single-chain (scTCR) format; more exactly in a single chain bispecific TCR, optionally coupled to a Fab-fragment, or in a F_{c}-containing bispecific TCR/mAb diabody format. In this way the inventors identified different variants of the variable alpha and beta domain, more particularly different variants of the two complementary determining regions (CDRs) CDRa1 (with 1 variant) and CDRa3 (with 27 variants) of the variable alpha domain and different variants of the two complementary determining regions (CDRs) CDRb1 (with 2 variants) and CDRb3 (with 1 to 14 variants) of the variable beta domain that are relevant for the antigen binding proteins of the invention to bind the target, i.e. a PRAME peptide comprising the amino acid sequence SLLQHLIGL of SEQ ID NO: 8 in a complex with a MHC protein, preferably in complex with HLA -A*02, with a high affinity but also high specificity. The inventors demonstrated in the examples that the CDRs may be used in single chain TCR constructs as well as in bispecific antibodies and thus demonstrated in a proof of principle that the identified CDR variants may be used to produce different antigen binding proteins having a high affinity but also a high specificity to the PRAME peptide comprising the amino acid sequence SLLQHLIGL of SEQ ID NO: 8 in a complex with a MHC protein, preferably in complex with HLA-A*02. Moreover, the inventors discovered that by mutating the CDRa2 amino acid sequence IYSNGD (SEQ ID NO 9) into the amino acid sequence IYX₂X₃GD (SEQ ID NO: 2), wherein X₂ is any amino acid, preferably S or Q, more preferably Q, X₃ is any amino acid, preferably T, E, D or A, more preferably E, improves the stress stability of the antigen binding proteins of the invention.

In the examples, the inventors designed, for example, F_{c}-containing bispecific TCR/mAb diabodies specifically binding to the human TCR-CD3 complex and to the peptide : MHC complex comprising the PRAME peptide SLLQHLIGL (SEQ ID NO: 8) bound to MHC class I molecule, e.g., HLA-A2*01. For targeting the TCR-CD3 complex, V_{H} and V_{L} domains derived from the CD3-specific, humanized antibody hUCHT1(V₉) described by Zhu et al., (Identification of heavy chain residues in a humanized anti-CD3 antibody important for efficient antigen binding and T cell activation. J Immunol, 1995, 155, 1903-1910) are used in those example, alternatively, V_{H} and V_{L} domains derived from the alpha/beta TCR-specific antibody BMA031 described in Shearman et al. (Construction, expression and characterization of humanized antibodies directed against the human alpha/beta T cell receptor (J Immunol, 1991, 147, 4366-73) and humanized versions thereof may be used. For targeting the PRAME-004:MHC complex, the Valpha and Vbeta domains comprising the CDRs as herein disclosed may be utilized leading to stability and affinity maturated antigen binding proteins. The same V_{L} and V_{H}, Valpha and Vbeta domains were further used in a human single chain TCR further comprising a Fab domain (comprising a C_{H1}-Hinge and C_{L}). The contents of these references are hereby incorporated by reference in their entireties.

Accordingly, in one embodiment, the present invention refers to an antigen binding protein which specifically binds to a PRAME peptide, which PRAME peptide comprises the amino acid sequence SLLQHLIGL of SEQ ID NO: 8 and is in a complex with a MHC protein, the antigen binding protein comprising
(a) a first polypeptide chain comprising a first variable domain comprising three complementary determining regions (CDRs) CDRa1, CDRa2 and CDRa3, wherein
   the CDRa1 comprises or consists of the amino acid sequence DRGSQX₁ (SEQ ID NO: 1), wherein X₁ is any amino acid, preferably L;
   the CDRa2 comprises or consists of the amino acid sequence IYX₂X₃GD (SEQ ID NO: 2), wherein X₂ is any amino acid, preferably S or Q, more preferably Q, X₃ is any amino acid, preferably T, E, D or A, more preferably E, with the proviso that the CDRa2 does not comprise or consist of the amino acid sequence IYSNGD (SEQ ID NO: 9),
   the CDRa3 comprises or consists of the amino acid sequence CAAVIX₄NX₅X₆GGX₇LTF (SEQ ID NO: 3), wherein X₄ to X₇ is any amino acid,
   preferably, X₄ is S, N, D, P, T, or E, more preferably P; X₅ is L, I, V, Q, N, P, S, A, G, T, D, K, R, F, more preferably V; X₆ is E, A, V, R, N, G, Y, I, D, H, L, P, S, Q, more preferably V; X₇ is S, I, Q, A, R, L, P, Y, K, M, more preferably Q; and
(b) a second polypeptide chain comprising a second variable domain comprising three complementary determining regions (CDRs) CDRb1, CDRb2 and CDRb3, wherein the CDRb1 comprises or consists of the amino acid sequence X₈GHRX₉ (SEQ ID NO: 4), wherein X₈ is any amino acid, preferably S or P, more preferably P and X₉ is any amino acid, preferably S or A, more preferably A; and
   the CDRb2 comprises or consists of the amino acid sequence YX₁₀X₁₁X₁₂X₁₃X₁₄ (SEQ ID NO: 5), wherein X₁₀ to X₁₄ is any amino acid, preferably X₁₀ is V, I, F or E, more preferably

I or V, more preferably V; X₁₁ is H, D, or S, more preferably D or H, more preferably H; X₁₂ is G, R or E, more preferably G; X₁₃ is Q, R, A, F, V, Q, E, L, Y or T, more preferably E or A, more preferably E; X₁₄ is E, A or Q, more preferably E or Q, more preferably E, and the CDRb3 comprises or consists of the amino acid sequence CASSPWDSPNX₁₅QYF (SEQ ID NO: 6), wherein X₁₅ is any amino acid preferably V.

As it will be understood by the skilled in the art, the first variable domain comprising the CDR amino acid sequences as herein defined and the second variable domain comprising the CDR amino acid sequences as herein defined form together an antigen binding site, wherein said antigen binding site binds to the PRAME peptide comprising or consisting of the amino acid sequence SLLQHLIGL of SEQ ID NO: 8 complexed with a MHC protein.

The three complementary determining regions (CDRs) CDRa1, CDRa2 and CDRa3 are derived from the variable alpha domain of a TCR, accordingly, as it will be understood by the skilled in the art, in some embodiments, the first variable domain may also be referred to as variable alpha domain. In analogy, the three complementary determining regions (CDRs) CDRb1, CDRb2 and CDRb3 are derived from the variable beta domain of said TCR, and it will thus further be understood by the skilled in the art, that the second variable domain may also be referred to as variable beta domain. More particularly, a first variable domain comprising the alpha variable framework amino acid sequences and the alpha variable CDRs as defined in context of the inventions, such as the sequence (FR1-a)-(CDRa1)-(FR2-a)-(CDRa2)-(FR3-a)-(CDRa3)-(FR4-a) might be referred to as variable alpha domain and/or a second variable domain comprising the beta variable framework amino acid sequences and the beta variable CDRs as defined in context of the inventions, such as the sequence (FR1-b)-(CDRb1)-(FR2-b)-(CDRb2)-(FR3-b)-(CDRb3)-(FR4-b) might be referred to as variable beta domain.

In some embodiments, the CDRs may be engrafted in an antibody framework sequence. Accordingly, in one example, the alpha variable domain CDRs might be engrafted in the light chain variable domain, the first variable domain thus comprises or consists of the sequences (FR1-L)-(CDRa1)-(FR2-L)-(CDRa2)-(FR3-L)-(CDRa3)-(FR4-L) and might be referred to as a light chain variable domain. In the same example, the beta variable domain CDRs might be engrafted in the heavy chain variable domain, the second variable domain thus comprises or consists of the sequences (FR1-H)-(CDRb1)-(FR2-H)—(CDRb2)-(FR3-H)—(CDRb3)-(FR4-H) and might be referred to as a heavy chain variable domain.

In one embodiment, the CDRa1 comprises or consists of the amino acid sequence DRGSQX₁ (SEQ ID NO: 1), wherein X₁ is any amino acid, except S, and/or
the CDRb1 comprises or consists of the amino acid sequence X₈GHRX₉ (SEQ ID NO: 4), with the proviso that X₈ and X₉ are not both S, and/or
the CDRb3 comprises or consists of the amino acid sequence CASSPWDSPNX₁₅QYF (SEQ ID NO: 6), wherein X₁₅ is any amino acid, except E.

In one embodiment, the CDRa1 comprises or consists of the amino acid sequence DRGSQX, (SEQ ID NO: 1), wherein X₁ is any amino acid selected from the group consisting of A, R, N, D, C, Q, E, G, H, I, L, K, M, F, P, T, W, Y or V, preferably L, and/or
the CDRb1 comprises or consists of the amino acid sequence X₈GHRX₉ (SEQ ID NO: 4), with the proviso that X₈ and X₉ are any amino acid, same or different, selected from the group consisting of A, R, N, D, C, Q, G, E, H, I, L, K, M, F, P, T, W, Y or V , and/or
the CDRb3 comprises or consists of the amino acid sequence CASSPWDSPNX₁₅QYF (SEQ ID NO: 6), wherein X₁₅ is any amino acid selected from the group consisting of A, R, N, D, C, Q, G, H, I, L, K, M, F, P, S, T, W, Y or V.

In context of the present invention, the CDRs of the antigen binding proteins of the invention differ from the CDRs of the parental TCR R16P1010, as disclosed in WO2018/172533, at least in the amino acid sequence of CDRa3 of said parental TCR R16P1010. As it will be understood by the skilled in the art, the CDRa3, as defined herein above, does not comprise or consist of the amino acid sequence ‘CAAVISNFGNEKLTF’ (SEQ ID NO: 10) which is the CDRa3 of the parental TCR R16P1C10.

Moreover, in some embodiments, the CDRa1 does not comprise or consist of the amino acid sequence DRGSQS (SEQ ID NO: 11), and/or CDRb1 does not comprise or consist of the amino acid sequence SGHRS (SEQ ID NO: 12) and/or, CDRb3 does not comprise or consist of the amino acid sequence CASSPWDSPNEQYF (SEQ ID NO: 13).

In one embodiment, said second variable domain comprises a CDRb2 comprising or consisting of the amino acid sequence YX₁₀X₁₁X₁₂X₁₃X₁₄ (SEQ ID NO: 5), with the proviso that CDRb2 does not comprise or consist of the amino acid sequence ‘YFSETQ’ (SEQ ID NO: 14).

In a further embodiment, said second variable domain further comprises a CDRb2 comprising or consisting of the amino acid sequence YVHGX₁₆E (SEQ ID NO: 15), wherein X₁₆ is any amino acid, preferably X₁₆ is Q, R, A, F, V, Q, E, L, Y or T, preferably E.

In one embodiment, the first variable domain and the second variable domain as herein defined may comprise an amino acid substitution at position 44 according to the IMGT numbering. In a preferred embodiment, said amino acid at position 44 is substituted with another suitable amino acid, in order to improve pairing. In particular embodiments, in which said antigen binding protein is a TCR, said mutation improves for example the pairing of the chains (i.e. pairing of α and β chains or paring of γ and δ). In one embodiment, one of or both of the amino acids present at position 44 in the first variable domain (v₁44) and the amino acid present at position 44 in the second variable domain (v₂44) are substituted into the amino acid pairs v₁44/v₂44 selected from the group of amino acid pairs consisting of v₁44D/v₂44R, ᵥ₁44R/v₂44D, ᵥ₁44E/v₂44K, v₁44K/v₂44E, v₁44D/v₂44K, v₁44K/v₂44D, v₁44R/v₂44E; v₁44E/v₂44R, v₁44Uv₂44W, v₁44W/v₂44L, v₁44V/v₂44W, v₁44W/v₂44V.

Accordingly, in a further embodiment, the antigen binding protein may further include one of the preferred substitution pairs (v₁44/v₂44) selected from the group consisting of: v₁Q44D/v₂Q44R; v₁Q44R/v₂Q44D; v₁Q44E/v₂Q44K; vᵢQ44K/v₂Q44E; v₁Q44D/v₂Q44K; v₁Q44K/v₂Q44D; v₁Q44E/v₂Q44R; v₁Q44R/v₂Q44E; v₁Q44L/v₂Q44W; v₁Q44W/v₂Q44L; v₁Q44V/v₂Q44W; and v₁Q44W/v₂Q44V; v₁W44D/v₂Q44R; v₁W44R/v₂Q44D; v₁W44E/v₂Q44K; v₁W44K/v₂Q44E; v₁W44D/v₂Q44K; v₁W44K/v₂Q44D; v₁W44E/v₂Q44R; v₁W44R/v₂Q44E; v₁W44L/v₂Q44W; v₁W44/v₂Q44L; v₁ W44V/v₂Q44W; and v₁ W44/v₂Q44V; v₁ H44D/v₂Q44R; v₁H44R/v₂Q44D; v₁H44E/v₂Q44K; v₁ H44K/v₂Q44E; v₁H44D/v₂Q44K; v₁ H44K/v₂Q44D; v₁H144E/v₂Q44R; v₁H144R/v₂Q44E; v₁H44W/v₂Q44W; v₁H44W/v₂Q44L; v₁H144V/v₂Q44W; and v₁H44W/v₂Q44V; v₁K44D/v₂Q44R; v₁K44R/v₂Q44D; v₁K44E/v₂Q44K; v₁K44/v₂Q44E; v₁K44D/v₂Q44K; v₁K44/v₂Q44D; v₁K44E/v₂Q44R; v₁K44R/v₂Q44E; v₁K44L/v₂Q44W; v₁K44W/v₂Q44L; v₁K44V/v₂Q44W; and v₁K44W/v₂Q44V; v₁E44D/v₂Q44R; v₁E44R/v₂Q44D; v₁E44/v₂Q44K; v₁E44K/v₂Q44E; v₁E44D/v₂Q44K; v₁E44K/v₂Q44D; v₁E44/v₂Q44R; v₁E44R/v₂Q44E; v₁E44L/v₂Q44W; v₁E44W/v₂Q44L; v₁E44V/v₂Q44W; and v₁E44W/v₂Q44V; v₁Q44D/v₂R44; v₁Q44R/v₂R44D; v₁Q44E/v₂R44K; v₁Q44K/v₂R44E; v₁Q44D/v₂R44K; v₁Q44K/v₂R44D; v₁Q44E/v₂R44; v₁Q44R/v₂R44E; v₁Q44L/v₂R44W; v₁Q44W/v₂R44L; v₁Q44V/v₂R44W; and v₁Q44W/v₂R44V; v₁W44D/v₂R44; v₁W44R/v₂R44D; v₁W44E/v₂R44K; v₁W44K/v₂R44E; v₁W44D/v₂R44K; v₁W44K/v₂R44D; v₁W44E/v₂R44; v₁W44R/v₂R44E; v₁W44L/v₂R44W; v₁W44/v₂R44L; v₁W44V/v₂R44W; and v₁W44/v₂R44V; v₁ H44D/v₂R44; v₁H44R/v₂R44D; v1H44E/v₂R44K; v₁H44K/v₂R44E; v₁ H44D/v₂R44K; v₁H44K/v₂R44D; v₁H44E/v₂R44; v₁H44R/v₂R44E; v₁H44L/v₂R44W; v₁ H44W/v₂R44L; v₁H44V/v₂R44W; and v₁H44W/v₂R44V; v₁K44D/v₂R44; v₁K44R/v₂R44D; v₁K44E/v₂R44K; v₁K44/v₂R44E; v₁K44D/v₂R44K; v₁K44/v₂R44D; v₁K44E/v₂R44; v₁K44R/v₂R44E; v₁K44L/v₂R44W; v₁K44W/v₂R44L; v1K44V/v₂R44W; and v₁K44W/v₂R44V; v₁E44D/v₂R44; v₁E44R/v₂R44D; v₁E44/v₂R44K; v₁E44K/v₂R44E; v₁E44D/v₂R44K; v₁E44K/v₂R44D; v₁E44R/v₂R44E; v₁E44L/v₂R44W; v₁E44W/v₂R44L; v₁E44V/v₂R44W; and v₁E44W/v₂R44V; v₁Q44D/v₂K44R; v₁Q44R/v₂K44D; v₁Q44E/v₂44K; v₁Q44K/v₂K44E; v₁Q44D/v₂44K; v₁Q44K/v₂K44D; v₁Q44E/v₂K44R; v₁Q44R/v₂K44E; v₁Q44L/v₂K44W; v₁Q44W/v₂K44L; v₁Q44V/v₂K44W; and v₁Q44W/v2K44V; v₁W44D/v₂K44R; v₁W44R/v₂K44D; v₁W44E/v₂44K; v₁W44K/v₂K44E; v₁W44D/v₂44K; v₁W44K/v₂K44D; v₁W44E/v₂K44R; v₁W44R/v₂K44E; v₁W44Uv₂K44W; v₁W44/v₂K44L; v₁W44V/v₂K44W; and v₁W44/v₂K44V; v₁H144D/v₂K44R; v₁H44R/v₂K44D; v₁H44E/v₂44K; v₁H44K/v₂K44E; v₁H44D/v₂44K; v₁H44K/v₂K44D; v₁H44E/v₂K44R; v₁H44R/v₂K44E; v₁H44L/v₂K44W; v₁H44W/v₂K44L; v₁H44V/v₂K44W; and v₁H44W/v₂K44V; v₁K44D/v₂K44R; v K44R/v₂K44D; v K44E/v₂44K; v₁K44/v₂K44E; v₁ K44D/v₂44K; v₁K44/v₂K44D; v₁K44E/v₂K44R; v₁K44R/v2K44E; v₁K44L/v₂K44W; v₁K44W/v₂K44L; v₁K44V/v₂K44W; and v₁K44W/v₂K44V; v₁E44D/v₂K44R; v₁E44R/v₂K44D; v₁E44/v₂44K; v₁E44K/v₂K44E; v₁E44D/v₂44K; v₁E44K/v₂K44D; v₁E44/v₂K44R; v₁E44R/v₂K44E; v₁E44L/v₂K44W; v₁E44W/v₂K44L; v₁E44V/v₂K44W; and v₁E44W/v₂Q44V.

In the above, e.g., “v₁Q44R/v₂Q44D” shall mean, for example, that, in the first variable domain Q44 is substituted by R, while in the second variable domain, Q44 is substituted by D. Additional substitutions and description may be found in U.S. Patent Application No. 2018-0162922.

In a preferred embodiment, the amino acid as present at position 44 in the variable domain is substituted by one amino acid selected from the group consisting of Q, R, D, E, K, L, W, and V.

Accordingly, in a further preferred embodiment, the antigen binding protein may further include one of the preferred substitution pairs selected from the following lists: αQ44D/βQ44R; αQ44R/βQ44D; αQ44E/βQ44K; αQ44K/βQ44E; αQ44D/βQ44K; αQ44K/βQ44D; αQ44E/βQ44R; αQ44R/βQ44E; αQ44L/Q44W; αQ44W/βQ44L; αQ44V/βQ44W; and αQ44W/βQ44V; αW44D/βQ44R; αW44R/βQ44D; αW44E/βQ44K; αW44K/βQ44E; αW44D/βQ44K; αW44K/βQ44D; αW44E/βQ44R; αW44R/βQ44E; αW44L/βQ44W; αW44/βQ44L; αW44V/βQ44W; and αW44/βQ44V; αH44D/βQ44R; αH44R/βQ44D; αH44E/βQ44K; αH44K/βQ44E; αH44D/βQ44K; αH44K/βQ44D; αH44E/βQ44R; αH44R/βQ44E; αH44L/βQ44W; αH44W/βQ44L; αH44V/βQ44W; and αH44W/βQ44V; αK44D/βQ44R; αK44R/βQ44D; αK44E/βQ44K; αK44/βQ44E; αK44D/βQ44K; αK44/βQ44D; αK44E/βQ44R; αK44R/βQ44E; αK44L/βQ44W; αK44W/βQ44L; αK44V/βQ44W; and αK44W/βQ44V; αE44D/βQ44R; αE44R/βQ44D; αE44/βQ44K; αE44K/βQ44E; αE44D/βQ44K; αE44K/βQ44D; αE44/βQ44R; αE44R/βQ44E; αE44L/Q44W; αE44W/βQ44L; αE44V/βQ44W; and αE44W/βQ44V; αQ44D/βR44; αQ44R/βR44D; αQ44E/βR44K; αQ44K/βR44E; αQ44D/βR44K; αQ44K/βR44D; αQ44E/βR44; αQ44R/βR44E; αQ44L/βR44W; αQ44W/βR44L; αQ44V/βR44W; and αQ44W/βR44V; αW44D/βR44; αW44R/βR44D; αW44E/βR44K; αW44K/βR44E; αW44D/βR44K; αW44K/βR44D; αW44E/βR44; αW44R/βR44E; αW44L/βR44W; αW44/βR44L; αW44V/βR44W; and αW44/βR44V; αH44D/βR44; αH44R/βR44D; αH44E/βR44K; αH44K/βR44E; αH44D/βR44K; αH44K/βR44D; αH44E/βR44; αH44R/βR44E; αH44L/βR44W; αH44W/βR44L; αH44V/βR44W; and αH44W/βR44V; αK44D/βR44; αK44R/βR44D; αK44E/βR44K; αK44/βR44E; αK44D/βR44K; αK44/βR44D; αK44E/βR44; αK44R/βR44E; αK44L/βR44W; αK44W/βR44L; αK44V/βR44W; and αK44W/βR44V; αE44D/βR44; αE44R/βR44D; αE44/βR44K; αE44K/βR44E; αE44D/βR44K; αE44K/βR44D; αE44R/βR44E; αE44L/βR44W; αE44W/βR44L; αE44V/βR44W; and αE44W/βR44V.

In the above, e.g., “αQ44R/βQ44D” shall mean, for example, that, in the first variable domain Q44 is substituted by R, while in the second variable domain, Q44 is substituted by D. Additional substitutions and description may be found in U.S. Patent Application No. 2018-0162922, the contents of which are herein incorporated by reference in their entirety.

In one embodiment, said antigen binding protein specifically binds to the PRAME peptide comprising the amino acid sequence of SEQ ID NO: 8 in a complex with a MHC protein, in particular a MHC class I HLA protein, such as HLA-A, HLA-B, HLA-C, more preferably a HLA-A*02 protein.

MHC proteins, in particular a MHC class I and HLA-A*02 proteins are as defined herein above in the section ‘Definitions’.

In one embodiment, the first variable domain of the antigen binding proteins of the invention comprises:

a CDRa1 comprising or consisting of the amino acid sequence selected from the group consisting of the amino acid sequences DRGSQS (SEQ ID NO: 11) and DRGSQL (SEQ ID NO: 16), and/or

a CDRa2 comprising or consisting of the amino acid sequence selected from the group consisting of the amino acid sequences IYSNGD (SEQ ID NO: 9) and IYQEGD (SEQ ID NO: 17) and/or

a CDRa3 comprising or consisting of the amino acid sequence selected from the group consisting of the amino acid sequences CAAVINNPSGGMLTF (SEQ ID NO: 18), CAAVIDNSNGGILTF (SEQ ID NO: 19), CAAVIDNPSGGILTF (SEQ ID NO: 20), CAAVIDNDQGGILTF (SEQ ID NO: 21), CAAVIPNPPGGKLTF (SEQ ID NO: 22), CAAVIPNPGGGALTF (SEQ ID NO: 23), CAAVIPNSAGGRLTF (SEQ ID NO: 24), CAAVIPNLEGGSLTF (SEQ ID NO: 25), CAAVIPNRLGGYLTF (SEQ ID NO: 26), CAAVIPNTDGGRLTF (SEQ ID NO: 27), CAAVIPNQRGGALTF (SEQ ID NO: 28), CAAVIPNVVGGILTF (SEQ ID NO: 29), CAAVITNIAGGSLTF (SEQ ID NO: 30), CAAVIPNNDGGYLTF (SEQ ID NO: 31), CAAVIPNGRGGLLTF (SEQ ID NO: 32), CAAVIPNTHGGPLTF (SEQ ID NO: 33), CAAVIPNDVGGSLTF (SEQ ID NO: 34), CAAVIENKPGGPLTF (SEQ ID NO: 35), CAAVIDNPVGGPLTF (SEQ ID NO: 36), CAAVIPNNNGGALTF (SEQ ID NO: 37), CAAVIPNDQGGILTF (SEQ ID NO: 38), CAAVIPNVVGGQLTF (SEQ ID NO: 39), CAAVIPNSYGGLLTF (SEQ ID NO: 40), CAAVIPNDDGGLLTF (SEQ ID NO: 41), CAAVIPNAAGGLLTF (SEQ ID NO: 42), CAAVIPNTIGGLLTF (SEQ ID NO: 43) and CAAVIPNTRGGLLTF (SEQ ID NO: 44), and the second variable domain comprises:

a CDRb1 comprising or consisting of the amino acid sequence selected from the group consisting of the amino acid sequences SGHRS (SEQ ID NO: 12) and PGHRA (SEQ ID NO: 45) and/or

a CDRb2 comprising or consisting of the amino acid sequence selected from the group consisting of the amino acid sequences YFSETQ (SEQ ID NO: 14), YVHGEE (SEQ ID NO: 46) and YVHGAE (SEQ ID NO: 47) and/or

a CDRb3 comprising or consisting of the amino acid sequence selected from the group consisting of the amino acid sequences CASSPWDSPNEQYF (SEQ ID NO: 13) and CASSPWDSPNVQYF (SEQ ID NO: 48).

The inventors of the present invention identified in the examples, in particular examples 1 to 4 as herein disclosed, the TCR variant “HiAff1” and “LoAff3” of which the CDR amino acid sequences, when used in the antigen binding proteins of the invention, in particular in bispecific antigen binding proteins, more particularly in a F_{c}-containing bispecific TCR/mAb (anti-CD3) diabody format, increase the binding affinity, the stability and the specificity of the antigen binding proteins comprising those CDRs, in particular, in comparison to a reference protein. Such a reference protein may be, for example, an antigen binding protein comprising the

CDRs of the parental/wild type TCR R16P1010, which is disclosed in WO2018/172533, for instance, a F_{c}-containing bispecific TCR/mAb (anti-CD3) diabody as herein described comprising the CDRs of said TCR R16P1C10 or the reference protein is an antigen binding protein comprising the CDRs of said TCR R16P1010 and is in the same format as the antigen binding protein with which it is compared. Such a reference protein may also be, for example, an antigen binding protein comprising the CDRs of “CDR6”, for instance, a F_{c}-containing bispecific TCR/mAb (anti-CD3) diabody as herein described comprising the CDRs of “CDR6” or the reference protein is an antigen binding protein comprising the CDRs of “CDR6” and is in the same format as the antigen binding protein with which it is compared, wherein the CDRs of “CDR6” are disclosed herein above.

The inventors demonstrated furthermore that the antigen binding proteins of the invention comprising the above described CDRs have an improved stability in comparison to an antigen binding protein comprising the CDRs of a reference antigen binding protein called “CDR6”, wherein the antigen binding protein called “CDR6” comprises the following alpha and beta CDRs: CDRa1 comprising or consisting of the amino acid sequence DRGSQS (SEQ ID NO: 11), and CDRa2 comprising or consisting of the amino acid sequence IYSNGD (SEQ ID NO: 9), and CDRa3 comprising or consisting of the amino acid sequence CAAVIDNDQGGILTF (SEQ ID NO: 21), and CDRb1 comprising or consisting of the amino acid sequence PGHRA (SEQ ID NO: 45), and CDRb2 comprising or consisting of the amino acid sequence YVHGEE (SEQ ID NO: 46), and CDRb3 comprising or consisting of the amino acid sequence CASSPWDSPNVQYF (SEQ ID NO: 48).

In one particular embodiment the invention refers to antigen binding proteins comprising the CDRs of the so-called “HiAff #1” and “LoAff #3” variants and variants thereof. Accordingly, in one preferred embodiment, the antigen binding protein of the invention comprises
a) a first polypeptide chain comprising a first variable domain comprising three complementary determining regions (CDRs) CDRa1, CDRa2 and CDRa3, wherein the CDRa1 comprises or consists of the amino acid sequence DRGSQS (SEQ ID NO: 11) or an amino acid sequence at least 85% identical to SEQ ID NO: 11,
   the CDRa2 comprises or consists of the amino acid sequence IYQEGD (SEQ ID NO: 17) and
   the CDRa3 comprises or consists of the amino acid sequence CAAVIDNDQGGILTF (SEQ ID NO: 21), and
b) a second polypeptide chain comprising a second variable domain comprising three complementary determining regions (CDRs) CDRb1,CDR2b and CDRb3, wherein the CDRb1 comprises or consists of the amino acid sequence PGHRA (SEQ ID NO: 45) or PGHRS (SEQ ID NO: 49), preferably PGHRA (SEQ ID NO: 45), or an amino acid sequence at least 85% identical to SEQ ID NO: 45 or SEQ ID NO: 49, preferably SEQ ID NO: 45;
   the CDRb2 comprises or consists of the amino acid sequence YVHGEE (SEQ ID NO: 46) or an amino acid sequence at least 85% identical to SEQ ID NO: 46, and
   the CDRb3 comprises or consists of the amino acid sequence CASSPWDSPNEQYF (SEQ ID NO: 13) or CASSPWDSPNVQYF (SEQ ID NO: 48), preferably CASSPWDSPNVQYF (SEQ ID NO: 48), or an amino acid sequence at least 85% identical to SEQ ID NO: 13 or SEQ ID NO: 48, preferably CASSPWDSPNVQYF (SEQ ID NO: 48).

As it can be seen from the examples, for instance example 4, the antigen binding proteins of the invention, for instance, in the form of a F_{c}-containing bispecific TCR/mAb diabody, have an increased affinity in comparison to a reference protein, such as an antigen binding protein in the form of a F_{c}-containing bispecific TCR/mAb diabody comprising the CDRs of the TCR R16P1010 with the exception that CDRb1 contains the stabilizing mutation S27P to improve expression in CHO cells.

Accordingly, in one embodiment, the antigen binding proteins of the invention have an increased affinity, optionally, in comparison to a reference protein.

A “Reference Protein” herein refers to a protein to which the antigen binding protein of the invention is compared. In one embodiment, the reference protein is in same format as the antigen binding protein to which it is compared and/or said reference protein comprises the CDRs or the alpha and beta variable domains of the parental TCR R16P1010.

In one embodiment, the antigen binding protein of the invention binds to a complex of the PRAME peptide comprising or consisting of the amino acid sequence of SEQ ID NO: 8 and a HLA molecule, preferably HLA-A*02, with a K_{D} which is ≤200 nM, ≤150 nM, ≤120 nM, 110 nM preferably ≤100 nM, for instance 10pM to 200 nM, 10 pM to 150 nM, 10 pM to 100 nM, in particular 50 pM to 100 nM, 100 pM to 100 nM, 1 nM to 100 nM.

Accordingly, the antigen binding protein of the invention has an affinity (K_{D}) for a complex of the PRAME peptide comprising or consisting of the amino acid sequence of SEQ ID NO: 8 and a HLA molecule, preferably HLA-A*02, which is ≤200 nM, ≤150 nM, ≤120 nM, 110 nM, preferably ≤100 nM, in particular 50 pM to 100 nM, 100 pM to 100 nM, 1 nM to 100 nM. “K_{D}” and “affinity” are as defined herein above in the section definitions.

In one related embodiment, the antigen binding protein is preferably a diabody, optionally, a diabody comprising an F_{c} domain, more particularly a bispecific diabody, optionally, comprising an F_{c} domain.

Methods to measure the affinity, such as the K_{D} are known to the skilled in the art and include, for example, surface Plasmon resonance and biolayer interferometry. As it is known to the skilled in the art the experimental conditions used for those experiments, such as buffer used, concentration of the protein, can strongly influence the results.

Accordingly, in one example, the antigen binding proteins of the invention are expressed, for instance, as soluble F_{c}-containing bispecific TCR/mAb diabody and are analyzed for their binding affinity towards the complex HLA-A*02/PRAME-004 monomers. Typically, measurements are performed, for instance, on an Octet RED384 system using, typically, settings recommended by the manufacturer. Briefly, binding kinetics were, typically, measured at 30° C. and, for instance, 1000 rpm shake speed using, for example, PBS, 0.05% Tween-20, 0.1% BSA as buffer. The antigen binding proteins, in particular, the F_{c}-containing bispecific TCR/mAb diabody were loaded onto biosensors, such as FAB2G or AHC, prior to analyzing serial dilutions of the HLA-A*02/PRAME-004 complex.

As disclosed herein, the antigen binding proteins of the invention recognize and/or specifically binds to a complex of the PRAME peptide comprising or consisting of the amino acid sequence of SEQ ID NO: 8 and a HLA molecule, preferably HLA-A*02. The PRAME-004 and the HLA molecule are thus present in a major histocompatibility complex (MHC) class I, and, the binding of the antigen binding protein to said complex may elicit an immune response upon binding.

Accordingly, in one embodiment, the antigen binding protein of the present invention, induces an immune response, preferably, wherein the immune response is characterized by an increase in interferon gamma (IFNγ) levels.

As it can be further seen from the examples, the inventors demonstrated, for instance in example 3, that the antigen binding proteins of the invention have an improved stability, in comparison to a reference protein, for instance, in comparison to an antigen binding protein comprising the CDRs of variant CDR6.

Accordingly, in one embodiment, the antigen binding proteins of the invention have an improved stability, optionally, in comparison to a reference protein. In context of the present invention, an improved stability refers for example to an increased physical stability when exposed to thermal stress. The newly developed antigen binding proteins of the invention can thus, better withstand stress conditions, especially thermal stress than the reference antigen binding protein.

The term “Stability” in context of the present invention refers to physical stability and can be evaluated qualitatively and/or quantitatively using various analytical techniques that are described in the art and are reviewed in for example Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). In context of the present invention, those methods refer in particular to the evaluation of aggregate formation (for example using size exclusion chromatography, by measuring turbidity, and/or by visual inspection. In order to measure stability a sample which comprises the antigen binding protein of the invention may be tested in a stability study, wherein a sample is exposed for a selected time period to a stress condition followed by quantitative and optionally qualitative analysis of the chemical and physical stability using an adequate analytical technique.

In one embodiment, the antigen binding proteins of the present invention are physical stable, for instance, when exposed to stress condition for a certain period of time, such as when exposed for, for instance, 14 days, to a temperature of 40° C.

“Physical Stability” refers substantially, in context of the present invention, to an antigen binding protein having no signs of aggregation, precipitation and/or denaturation.

Methods to access the physical stability are for example size exclusion chromatography (SEC), dynamic light scattering (DLS), light obscuration (LO) and colour and clarity determined by visual inspection.

“No Signs of Aggregation” means, for example, that a sample comprising the antigen binding protein, after having been exposed to a stress condition, such as, to a temperature of 40° C. for 14 days in a buffer, such as PBS, has a monomer content of more than 94%, more than 95%, more than 96%, more than 97%, more than 98%, more than 99%, such as a monomer content of 94% to 99%, 95% to 99%, 96% to 99%, 97% to 99%, typically, when measured by SEC, such as SEC-HPLC, in a buffer, such as PBS.

Accordingly, in one embodiment, the antigen binding proteins of the present invention have a reduced aggregation, for example, in comparison to a reference protein.

For size exclusion chromatography (SEC), a difference of 1%, 2%, 3%, 4%, preferably 1 or 2%, more preferably 1%, of the monomer content is considered as significantly different in the context of the invention under the tested conditions depending on the column used, operating pressure, and velocity of the buffer.

This means, when the reference antigen binding protein has a monomer content of 96% and the antigen binding protein of the invention has a monomer content of 97%, the monomer content of the antigen binding protein of the invention is significantly different and thus significantly increased in comparison to the reference antigen binding protein, when measured in the same conditions.

Furthermore, the inventors of the present invention demonstrated in the examples, in particular in examples 7 and 8, that the antigen binding proteins of the present invention binds the target antigen, i.e. PRAME peptide comprising the amino acid sequence SLLQHLIGL of SEQ ID NO: 8 in a complex with a MHC protein, preferably in complex with HLA-A*02, with high specificity.

The term “Specificity” generally denotes the capacity of an antigen binding protein to discriminate the target peptide sequence to which its binds (“epitope”) from closely related, highly homologous, peptide sequences.

In context of the present invention, the closely related, highly homologous, peptide sequences, also herein referred to as off-target peptides or similar peptides are the peptide IFIT1-001 consisting of the amino acid sequence (SEQ ID NO: 50), IFT17-003 consisting of the amino acid sequence (SEQ ID NO: 51), FADS2-001 consisting of the amino acid sequence (SEQ ID NO: 52) and CTBP1-001 consisting of the amino acid sequence (SEQ ID NO: 53).

The inventors performed in Example 7 experiments in order to identify residues of PRAME-004 that are relevant for binding by the antigen binding proteins of the invention and to further identify off targets. As a result, the inventors could identify amino acid positions 5 to 9 of SEQ ID NO: 8 to be relevant for binding.

Accordingly, in one embodiment the antigen binding protein binds to a PRAME variant of SEQ ID NO: 8 in a complex with a MHC protein, preferably in complex with HLA-A*02, with an affinity that is decreased in comparison to the affinity for the specific antigen, i.e. PRAME peptide comprising the amino acid sequence SLLQHLIGL of SEQ ID NO: 8 in a complex with a MHC protein, preferably in complex with HLA-A*02, and wherein the respective KD for the PRAME variant complex is increased by the factor of 4 and 10, preferably 4, and wherein a PRAME variant refers to a peptide of the amino acid sequence of SEQ ID NO: 8 wherein at least one of the amino acid positions 5, 6, 7, 8 or 9 of SEQ ID NO: 8 is substituted, preferably substituted into an alanine.

Furthermore, the inventors of the present invention demonstrated in the examples, in particular in example 7, that the antigen binding proteins of the present invention bind the target antigen, i.e. the PRAME-004 antigenic peptide in a complex with a MHC protein, preferably in complex with HLA-A*02, with high specificity, i.e. the antigen binding proteins specifically binds to the identified epitope.

Accordingly, the antigen binding proteins of the present invention specifically bind to a PRAME-004 antigenic peptide comprising or consisting of the amino acid sequence ‘SLLQHLIGL’ of SEQ ID NO: 8, wherein said antigenic peptide is in complex with a MHC protein, preferably in complex with HLA-A*02. In a preferred embodiment the antigen binding protein specifically binds to the structural epitope as defined herein of the PRAM E-004 antigenic peptide of SEQ ID NO: 8. In a more preferred embodiment the antigen binding protein specifically binds to the functional epitope as defined herein of the PRAME-004 antigenic peptide of SEQ ID NO: 8 More particularly, in one embodiment the antigen binding protein specifically binds to the functional epitope comprising or consisting of at least three or at least four or at least five amino acid positions of the PRAME-004 antigenic peptide of SEQ ID NO: 8, preferably, at least 3, at least 4, at least 5 amino acid positions, more preferably at least 3, such as 3 or 4, preferably 3, selected from the group consisting of the amino acid positions 5, 6, 7, 8 and 9, in particular 5, 7 and 9 of the amino acid sequence of SEQ ID NO: 8. The determination of the exact epitope might slightly vary depending on the method used and the cut-off values chosen.

“Does Not Significantly Bind” in the context of antigen binding proteins of the invention, in particular TCRs or fragments thereof or bispecific TCRs and fragments thereof, such as T cell-expressed antigen binding proteins, denotes, typically in a functional assay, for example, in a TCR activation assay, such as the IFN-gamma release described above, a response, such as signal that is detected for e.g. antigenic peptide variants, that is below 30%, below 25%, below 20%, below 15%, preferably below 30% of the response, i.e. the signal, obtained for the PRAME-004 peptide consisting of the amino acid sequence ‘SLLQHLIGL’ of SEQ ID NO: 8, preferably in the same experimental conditions. For example, in context of the present invention the antigen binding protein has a binding response for the at least one similar peptides/MHC complex that is less than 50%, less than 45%, less than 40%, less than 30%, less than 20%, less than 20%, less than 10%, less than 5%, less than 4%, less than 3%, less than 3% of the binding response of the same antigen binding protein to PRAME-004 antigenic peptide/MHC complex in the same experimental setting and at the same antigen binding protein concentration and/or, for example, in context of the present invention the antigen binding protein binds to the at least one similar peptide/MHC complex with an affinity that is decreased in comparison to the affinity for the specific antigen, i.e. the PRAME-004 antigenic peptide/MHC complex as described herein and wherein the respective KD for the respective similar peptide is increased by the factor of 5, 7, 10, 15, 20, 30, 40, 50, 100, preferably 20 to 100, more preferably 30 to 100, such as 40 to 100, typically 40 to 50. For example, when the antigen binding protein binds to the complex PRAME-004 /MHC with a K_{D} of 1 nM and the antigen binding protein binds to the complex of, for instance, IFIT1-001/MHC with a K_{D} of 100 nM then the antigen binding protein binds to IFIT1-001/MHC with a KD that is increased by a factor of 100 and thus, with an affinity that is decreased by a factor of 100. In these examples, the binding response, the dissociation constants and binding affinities are preferably measured using biolayer interferometry as described in example 4.

“Does Not Significantly Bind” in context of antigen binding proteins of the invention, in particular soluble antigen binding proteins of the invention, denotes, typically in a binding assay, for example biolayer interferometry, a K_{D} determined for an antigenic peptide variant that is increased by more than a factor of 3, more than 3.5, more than 4, more than 4.5, more than 5, preferably more than 3, such as 3 to 10, than the K_{D} determined for the for PRAME-004 peptide consisting of the amino acid sequence ‘SLLQHLIGL’ of SEQ ID NO: 8, preferably in the same experimental conditions, wherein the signal obtained for antigenic peptide variants is preferably a background signal, wherein the antigenic peptide variants or the PRAME-004 peptide are in complex with the MHC molecule.

According to the above, in one embodiment, the antigen binding protein of the invention, in particular a soluble antigen binding protein, binds to PRAME-004 antigenic peptide variant in a complex with a MHC protein, preferably in complex with HLA-A*02, with an affinity that is decreased in comparison to the affinity for the specific antigen, i.e. the PRAME-004 antigenic peptide as described in context of the invention in a complex with a MHC protein, preferably in complex with HLA-A*02, and wherein the respective K_{D} for PRAME-004 antigenic peptide variant complex is increased by more than a factor of 3, more than 3.5, more than 4, more than 4.5, more than 5, preferably more than 3, such as 3 to 10. The terms “affinity” and “K_{D}” are defined herein above in the section “Definitions”.

MHC proteins, in particular a MHC class I and HLA-A*02 proteins are as defined herein above in the section ‘Definitions’.

After having identified the positions in PRAM E-004 the inventors identified potential off target peptides (also referred to similar peptides in the following).

In particular, the peptides IFIT1-001, IFT17-003, FADS2-001 and CTBP1-001 are herein referred to as off-target peptides or similar peptides.

Accordingly, in a further related embodiment the antigen binding proteins of the invention, bind specifically to PRAM E-004 peptide or the PRAME epitope in a complex with a MHC protein, preferably in complex with HLA-A*02 and do not cross react with the off targets IFIT1-001, IFT17-003, FADS2-001 and CTBP1-001, preferably IFIT1-001, FADS2-001 and CTBP1-001, in a complex with a MHC protein, preferably in complex with HLA-A*02.

An antigen binding protein “Binds Specifically” to a first antigen when it is not significantly cross-reactive to a second antigen.

The antigen binding protein binding to antigen PRAME-004/MHC1 is “Not Significantly Cross-Reactive” to off target peptides/MHC when the affinities are very different for the two antigens. Affinity for off target peptides or similar peptides may not be measurable if the binding response is too low. In the present application antigen binding protein binding to PRAME-004/MHC1 is not significantly cross-reactive to off target peptides/MHC, when the binding response of the antigen binding protein to off targets/MHC is less than 5% of the binding response of the same antigen binding protein to PRAME-004/MHC1 in the same experimental setting and at the same antibody concentration.

The inventors demonstrated in the examples, that the antigen binding proteins of the present invention have the capacity to bind PRAME-004 with high affinity and to bind the peptides IFIT1-001, IFT17-003, FADS2-001 and CTBP1-001, in a complex with a MHC protein, preferably in complex with HLA-A*02, only with minimal or reduced binding affinity.

A “Minimal Affinity” herein refers to an affinity for the peptides IFIT1-001, I FT17-003, FADS2-001 and CTBP1-001, in a complex with a MHC protein, preferably in complex with HLA-A*02, preferably the peptides IFIT1-001, FADS2-001 and CTBP1-001 in a complex with a MHC protein, preferably in complex with HLA-A*02, that is decreased in comparison to the affinity for the specific antigen, i.e. PRAME peptide comprising the amino acid sequence SLLQHLIGL of SEQ ID NO: 8 in a complex with a MHC protein, preferably in complex with HLA-A*02, and wherein the respective K_{D}s for the off-target peptides or similar peptides is increased by the factor of 10, 100, 1000, preferably 100 or 1000, more preferably by 1000.

For example, when the antigen binding protein binds to the complex PRAME-004/MHC with a KD of 1 nM and the antigen binding protein binds to the complex of, for instance, FADS2-001 MHC with a KD of 100 nM then the antigen binding protein binds to FADS2-001/MHC with a KD that is increased by a factor of 100 and thus with an affinity that is decreased by a factor of 100.

This is an important advantage of the antigen binding proteins of the present invention since binding to off-target peptides or similar peptides might increase the risk of side effects, accordingly, the fact that the antigen binding proteins of the invention bind off target peptides only with low affinity makes it a promising anti-cancer treatment with regard to safety and efficacy.

In one embodiment, the antigen binding protein of the present invention do not significantly bind to the peptides IFIT1-001, IFT17-003, FADS2-001 and CTBP1-001, preferably IFIT1-001, FADS2-001 and CTBP1-001, more preferably IFIT1-001.

In one embodiment, the antigen binding protein of the present invention do not significantly bind to the peptide IFT17-003.

“Do Not Significantly Bind” herein is also used to refer to no significant detection of positive yeast cells when the peptides in a complex with a MHC protein, preferably in complex with HLA-A*02 are used to stain yeast cells displaying antigen binding protein on the surface as described herein in example 8 and when the positive yeast cells are, typically, measured via flow cytometric analysis. Methods to measure specificity are known to the skilled in the art and on typical example thereof is further described in the example section in Example 8.

The antigen binding proteins of the present invention specifically bind to the PRAME-004 antigenic peptide complex as specified herein above, more particularly to the epitope of SEQ ID NO: 8 as defined herein above and can distinguish between its respective target, i.e. the PRAME-004 antigenic peptide and similar peptides, to which it does not significantly bind.

The term “Specificity” or “Specifically Binds” denotes the capacity of an antigen binding protein to discriminate the target peptide sequence to which its binds (“epitope”) from similar epitopes, peptides or proteins, i.e. an antigen binding protein “Binds Specifically” to a first antigen when it is not significantly cross-reactive to a second.

“Similar Peptides” in context of the present invention may also be referred to as “Off-Targets” and relates to peptides comprising typically 8 to 11 amino acids in length. The similar peptides in context of the present invention are typically MHC presented. Furthermore, similar peptides in context of the present invention comprises or consists of an amino acid sequence that is similar to the amino acid sequence of the PRAME-004 antigenic peptide, more particular, peptides that, in comparison to the epitope of the PRAME-004 antigenic peptide, comprise an epitope wherein some or all amino acids are identical and/or similar in biochemical/biophysical characteristics of the amino acids, in comparison to the amino acids that constitute the epitope of the corresponding MAGE-A peptide. Due to this sequence similarity, similar peptides might be bound by an antigen binding protein, in this scenario, if, for example, the similar peptide is presented by a MHC protein and, thus bound by an antigen binding protein, for example a TCR, the ability of a given antigen binding protein, such as a TCR, to bind to a similar peptide will not lead to the desired T cell response but may lead to adverse reactions. Such adverse reactions may be “off-tumor” side effects, such as cross-reactivity of a specific TCR which cross-reacted with a peptide in healthy tissues as reported in Lowdell et al., Cytotherapy, published on Dec. 4, 2018, page 7. Similar peptides in context of the present invention were selected from a database of, for instance, normal tissue-presented HLA-A*02 bound peptides (XPRESIDENT database) using a similarity scoring within the binding-relevant positions 5-9 of PRAME-004 peptide. The antigen binding proteins of the present invention are thus engineered to avoid binding to similar peptides, in particular to the similar peptides listed herein below but maintain their affinity for the target peptide.

Accordingly, the similar peptides in context of the present invention are selected from Table 4.

In one embodiment, the antigen binding proteins of the invention, in particular TCRs or fragments thereof or bispecific TCRs and fragments thereof, such as T cell-expressed antigen binding proteins, do not bind or do not significantly bind at least 1, such as at least 2, at least 3, at least 4, at least 5, such as 1, 2, 3, 4, 5, preferably at least 3 or 3 or all of the similar peptides selected from the list consisting of IFIT1-001 (SEQ ID NO: 50), IFT17-003 (SEQ ID NO: 51), FADS2-001 (SEQ ID NO: 52) and CTBP1-001 (SEQ ID NO: 53), ATP1A1-001 (SEQ ID NO: 125), NADK-002 (SEQ ID NO: 126), ITSN1-001 (SEQ ID NO: 127), SMA-003 (SEQ ID NO: 28), VPS13A-001 (SEQ ID NO: 129), SF3B3-005 (SEQ ID NO: 130), NCAM2-001 (SEQ ID NO: 131), KNT-001 (SEQ ID NO: 132), EHD4-001 (SEQ ID NO: 133), EHD-001 (SEQ ID NO: 134), MCMB-002 (SEQ ID NO: 135), HSPAS-001 (SEQ ID NO: 136), CEBPZ-002 (SEQ ID NO: 137), DCAF12-001 (SEQ ID NO: 138), EHD2-002 (SEQ ID NO: 139), KIAA1324-001 (SEQ ID NO: 140), MYO1A-001 (SEQ ID NO: 141), SERPINA6-001 (SEQ ID NO: 142), SMARCD1-001 (SEQ ID NO: 143), TSC1-001 (SEQ ID NO: 144), UGT-005 (SEQ ID NO: 145), VAV1-001 (SEQ ID NO: 146), WDFY3-004 (SEQ ID NO: 147) when said similar peptide is in a complex with a MHC protein, preferably in complex with HLA-A*02. This is an important advantage in context of the present invention since binding to similar peptides, i.e. off-target peptides, may increase the risk of side effects, the fact that the antigen binding proteins of the invention do not cross-react with the herein listed similar peptides makes it a promising anti-cancer treatment with regard to safety and efficacy.

“Do Not Significantly Bind” may also be referred to as “do not cross-react” in context of similar peptides and in the context of the antigen binding proteins of the invention, in particular TCRs or fragments thereof or bispecific TCRs and fragments thereof, such as T cell-expressed antigen binding proteins and herein refers, for example, to a functional response that is measured in a functional assay for the antigen binding protein to a similar peptide/MHC in comparison to MAGE-A and wherein the response of the antigen binding protein to similar peptides/MHC is less than 30%, less than 20%, less than 10%, less than 5%, such as 8%, 6%, 5%, preferably 5% of the response of the same antigen binding protein to PRAME-004 antigenic peptide/MHC complex in the same experimental setting. In one example, the response is a functional response of T cells expressing said antigen binding protein and determined using an IFN-gamma release assay in context of the similar peptides.

Furthermore, in one embodiment, the antigen binding protein of the invention, in particular a soluble antigen binding protein of the invention, does not bind or does not significantly bind to at least one similar peptide, such as at least 2, at least 3, at least 4, at least 5, such as 1, 2, 3, 4, 5, preferably at least 3 or 3 or all similar peptides selected from the group of peptides consisting of IFIT1-001 (SEQ ID NO: 50), IFT17-003 (SEQ ID NO: 51), FADS2-001 (SEQ ID NO: 52) and CTBP1-001 (SEQ ID NO: 53) 001, when said similar peptide is in a complex with a MHC protein, preferably in complex with HLA-A*02.

The inventors of the present invention could demonstrate that the antigen binding proteins, in particular TCER® molecules show T cell-mediated cytotoxicity in a PRAME-positive tumor cell line by LDH release assay while human normal tissue cells were not affected by co-incubation with the TCER® molecule. This in vitro-experiments evidences the safety of the antigen binding proteins of the invention in an in vitro assessment and document that the cytotoxic effect is highly selective for the tumor cell line, i.e. for tumor tissue. The molecules of the inventions therefore, show beneficial safety profiles.

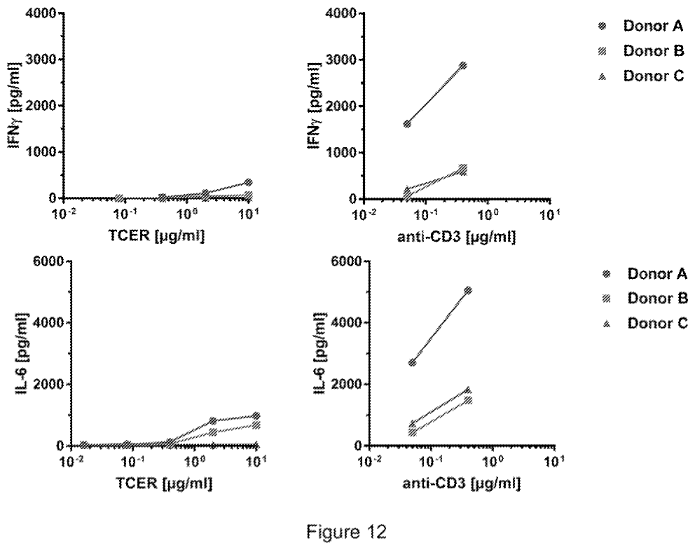

The inventors of the present invention could further demonstrate that the antigen binding proteins led to reduced cytokine release in an ex vivo model (FIG. 12). Whole blood samples incubated with an antigen binding protein of the invention shows significantly reduced IFN-γ and interleukin-6 (IL-6) levels compared to a control TCER® molecule and anti-CD3. These results support the findings that the antigen binding proteins of the invention show beneficial safety profiles.

The inventors of the present invention demonstrated that the CDRs that were initially obtained from TCR alpha and beta variable domains may be used in antigen binding proteins that have different formats than TCRs. For example, in the experimental section the inventors used the CDRs of the TCR alpha and beta variable domains in F_{c}-containing bispecific TCR/mAb diabody comprising a first polypeptide with Valpha-L_{Y}-V_{L}(CD3)-F_{c} and a second polypeptide with V_{H}(CD3)-Lₓ-Vbeta-F_{c}, wherein Valpha and Vbeta are alpha and beta variable domains comprising the CDRS as defined in context of the present invention, L_{Y} and Lₓ are linker, V_{L} (CD3) and V_{H}(CD3) are heavy and light chain variable domains of an anti-CD3 antibody, for instance, the mouse monoclonal anti-CD3 antibody UCHT1.

Furthermore, the inventors used the CDRs of the TCR alpha and beta variable domains in single chain TCR constructs, such as a bispecific TCR comprising a scTCR, in particular, comprising a first polypeptide with V_{H}(CD3)-C_{H1}-Valpha-L_{z}-Vbeta and a second polypeptide with V_{L}(CD3)-C_{L} wherein V_{H}(CD3), V_{L}(CD3), Valpha and Vbeta are as defined above and L_{z} is a linker and C_{H1} is the constant domain of the heavy chain and C_{L} is a constant region of the light chain.

Accordingly, the skilled in the art understands from these two experiments, as described in example 1, that indeed the CDRs as herein described may be used in different antigen binding proteins of the invention.

In one embodiment, the epitope and binding characteristics are conserved when the format of an antigen binding protein is changed (i.e. the CDRs of the first and second variable domain are the same).

In one embodiment, the antigen binding protein is an antibody or a fragment thereof or a T cell receptor (TCR) or fragment thereof.

The antibody and TCR are as defined herein above in the section ‘Definitions’.

In one embodiment, the antigen binding protein is bispecific.

Accordingly, the bispecific antigen binding protein is also herein referred to as “bispecific molecule”.

In one embodiment, the antigen binding protein is of human origin, which is understood as being generated from a human antigen locus and therefore comprising human sequences, in particular, human TCR or antibody sequences.

In one embodiment, the antigen binding protein of the invention is characterized as affinity maturated antigen binding protein, which is capable of specifically and selectively binding/recognizing the PRAME-004/MHC complex antigen.

More particularly, in one embodiment, the antigen binding protein is an antibody or a fragment thereof, such as a bispecific antibody or a fragment thereof, or a T cell receptor (TCR) or fragment thereof, or a bispecific T cell receptor or a fragment thereof.

The antibody, the bispecific antibody and T cell receptor (TCR) are as defined herein above in the section ‘Definitions’.

An antigen binding protein of the present invention preferably retains the antigen binding/recognizing ability of the parent molecule, in particular its specificity and/or selectivity as defined above, wherein the parent molecule may be either the parental TCR or, preferably, a bispecific antibody comprising the variable domains of said parental TCR. In one embodiment, such binding functionality may be retained by the presence of a first polypeptide and a second polypeptide comprising the CDRs as herein defined. In a further embodiment, such binding functionality may be retained by the presence of first polypeptide and a second polypeptide comprising the CDRs and FR domains as herein defined.

As already mentioned above, the binding functionality of the CDRs in context of the invention may be provided in the framework of an antibody. For example, CDR amino acid sequences as defined in context of the invention, possibly including additional 3, 2 or 1 N and/or C terminal framework residues, may be directly grafted into an antibody variable heavy/light chain sequence. More particularly, the CDRa1, CDRa2 and CDRa3 domains may be grafted in the variable heavy chain amino acid sequence and CDRb1, CDRb2 and CDRb3 may be grafted in the variable light chain amino acid sequence, or vice versa.

As it will also be understood, in some embodiments, in the framework of an antibody the variable light chain domain of an antibody may be replaced by the alpha variable domain as defined in context of the invention and the variable heavy chain domain may be replaced by the beta variable domain, or vice versa.

The inventors of the present invention furthermore discovered that specific mutations in the Framework region of the antigen binding proteins have an advantageous effect. In particular, the mutations R86S, V₉₁ I and T93N in the beta variable domain increase the stability of the protein, accordingly, the antigen binding proteins of the invention comprise framework regions with an amino acid sequence with the mutations R86S, V₉₁I and T93N or S85F, those mutations are indicated according to the IMGT nomenclature and they are located in the beta variable domain in FR3-b. Furthermore, those antigen binding proteins comprise the mutation F55S, said mutation is also indicated according to the IMGT nomenclature and is located in the alpha variable domain in FR2-a.

Accordingly, in one embodiment, the said first variable domain of the antigen binding protein of the invention, further comprises one or more framework regions selected from the group consisting of FR1-a, FR2-a, FR3-a and FR4-a, wherein
FR1-a comprises or consists of the amino acid sequence of QKEVEQNSGPLSVPEGAIASLNCTYS SEQ ID NO: 54 or an amino acid sequence at least 85% identical to SEQ ID NO: 54,
FR2-a comprises or consists of the amino acid sequence of FFWYRQYSGKSPELIMS SEQ ID NO: 55 or an amino acid sequence at least 85% identical to SEQ ID NO: 55,
FR3-a comprises or consists of the amino acid sequence of KEDGRFTAQLNKASQYVSLLIRDSQPSDSATYL SEQ ID NO: 56 or an amino acid sequence at least 85% identical to SEQ ID NO: 56,
FR4-a comprises or consists of the amino acid sequence of GTGTRLTIIPNIQN SEQ ID NO: 57 or an amino acid sequence at least 85% identical to SEQ ID NO: 57, and said second variable domain further comprises one or more framework regions selected from the group consisting of FR1-b, FR2-b, FR3-b and FR4-b, wherein
FR1-b comprises or consists of the amino acid sequence of KAGVTQTPRYLIKTRGQQVTLSCSPI SEQ ID NO: 58 or an amino acid sequence at least 85% identical to SEQ ID NO: 58,
FR2-b comprises or consists of the amino acid sequence of VSWYQQTPGQGLQFLFE SEQ ID NO: 59 or an amino acid sequence at least 85% identical to SEQ ID NO: 59,
FR3-b comprises or consists of the amino acid sequence of RNKGNFPGRFSGRQFSNSSSEMNISNLELGDSALYL SEQ ID NO: 60 or an amino acid sequence at least 85% identical to SEQ ID NO: 60, or
FR4-b comprises or consists of the amino acid sequence of GPGTRLTVTEDLKN SEQ ID NO: 62 or an amino acid sequence at least 85% identical to SEQ ID NO: 62.

Variants of the antigen binding proteins as described herein are contemplated and explicitly referred to using the wording “At Least 85% Identical to a Reference Sequence” as defined herein above in the section definitions. For instance, the sequence FR1-a, FR2-a, FR3-a and FR4-a and FR1-b, FR2-b, FR3-b and FR4-b may differ from the reference sequences SEQ ID NO:54, SEQ ID NO:55, SEQ ID NO:56, SEQ ID NO:57, SEQ ID NO:58, SEQ ID NO:59, SEQ ID NO:60, and SEQ ID NO:62 , as appropriate, by at least one amino acid substitution(s), in particular by at least one conservative amino acid substitution(s) and/or substitution(s) with canonical residues. In particular, the sequences FR1-a, FR2-a, FR3-a and FR4-a and FR1-b, FR2-b, FR3-b and FR4-b of the first and the second variable domain may differ from the reference sequences SEQ ID NO:54, SEQ ID NO:55, SEQ ID NO:56, SEQ ID NO:57, SEQ ID NO:58, SEQ ID NO:59, SEQ ID NO:60and SEQ ID NO:62 by conservative amino acid substitution(s), only.

Modifications and changes may be made in the amino acid sequence of the antigen binding protein of the present invention, and in the DNA sequences encoding them, and still result in a functional antigen binding protein or polypeptide with desirable characteristics.

Accordingly, in one embodiment, the present invention refers to an antigen binding protein comprising
(i) a first polypeptide chain comprising a first variable domain comprising or consisting of the amino acid sequence selected from the group consisting of SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99 and SEQ ID NO:100 or an amino acid sequence at least 85% identical to the amino acid sequence selected from the group consisting of SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, and
   a second polypeptide chain comprising a second variable domain comprising or consisting of the amino acid sequence of SEQ ID NO: 83 or an amino acid sequence at least 85% identical to SEQ ID NO: 83, or
(ii) a first polypeptide chain comprising a first variable domain comprising or consisting of the amino acid sequence selected from the group consisting of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 180, or an amino acid sequence at least 85% identical to the amino acid sequence selected from the group consisting of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 180, and
   a second polypeptide chain comprising a second variable domain comprising or consisting of the amino acid sequence of SEQ ID NO: 83 or an amino acid sequence at least 85% identical to SEQ ID NO: 83, or
(iii) a first polypeptide chain comprising a first variable domain comprising or consisting of the amino acid sequence selected from the group consisting of SEQ ID NO: 113, SEQ ID NO: 114, SEQ ID NO: 115, SEQ ID NO: 116, SEQ ID NO: 63, SEQ ID NO: 118 or an amino acid sequence at least 85% identical to the amino acid sequence selected from the group consisting of SEQ ID NO: 113, SEQ ID NO: 114, SEQ ID NO: 115, SEQ ID NO: 116, SEQ ID NO: 63, SEQ ID NO: 118, and
   a second polypeptide chain comprising a second variable domain comprising or consisting of the amino acid sequence of SEQ ID NO: 112 or an amino acid sequence at least 85% identical to SEQ ID NO: 112;
   preferably a first polypeptide and a second polypeptide as defined in ii) and iii).

Variants of the antigen binding proteins as described herein are contemplated and explicitly referred to using the wording “At Least 85% Identical to a Reference Sequence” as defined herein above in the section definitions. For instance, the sequence of the first variable domain may differ from the reference sequences as defined in i), ii) and iii), as appropriate, by at least one amino acid substitution(s), in particular by at least one conservative amino acid substitution(s) and/or substitution(s) with canonical residues. In particular, the sequences of the first and the second variable domain may differ from the reference sequences as defined in i), ii) and iii) by conservative amino acid substitution(s), only.

Modifications and changes may be made in the amino acid sequence of the antigen binding protein of the present invention, and in the corresponding DNA sequences, respectively, and still result in a functional antigen binding protein or polypeptide with desirable characteristics. Modifications may be made in the first or second variable domain, in particular in the framework regions or in the CDRs comprised in the first or second variable domain.

In one embodiment, the present invention refers to an antigen binding protein comprising
(i) a first polypeptide chain comprising a first variable domain comprising or consisting of the amino acid sequence selected from the group consisting of, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO:96, SEQ ID NO: 97, SEQ ID NO: 98, SEQ ID NO: 99 and SEQ ID NO: 100 or a variant thereof comprising or consisting of an amino acid sequence at least 85%, at least 90% or at least 95% identical to the amino acid sequence selected from the group consisting of SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, and
   wherein the CDRs of said first variable domain or variant preferably comprises the amino acid sequence of CDRa1 of SEQ ID NO: 11, CDRa2 of SEQ ID NO: 17 and CDRa3 of SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 32, SEQ ID NO: 33, SEQ ID NO: 34, SEQ ID NO: 35, SEQ ID NO: 36, SEQ ID NO: 38, SEQ ID NO: 181 or SEQ ID NO: 182, preferably comprising one or two amino acid substitutions, and

1a second polypeptide chain comprising a second variable domain comprising or consisting of the amino acid sequence of SEQ ID NO: 83 or a variant thereof comprising or consisting of an amino acid sequence at least 85%, at least 90% or at least 95% identical to SEQ ID NO: 83,
wherein the CDRs of said second variable domain or variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions, or
(ii) a first polypeptide chain comprising a first variable domain comprising or consisting of the amino acid sequence selected from the group consisting of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 180, SEQ ID NO: 110, SEQ ID NO: 111 or a variant thereof comprising or consisting of an amino acid sequence at least 85%, at least 90% or at least 95% identical to the amino acid sequence selected from the group consisting of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 180, SEQ ID NO: 110, SEQ ID NO: 111, and
   wherein the CDRs of said first variable domain or variant thereof preferably comprises the amino acid sequence of CDRa1 of SEQ ID NO: 16, CDRa2 of SEQ ID NO: 17 and CDRa3 of SEQ ID NO: 38, SEQ ID NO: 24, SEQ ID NO: 26, SEQ ID NO: 33, SEQ ID NO: 39, SEQ ID NO: 40, SEQ ID NO: 42, SEQ ID NO: 43, SEQ ID NO: 179, SEQ ID NO: 44, SEQ ID NO: 21, preferably comprising one or two amino acid substitutions, and
   a second polypeptide chain comprising a second variable domain comprising or consisting of the amino acid sequence of SEQ ID NO: 83 or a variant thereof comprising or consisting of an amino acid sequence at least 85%, at least 90% or at least 95% identical to SEQ ID NO: 83, and
   wherein the CDRs of said second variable domain or variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions, or
(iii) a first polypeptide chain comprising a first variable domain comprising or consisting of the amino acid sequence selected from the group consisting of SEQ ID NO: 113, SEQ ID NO: 114, SEQ ID NO: 115, SEQ ID NO: 116, SEQ ID NO: 63, SEQ ID NO: 118 or a variant thereof comprising or consisting of an amino acid sequence at least 85%, at least 90% or at least 95% identical to the amino acid sequence selected from the group consisting of SEQ ID NO: 113, SEQ ID NO: 114, SEQ ID NO: 115, SEQ ID NO: 63, SEQ ID NO: 118, and
   wherein the CDRs of said first variable domain or variant thereof preferably comprises the amino acid sequence of CDRa1 of SEQ ID NO: 16 or SEQ ID NO: 11, CDRa2 of SEQ ID NO: 17 and CDRa3 of SEQ ID NO: 40, SEQ ID NO: 38, SEQ ID NO: 39, SEQ ID NO: 26 or SEQ ID NO: 21, preferably comprising one or two amino acid substitutions, and
   a second polypeptide chain comprising a second variable domain comprising or consisting of the amino acid sequence of SEQ ID NO: 112 or a variant thereof comprising or consisting of an amino acid sequence at least 85%, at least 90% or at least 95% identical to SEQ ID NO: 112, and
   wherein the CDRs of said second variable domain or variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions.

In one embodiment, the present invention refers to an antigen binding protein comprising
(i) a first polypeptide chain comprising a first variable domain comprising or consisting of the amino acid sequence selected from the group consisting of SEQ ID NO: 85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99 and SEQ ID NO:100, or a variant thereof comprising or consisting of an amino acid sequence at least 85% identical, preferably at least 90% or 95% identical to the amino acid sequence selected from the group consisting of SEQ ID NO: SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, and wherein said variant preferably comprises the amino acid sequence of CDRa1 of SEQ ID NO: 11, CDRa2 of SEQ ID NO: 17 and CDRa3 of SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 32, SEQ ID NO: 33, SEQ ID NO: 34, SEQ ID NO: 35, SEQ ID NO: 36, SEQ ID NO: 38, SEQ ID NO: 181 or SEQ ID NO: 182 and
wherein the first variable domain preferably further comprises at IMGT position 55 serine, and
(ii) a second polypeptide chain comprising a second variable domain comprising or consisting of the amino acid sequence of SEQ ID NO: 83 or a variant thereof comprising or consisting of an amino acid sequence at least 85% identical, preferably at least 90% or 95% identical to SEQ ID NO: 83, and
wherein said variant preferably comprises the amino acid sequence of CDRb1 of SEQ ID NO: 45, CDRb2 of SEQ ID NO: 46 and CDRb3 of SEQ ID NO: 48 and
wherein the second variable domain preferably further comprises at IMGT position 85 phenylalanine, at IMGT position 86 serine, at IMGT position 91 isoleucine, at IMGT position 93 asparagine or combinations thereof.

Accordingly, in one embodiment, the antigen binding protein comprises a first polypeptide chain comprising a first variable domain comprising or consisting of the amino acid sequence of, SEQ ID NO: 85 or a variant thereof comprising or consisting of an amino acid sequence at least 85%, at least 90% or at least 95% identical to SEQ ID NO: 85, wherein the CDRs of said first variable domain or a variant thereof comprise CDRa1 of SEQ ID NO: 11, CDRa2 of SEQ ID NO: 17 and CDRa3 of SEQ ID NO: 23, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 85 or a variant thereof comprising or consisting of an amino acid sequence at least 85%, at least 90% or at least 95% identical to SEQ ID NO: 85, wherein the CDRs of said first variable domain or a variant thereof comprise CDRa1 of SEQ ID NO: 11, CDRa2 of SEQ ID NO: 17 and CDRa3 of SEQ ID NO: 23, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 85 or a variant thereof comprising or consisting of an amino acid sequence at least 85%, at least 90% or at least 95% identical to SEQ ID NO: 85, wherein the CDRs of said first variable domain or a variant thereof comprise CDRa1 of SEQ ID NO: 11, CDRa2 of SEQ ID NO: 17 and CDRa3 of SEQ ID NO: 23, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 85 or a variant thereof comprising or consisting of an amino acid sequence at least 85%, at least 90% or at least 95% identical to SEQ ID NO: 85, wherein the CDRs of said first variable domain or a variant thereof comprise CDRa1 of SEQ ID NO: 11, CDRa2 of SEQ ID NO: 17 and CDRa3 of SEQ ID NO: 23, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 85 or a variant thereof comprising or consisting of an amino acid sequence at least 85%, at least 90% or at least 95% identical to SEQ ID NO: 85, wherein the CDRs of said first variable domain or variant thereof comprise CDRa1 of SEQ ID NO: 11, CDRa2 of SEQ ID NO: 17 and CDRa3 of SEQ ID NO: 23, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 86 or a variant thereof comprising or consisting of an amino acid sequence at least 85%, at least 90% or at least 95% identical to SEQ ID NO: 86, wherein the CDRs of said first variable domain or a variant thereof comprise CDRa1 of SEQ ID NO: 11, CDRa2 of SEQ ID NO: 17 and CDRa3 of SEQ ID NO: 24, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 86 or a variant thereof comprising or consisting of an amino acid sequence at least 85%, at least 90% or at least 95% identical to SEQ ID NO: 86, wherein the CDRs of said first variable domain or a variant thereof comprise CDRa1 of SEQ ID NO: 11, CDRa2 of SEQ ID NO: 17 and CDRa3 of SEQ ID NO: 24, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 86 or a variant thereof comprising or consisting of an amino acid sequence at least 85%, at least 90% or at least 95% identical to SEQ ID NO: 86, wherein the CDRs of said first variable domain or a variant thereof comprise CDRa1 of SEQ ID NO: 11, CDRa2 of SEQ ID NO: 17 and CDRa3 of SEQ ID NO: 24, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 86 or a variant thereof comprising or consisting of an amino acid sequence at least 85%, at least 90% or at least 95% identical to SEQ ID NO: 86, wherein the CDRs of said first variable domain or a variant thereof comprise CDRa1 of SEQ ID NO: 11, CDRa2 of SEQ ID NO: 17 and CDRa3 of SEQ ID NO: 24, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions, wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 86 or a variant thereof comprising or consisting of an amino acid sequence at least 85%, at least 90% or at least 95% identical to SEQ ID NO: 86, wherein the CDRs of said first variable domain or a variant thereof comprise CDRa1 of SEQ ID NO: 11, CDRa2 of SEQ ID NO: 17 and CDRa3 of SEQ ID NO: 24, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID No: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 87 or a variant thereof comprising or consisting of an amino acid sequence at least 85%, at least 90% or at least 95% identical to SEQ ID NO: 87, wherein the CDRs of said first variable domain or a variant thereof comprise CDRa1 of SEQ ID NO: 11, CDRa2 of SEQ ID NO: 17 and CDRa3 of SEQ ID NO: 25, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 87 or a variant thereof comprising or consisting of an amino acid sequence at least 85%, at least 90% or at least 95% identical to SEQ ID NO: 87, wherein the CDRs of said first variable domain or a variant thereof comprise CDRa1 of SEQ ID NO: 11, CDRa2 of SEQ ID NO: 17 and CDRa3 of SEQ ID NO: 25, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 87 or a variant thereof comprising or consisting of an amino acid sequence at least 85%, at least 90% or at least 95% identical to SEQ ID NO: 87, wherein the CDRs of said first variable domain or a variant thereof comprise CDRa1 of SEQ ID NO: 11, CDRa2 of SEQ ID NO: 17 and CDRa3 of SEQ ID NO: 25, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 87 or a variant thereof comprising or consisting of an amino acid sequence at least 85%, at least 90% or at least 95% identical to SEQ ID NO: 87, wherein the CDRs of said first variable domain or a variant thereof comprise CDRa1 of SEQ ID NO: 11, CDRa2 of SEQ ID NO: 17 and CDRa3 of SEQ ID NO: 25, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 87 or a variant thereof comprising or consisting of an amino acid sequence at least 85%, at least 90% or at least 95% identical to SEQ ID NO: 87, wherein the CDRs of said first variable domain or a variant thereof comprise CDRa1 of SEQ ID NO: 11, CDRa2 of SEQ ID NO: 17 and CDRa3 of SEQ ID NO: 25, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 88 or a variant thereof comprising or consisting of an amino acid sequence at least 85%, at least 90% or at least 95% identical to SEQ ID NO: 88, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 88 or a variant thereof comprising or consisting of an amino acid sequence at least 85%, at least 90% or at least 95% identical to SEQ ID NO: 88, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 88 or a variant thereof comprising or consisting of an amino acid sequence at least 85%, at least 90% or at least 95% identical to SEQ ID NO: 88, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 88 or a variant thereof comprising or consisting of an amino acid sequence at least 85%, at least 90% or at least 95% identical to SEQ ID NO: 88, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 88 or a variant thereof comprising or consisting of an amino acid sequence at least 85%, at least 90% or at least 95% identical to SEQ ID NO: 88, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 89 or a variant thereof comprising or consisting of an amino acid sequence at least 85%, at least 90% or at least 95% identical to SEQ ID NO: 89, wherein the CDRs of said first variable domain or a variant thereof comprise CDRa1 of SEQ ID NO: 11, CDRa2 of SEQ ID NO: 17 and CDRa3 of SEQ ID NO: 27, preferably comprising one or two amino acid substitutions, and wherein
the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 89 or a variant thereof comprising or consisting of an amino acid sequence at least 85%, at least 90% or at least 95% identical to SEQ ID NO: 89, wherein the CDRs of said first variable domain or a variant thereof comprise CDRa1 of SEQ ID NO: 11, CDRa2 of SEQ ID NO: 17 and CDRa3 of SEQ ID NO: 27, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 89 or a variant thereof comprising or consisting of an amino acid sequence at least 85%, at least 90% or at least 95% identical to SEQ ID NO: 89, wherein the CDRs of said first variable domain or a variant thereof comprise CDRa1 of SEQ ID NO: 11, CDRa2 of SEQ ID NO: 17 and CDRa3 of SEQ ID NO: 27, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 89 or a variant thereof comprising or consisting of an amino acid sequence at least 85%, at least 90% or at least 95% identical to SEQ ID NO: 89, wherein the CDRs of said first variable domain or a variant thereof comprise CDRa1 of SEQ ID NO: 11, CDRa2 of SEQ ID NO: 17 and CDRa3 of SEQ ID NO: 27, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 89 or a variant thereof comprising or consisting of an amino acid sequence at least 85%, at least 90% or at least 95% identical to SEQ ID NO: 89, wherein the CDRs of said first variable domain or a variant thereof comprise CDRa1 of SEQ ID NO: 11, CDRa2 of SEQ ID NO: 17 and CDRa3 of SEQ ID NO: 27, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 90 or a variant thereof comprising or consisting of an amino acid sequence at least 85%, at least 90% or at least 95% identical to SEQ ID NO: 90, wherein the CDRs of said first variable domain or a variant thereof comprise CDRa1 of SEQ ID NO: 11, CDRa2 of SEQ ID NO: 17 and CDRa3 of SEQ ID NO: 28, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 90 or a variant thereof comprising or consisting of an amino acid sequence at least 85%, at least 90% or at least 95% identical to SEQ ID NO: 90, wherein the CDRs of said first variable domain or a variant thereof comprise CDRa1 of SEQ ID NO: 11, CDRa2 of SEQ ID NO: 17 and CDRa3 of SEQ ID NO: 28, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions, wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 90 or a variant thereof comprising or consisting of an amino acid sequence at least 85%, at least 90% or at least 95% identical to SEQ ID NO: 90, wherein the CDRs of said first variable domain or a variant thereof comprise CDRa 1 of SEQ ID NO: 11, CDRa2 of SEQ ID NO: 17 and CDRa3 of SEQ ID NO: 28, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine. In one embodiment, the antigen binding protein comprises a first polypeptide chain comprising a first variable domain comprising or consisting of the amino acid sequence of, SEQ ID NO: 90 or a variant thereof comprising or consisting of an amino acid sequence at least 85%, at least 90% or at least 95% identical to SEQ ID NO: 90, wherein the CDRs of said first variable domain or a variant thereof comprise CDRa1 of SEQ ID NO: 11, CDRa2 of SEQ ID NO: 17 and CDRa3 of SEQ ID NO: 28, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 90 or a variant thereof comprising or consisting of an amino acid sequence at least 85%, at least 90% or at least 95% identical to SEQ ID NO: 90, wherein the CDRs of said first variable domain or a variant thereof comprise CDRa1 of SEQ ID NO: 11, CDRa2 of SEQ ID NO: 17 and CDRa3 of SEQ ID NO: 28, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 91 or a variant thereof comprising or consisting of an amino acid sequence at least 85%, at least 90% or at least 95% identical to SEQ ID NO: 91, wherein the CDRs of said first variable domain or a variant thereof comprise CDRa1 of SEQ ID NO: 11, CDRa2 of SEQ ID NO: 17 and CDRa3 of SEQ ID NO: 29, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 91 or a variant thereof comprising or consisting of an amino acid sequence at least 85%, at least 90% or at least 95% identical to SEQ ID NO: 91, wherein the CDRs of said first variable domain or a variant thereof comprise CDRa1 of SEQ ID NO: 11, CDRa2 of SEQ ID NO: 17 and CDRa3 of SEQ ID NO: 29, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 91 or a variant thereof comprising or consisting of an amino acid sequence at least 85%, at least 90% or at least 95% identical to SEQ ID NO: 91, wherein the CDRs of said first variable domain or a variant thereof comprise CDRa1 of SEQ ID NO: 11, CDRa2 of SEQ ID NO: 17 and CDRa3 of SEQ ID NO: 29, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 91 or a variant thereof comprising or consisting of an amino acid sequence at least 85%, at least 90% or at least 95% identical to SEQ ID NO: 91, wherein the CDRs of said first variable domain or a variant thereof comprise CDRa1 of SEQ ID NO: 11, CDRa2 of SEQ ID NO: 17 and CDRa3 of SEQ ID NO: 29, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 91 or a variant thereof comprising or consisting of an amino acid sequence at least 85%, at least 90% or at least 95% identical to SEQ ID NO: 91, wherein the CDRs of said first variable domain or a variant thereof comprise CDRa1 of SEQ ID NO: 11, CDRa2 of SEQ ID NO: 17 and CDRa3 of SEQ ID NO: 29, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 92 or a variant thereof comprising or consisting of an amino acid sequence at least 85%, at least 90% or at least 95% identical to SEQ ID NO: 92, wherein the CDRs of said first variable domain or a variant thereof comprise CDRa1 of SEQ ID NO: 11, CDRa2 of SEQ ID NO: 17 and CDRa3 of SEQ ID NO: 30, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 92 or a variant thereof comprising or consisting of an amino acid sequence at least 85%, at least 90% or at least 95% identical to SEQ ID NO: 92, wherein the CDRs of said first variable domain or a variant thereof comprise CDRa1 of SEQ ID NO: 11, CDRa2 of SEQ ID NO: 17 and CDRa3 of SEQ ID NO: 30, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 92 or a variant thereof comprising or consisting of an amino acid sequence at least 85%, at least 90% or at least 95% identical to SEQ ID NO: 92, wherein the CDRs of said first variable domain or a variant thereof comprise CDRa1 of SEQ ID NO: 11, CDRa2 of SEQ ID NO: 17 and CDRa3 of SEQ ID NO: 30, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 92 or a variant thereof comprising or consisting of an amino acid sequence at least 85%, at least 90% or at least 95% identical to SEQ ID NO: 92, wherein the CDRs of said first variable domain or a variant thereof comprise CDRa1 of SEQ ID NO: 11, CDRa2 of SEQ ID NO: 17 and CDRa3 of SEQ ID NO: 30, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 92 or a variant thereof comprising or consisting of an amino acid sequence at least 85%, at least 90% or at least 95% identical to SEQ ID NO: 92, wherein the CDRs of said first variable domain or a variant thereof comprise CDRa1 of SEQ ID NO: 11, CDRa2 of SEQ ID NO: 17 and CDRa3 of SEQ ID NO: 30, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 93 or a variant thereof comprising or consisting of an amino acid sequence at least 85%, at least 90% or at least 95% identical to SEQ ID NO: 93, wherein the CDRs of said first variable domain or a variant thereof comprise CDRa1 of SEQ ID NO: 11, CDRa2 of SEQ ID NO: 17 and CDRa3 of SEQ ID NO: 181, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 93 or a variant thereof comprising or consisting of an amino acid sequence at least 85%, at least 90% or at least 95% identical to SEQ ID NO: 93, wherein the CDRs of said first variable domain or a variant thereof comprise CDRa1 of SEQ ID NO: 11, CDRa2 of SEQ ID NO: 17 and CDRa3 of SEQ ID NO: 181, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 93 or a variant thereof comprising or consisting of an amino acid sequence at least 85%, at least 90% or at least 95% identical to SEQ ID NO: 93, wherein the CDRs of said first variable domain or a variant thereof comprise CDRa1 of SEQ ID NO: 11, CDRa2 of SEQ ID NO: 17 and CDRa3 of SEQ ID NO: 181, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 93 or a variant thereof comprising or consisting of an amino acid sequence at least 85%, at least 90% or at least 95% identical to SEQ ID NO: 93, wherein the CDRs of said first variable domain or a variant thereof comprise CDRa1 of SEQ ID NO: 11, CDRa2 of SEQ ID NO: 17 and CDRa3 of SEQ ID NO: 181, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 93 or a variant thereof comprising or consisting of an amino acid sequence at least 85%, at least 90% or at least 95% identical to SEQ ID NO: 93, wherein the CDRs of said first variable domain or a variant thereof comprise CDRa1 of SEQ ID NO: 11, CDRa2 of SEQ ID NO: 17 and CDRa3 of SEQ ID NO: 181, preferably comprising one or two amino acid substitutions, and
wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 94 or a variant thereof comprising or consisting of an amino acid sequence at least 85%, at least 90% or at least 95% identical to SEQ ID NO: 94, wherein the CDRs of said first variable domain or a variant thereof comprise CDRa1 of SEQ ID NO: 11, CDRa2 of SEQ ID NO: 17 and CDRa3 of SEQ ID NO: 32, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 94 or a variant thereof comprising or consisting of an amino acid sequence at least 85%, at least 90% or at least 95% identical to SEQ ID NO: 94, wherein the CDRs of said first variable domain or a variant thereof comprise CDRa1 of SEQ ID NO: 11, CDRa2 of SEQ ID NO: 17 and CDRa3 of SEQ ID NO: 32, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 94 or a variant thereof comprising or consisting of an amino acid sequence at least 85%, at least 90% or at least 95% identical to SEQ ID NO: 94, wherein the CDRs of said first variable domain or a variant thereof comprise CDRa1 of SEQ ID NO: 11, CDRa2 of SEQ ID NO: 17 and CDRa3 of SEQ ID NO: 32, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 94 or a variant thereof comprising or consisting of an amino acid sequence at least 85%, at least 90% or at least 95% identical to SEQ ID NO: 94, wherein the CDRs of said first variable domain or a variant thereof comprise CDRa1 of SEQ ID NO: 11, CDRa2 of SEQ ID NO: 17 and CDRa3 of SEQ ID NO: 32, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 94 or a variant thereof comprising or consisting of an amino acid sequence at least 85%, at least 90% or at least 95% identical to SEQ ID NO: 94, wherein the CDRs of said first variable domain or a variant thereof comprise CDRa1 of SEQ ID NO: 11, CDRa2 of SEQ ID NO: 17 and CDRa3 of SEQ ID NO: 32, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 95 or a variant thereof comprising or consisting of an amino acid sequence at least 85%, at least 90% or at least 95% identical to SEQ ID NO: 95, wherein the CDRs of said first variable domain or a variant thereof comprise CDRa1 of SEQ ID NO: 11, CDRa2 of SEQ ID NO: 17 and CDRa3 of SEQ ID NO: 33, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 95 or a variant thereof comprising or consisting of an amino acid sequence at least 85%, at least 90% or at least 95% identical to SEQ ID NO: 95, wherein the CDRs of said first variable domain or a variant thereof comprise CDRa1 of SEQ ID NO: 11, CDRa2 of SEQ ID NO: 17 and CDRa3 of SEQ ID NO: 33, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 95 or a variant thereof comprising or consisting of an amino acid sequence at least 85%, at least 90% or at least 95% identical to SEQ ID NO: 95, wherein the CDRs of said first variable domain or a variant thereof comprise CDRa1 of SEQ ID NO: 11, CDRa2 of SEQ ID NO: 17 and CDRa3 of SEQ ID NO: 33, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 95 or a variant thereof comprising or consisting of an amino acid sequence at least 85%, at least 90% or at least 95% identical to SEQ ID NO: 95, wherein the CDRs of said first variable domain or a variant thereof comprise CDRa1 of SEQ ID NO: 11, CDRa2 of SEQ ID NO: 17 and CDRa3 of SEQ ID NO: 33, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 95 or a variant thereof comprising or consisting of an amino acid sequence at least 85%, at least 90% or at least 95% identical to SEQ ID NO: 95, wherein the CDRs of said first variable domain or a variant thereof comprise CDRa1 of SEQ ID NO: 11, CDRa2 of SEQ ID NO: 17 and CDRa3 of SEQ ID NO: 33, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 96 or a variant thereof comprising or consisting of an amino acid sequence at least 85%, at least 90% or at least 95% identical to SEQ ID NO: 96, wherein the CDRs of said first variable domain or a variant thereof comprise CDRa1 of SEQ ID NO: 11, CDRa2 of SEQ ID NO: 17 and CDRa3 of SEQ ID NO: 34, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 96 or a variant thereof comprising or consisting of an amino acid sequence at least 85%, at least 90% or at least 95% identical to SEQ ID NO: 96, wherein the CDRs of said first variable domain or a variant thereof comprise CDRa1 of SEQ ID NO: 11, CDRa2 of SEQ ID NO: 17 and CDRa3 of SEQ ID NO: 34, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 96 or a variant thereof comprising or consisting of an amino acid sequence at least 85%, at least 90% or at least 95% identical to SEQ ID NO: 96, wherein the CDRs of said first variable domain or a variant thereof comprise CDRa1 of SEQ ID NO: 11, CDRa2 of SEQ ID NO: 17 and CDRa3 of SEQ ID NO: 34, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 96 or a variant thereof comprising or consisting of an amino acid sequence at least 85%, at least 90% or at least 95% identical to SEQ ID NO: 96, wherein the CDRs of said first variable domain or a variant thereof comprise CDRa1 of SEQ ID NO: 11, CDRa2 of SEQ ID NO: 17 and CDRa3 of SEQ ID NO: 34, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 96 or a variant thereof comprising or consisting of an amino acid sequence at least 85%, at least 90% or at least 95% identical to SEQ ID NO: 96, wherein the CDRs of said first variable domain or a variant thereof comprise CDRa1 of SEQ ID NO: 11, CDRa2 of SEQ ID NO: 17 and CDRa3 of SEQ ID NO: 34, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 97 or a variant thereof comprising or consisting of an amino acid sequence at least 85%, at least 90% or at least 95% identical to SEQ ID NO: 97, wherein the CDRs of said first variable domain or a variant thereof comprise CDRa1 of SEQ ID NO: 11, CDRa2 of SEQ ID NO: 17 and CDRa3 of SEQ ID NO: 35, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 97 or a variant thereof comprising or consisting of an amino acid sequence at least 85%, at least 90% or at least 95% identical to SEQ ID NO: 97, wherein the CDRs of said first variable domain or a variant thereof comprise CDRa1 of SEQ ID NO: 11, CDRa2 of SEQ ID NO: 17 and CDRa3 of SEQ ID NO: 35, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 97 or a variant thereof comprising or consisting of an amino acid sequence at least 85%, at least 90% or at least 95% identical to SEQ ID NO: 97, wherein the CDRs of said first variable domain or a variant thereof comprise CDRa1 of SEQ ID NO: 11, CDRa2 of SEQ ID NO: 17 and CDRa3 of SEQ ID NO: 35, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 97 or a variant thereof comprising or consisting of an amino acid sequence at least 85%, at least 90% or at least 95% identical to SEQ ID NO: 97, wherein the CDRs of said first variable domain or a variant thereof comprise CDRa1 of SEQ ID NO: 11, CDRa2 of SEQ ID NO: 17 and CDRa3 of SEQ ID NO: 35, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 97 or a variant thereof comprising or consisting of an amino acid sequence at least 85%, at least 90% or at least 95% identical to SEQ ID NO: 97, wherein the CDRs of said first variable domain or a variant thereof comprise CDRa1 of SEQ ID NO: 11, CDRa2 of SEQ ID NO: 17 and CDRa3 of SEQ ID NO: 35, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 98 or a variant thereof comprising or consisting of an amino acid sequence at least 85%, at least 90% or at least 95% identical to SEQ ID NO: 98, wherein the CDRs of said first variable domain or a variant thereof comprise CDRa1 of SEQ ID NO: 11, CDRa2 of SEQ ID NO: 17 and CDRa3 of SEQ ID NO: 36, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 98 or a variant thereof comprising or consisting of an amino acid sequence at least 85%, at least 90% or at least 95% identical to SEQ ID NO: 98, wherein the CDRs of said first variable domain or a variant thereof comprise CDRa1 of SEQ ID NO: 11, CDRa2 of SEQ ID NO: 17 and CDRa3 of SEQ ID NO: 36, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 98 or a variant thereof comprising or consisting of an amino acid sequence at least 85%, at least 90% or at least 95% identical to SEQ ID NO: 98, wherein the CDRs of said first variable domain or a variant thereof comprise CDRa1 of SEQ ID NO: 11, CDRa2 of SEQ ID NO: 17 and CDRa3 of SEQ ID NO: 36, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 98 or a variant thereof comprising or consisting of an amino acid sequence at least 85%, at least 90% or at least 95% identical to SEQ ID NO: 98, wherein the CDRs of said first variable domain or a variant thereof comprise CDRa1 of SEQ ID NO: 11, CDRa2 of SEQ ID NO: 17 and CDRa3 of SEQ ID NO: 36, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 98 or a variant thereof comprising or consisting of an amino acid sequence at least 85%, at least 90% or at least 95% identical to SEQ ID NO: 98, wherein the CDRs of said first variable domain or a variant thereof comprise CDRa1 of SEQ ID NO: 11, CDRa2 of SEQ ID NO: 17 and CDRa3 of SEQ ID NO: 36, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ

ID NO: 99 or a variant thereof comprising or consisting of an amino acid sequence at least 85%, at least 90% or at least 95% identical to SEQ ID NO: 99, wherein the CDRs of said first variable domain or a variant thereof comprise CDRa1 of SEQ ID NO: 11, CDRa2 of SEQ ID NO: 17 and CDRa3 of SEQ ID NO: 182, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 99 or a variant thereof comprising or consisting of an amino acid sequence at least 85%, at least 90% or at least 95% identical to SEQ ID NO: 99, wherein the CDRs of said first variable domain or a variant thereof comprise CDRa1 of SEQ ID NO: 11, CDRa2 of SEQ ID NO: 17 and CDRa3 of SEQ ID NO: 182, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 99 or a variant thereof comprising or consisting of an amino acid sequence at least 85%, at least 90% or at least 95% identical to SEQ ID NO: 99, wherein the CDRs of said first variable domain or a variant thereof comprise CDRa1 of SEQ ID NO: 11, CDRa2 of SEQ ID NO: 17 and CDRa3 of SEQ ID NO:182, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 99 or a variant thereof comprising or consisting of an amino acid sequence at least 85%, at least 90% or at least 95% identical to SEQ ID NO: 99, wherein the CDRs of said first variable domain or a variant thereof comprise CDRa1 of SEQ ID NO: 11, CDRa2 of SEQ ID NO: 17 and CDRa3 of SEQ ID NO: 182, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 99 or a variant thereof comprising or consisting of an amino acid sequence at least 85%, at least 90% or at least 95% identical to SEQ ID NO: 99, wherein the CDRs of said first variable domain or a variant thereof comprise CDRa1 of SEQ ID NO: 11, CDRa2 of SEQ ID NO: 17 and CDRa3 of SEQ ID NO: 182, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 100 or a variant thereof comprising or consisting of an amino acid sequence at least 85%, at least 90% or at least 95% identical to SEQ ID NO: 100, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 100 or a variant thereof comprising or consisting of an amino acid sequence at least 85%, at least 90% or at least 95% identical to SEQ ID NO: 100, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 100 or a variant thereof comprising or consisting of an amino acid sequence at least 85%, at least 90% or at least 95% identical to SEQ ID NO: 100, wherein the CDRs of said first variable domain or a variant thereof comprise CDRa1 of SEQ ID NO: 11, CDRa2 of SEQ ID NO: 17 and CDRa3 of SEQ ID NO:38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 100 or a variant thereof comprising or consisting of an amino acid sequence at least 85%, at least 90% or at least 95% identical to SEQ ID NO: 100, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at position IMGT 93 asparagine.

In one embodiment, the antigen binding protein comprises a first polypeptide chain comprising a first variable domain comprising or consisting of the amino acid sequence of, SEQ ID NO: 100 or a variant thereof comprising or consisting of an amino acid sequence at least 85%, at least 90% or at least 95% identical to SEQ ID NO: 100, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In another embodiment, the present invention refers to an antigen binding protein comprising
(i) a first polypeptide chain comprising a first variable domain comprising or consisting of the amino acid sequence selected from the group consisting of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 180 or a variant thereof comprising or consisting of an amino acid sequence at least 85% identical, preferably at least 90% or 95% identical to the amino acid sequence selected from the group consisting of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 180, and
wherein said variant preferably comprises the amino acid sequence of CDRa1 of SEQ ID NO: 16, CDRa2 of SEQ ID NO: 17 and CDRa3 of SEQ ID NO: 38, SEQ ID NO: 24, SEQ ID NO: 26, SEQ ID NO: 33, SEQ ID NO: 39, SEQ ID NO: 40, SEQ ID NO: 42, SEQ ID NO: 43, SEQ ID NO: 179, SEQ ID NO: 44, SEQ ID NO: 21, and
wherein the first variable domain preferably further comprises at IMGT position 55 serine, and
(ii) a second polypeptide chain comprising a second variable domain comprising or consisting of the amino acid sequence of SEQ ID NO: 83 or a variant thereof comprising or consisting of an amino acid sequence at least 85% identical, preferably at least 90% or 95% identical to SEQ ID NO: 83, and
wherein said variant preferably comprises the amino acid sequence of CDRb1 of SEQ ID NO: 45, CDRb2 of SEQ ID NO: 46 and CDRb3 of SEQ ID NO: 48 and
wherein the second variable domain preferably further comprises at IMGT position 85 phenylalanine, at IMGT position 86 serine, at IMGT position 91 isoleucine, at IMGT position 93 asparagine or combinations thereof.

Accordingly, in one embodiment, the antigen binding protein comprises a first polypeptide chain comprising a first variable domain comprising or consisting of the amino acid sequence of, SEQ ID NO: 101 or a variant thereof comprising or consisting of an amino acid sequence at least 85%, at least 90% or at least 95% identical to SEQ ID NO: 101, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 101 or a variant thereof comprising or consisting of an amino acid sequence at least 85%, at least 90% or at least 95% identical to SEQ ID NO: 101, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 101 or a variant thereof comprising or consisting of an amino acid sequence at least 85%, at least 90% or at least 95% identical to SEQ ID NO: 101, wherein the CDRs of said first variable domain or a variant thereof comprise CDRa1 of SEQ ID NO: 16, CDRa2 of SEQ ID NO: 17 and CDRa3 of SEQ ID NO:38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 101 or a variant thereof comprising or consisting of an amino acid sequence at least 85%, at least 90% or at least 95% identical to SEQ ID NO: 101, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 101 or a variant thereof comprising or consisting of an amino acid sequence at least 85%, at least 90% or at least 95% identical to SEQ ID NO: 101, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 102 or a variant thereof comprising or consisting of an amino acid sequence at least 85%, at least 90% or at least 95% identical to SEQ ID NO: 102, wherein the CDRs of said first variable domain or a variant thereof comprise CDRa1 of SEQ ID NO: 16, CDRa2 of SEQ ID NO: 17 and CDRa3 of SEQ ID NO: 24, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 102 or a variant thereof comprising or consisting of an amino acid sequence at least 85%, at least 90% or at least 95% identical to SEQ ID NO: 102, wherein the CDRs of said first variable domain or a variant thereof comprise CDRa1 of SEQ ID NO: 16, CDRa2 of SEQ ID NO: 17 and CDRa3 of SEQ ID NO: 24, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 102 or a variant thereof comprising or consisting of an amino acid sequence at least 85%, at least 90% or at least 95% identical to SEQ ID NO: 102, wherein the CDRs of said first variable domain or a variant thereof comprise CDRa1 of SEQ ID NO: 16, CDRa2 of SEQ ID NO: 17 and CDRa3 of SEQ ID NO:24, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 102 or a variant thereof comprising or consisting of an amino acid sequence at least 85%, at least 90% or at least 95% identical to SEQ ID NO: 102, wherein the CDRs of said first variable domain or a variant thereof comprise CDRa1 of SEQ ID NO: 16, CDRa2 of SEQ ID NO: 17 and CDRa3 of SEQ ID NO: 24, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 102 or a variant thereof comprising or consisting of an amino acid sequence at least 85%, at least 90% or at least 95% identical to SEQ ID NO: 102, wherein the CDRs of said first variable domain or a variant thereof comprise CDRa1 of SEQ ID NO: 16, CDRa2 of SEQ ID NO: 17 and CDRa3 of SEQ ID NO: 24, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 103 or a variant thereof comprising or consisting of an amino acid sequence at least 85%, at least 90% or at least 95% identical to SEQ ID NO: 103, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID No: 48, preferably comprising one or two amino acid substititions.
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 103 or a variant thereof comprising or consisting of an amino acid sequence at least 85%, at least 90% or at least 95% identical to SEQ ID NO: 103, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 103 or a variant thereof comprising or consisting of an amino acid sequence at least 85%, at least 90% or at least 95% identical to SEQ ID NO: 103, wherein the CDRs of said first variable domain or a variant thereof comprise CDRa1 of SEQ ID NO: 16, CDRa2 of SEQ ID NO: 17 and CDRa3 of SEQ ID NO:26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 103 or a variant thereof comprising or consisting of an amino acid sequence at least 85%, at least 90% or at least 95% identical to SEQ ID NO: 103, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 103 or a variant thereof comprising or consisting of an amino acid sequence at least 85%, at least 90% or at least 95% identical to SEQ ID NO: 103, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at position 93 asparagine.

In one embodiment, the antigen binding protein comprises a first polypeptide chain comprising a first variable domain comprising or consisting of the amino acid sequence of, SEQ ID NO: 104 or a variant thereof comprising or consisting of an amino acid sequence at least 85%, at least 90% or at least 95% identical to SEQ ID NO: 104, wherein the CDRs of said first variable domain or a variant thereof comprise CDRa1 of SEQ ID NO: 16, CDRa2 of SEQ ID NO: 17 and CDRa3 of SEQ ID NO: 33, preferably comprising one or two amino acid substitutions, and wherein
the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 104 or a variant thereof comprising or consisting of an amino acid sequence at least 85%, at least 90% or at least 95% identical to SEQ ID NO: 104, wherein the CDRs of said first variable domain or a variant thereof comprise CDRa1 of SEQ ID NO: 16, CDRa2 of SEQ ID NO: 17 and CDRa3 of SEQ ID NO: 33, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 104 or a variant thereof comprising or consisting of an amino acid sequence at least 85%, at least 90% or at least 95% identical to SEQ ID NO: 104, wherein the CDRs of said first variable domain or a variant thereof comprise CDRa1 of SEQ ID NO: 16, CDRa2 of SEQ ID NO: 17 and CDRa3 of SEQ ID NO: 33, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 104 or a variant thereof comprising or consisting of an amino acid sequence at least 85%, at least 90% or at least 95% identical to SEQ ID NO: 104, wherein the CDRs of said first variable domain or a variant thereof comprise CDRa1 of SEQ ID NO: 16, CDRa2 of SEQ ID NO: 17 and CDRa3 of SEQ ID NO: 33, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 104 or a variant thereof comprising or consisting of an amino acid sequence at least 85%, at least 90% or at least 95% identical to SEQ ID NO: 104, wherein the CDRs of said first variable domain or a variant thereof comprise CDRa1 of SEQ ID NO: 16, CDRa2 of SEQ ID NO: 17 and CDRa3 of SEQ ID NO: 33, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 105 or a variant thereof comprising or consisting of an amino acid sequence at least 85%, at least 90% or at least 95% identical to SEQ ID NO: 105, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 105 or a variant thereof comprising or consisting of an amino acid sequence at least 85%, at least 90% or at least 95% identical to SEQ ID NO: 105, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 105 or a variant thereof comprising or consisting of an amino acid sequence at least 85%, at least 90% or at least 95% identical to SEQ ID NO: 105, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 105 or a variant thereof comprising or consisting of an amino acid sequence at least 85%, at least 90% or at least 95% identical to SEQ ID NO: 105, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID No: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 105 or a variant thereof comprising or consisting of an amino acid sequence at least 85%, at least 90% or at least 95% identical to SEQ ID NO: 105, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 106 or a variant thereof comprising or consisting of an amino acid sequence at least 85%, at least 90% or at least 95% identical to SEQ ID NO: 106, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 106 or a variant thereof comprising or consisting of an amino acid sequence at least 85%, at least 90% or at least 95% identical to SEQ ID NO: 106, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 106 or a variant thereof comprising or consisting of an amino acid sequence at least 85%, at least 90% or at least 95% identical to SEQ ID NO: 106, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 106 or a variant thereof comprising or consisting of an amino acid sequence at least 85%, at least 90% or at least 95% identical to SEQ ID NO: 106, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 106 or a variant thereof comprising or consisting of an amino acid sequence at least 85%, at least 90% or at least 95% identical to SEQ ID NO: 106, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a
first variable domain comprising or consisting of the amino acid sequence of, SEQ ID NO: 107 or a variant thereof comprising or consisting of an amino acid sequence at least 85%, at least 90% or at least 95% identical to SEQ ID NO: 107, wherein the CDRs of said first variable domain or a variant thereof comprise CDRa1 of SEQ ID NO: 16, CDRa2 of SEQ ID NO: 17 and CDRa3 of SEQ ID NO: 42, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 107 or a variant thereof comprising or consisting of an amino acid sequence at least 85%, at least 90% or at least 95% identical to SEQ ID NO: 107, wherein the CDRs of said first variable domain or a variant thereof comprise CDRa1 of SEQ ID NO: 16, CDRa2 of SEQ ID NO: 17 and CDRa3 of SEQ ID NO: 42, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 107 or a variant thereof comprising or consisting of an amino acid sequence at least 85%, at least 90% or at least 95% identical to SEQ ID NO: 107, wherein the CDRs of said first variable domain or a variant thereof comprise CDRa1 of SEQ ID NO: 16, CDRa2 of SEQ ID NO: 17 and CDRa3 of SEQ ID NO: 42, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 107 or a variant thereof comprising or consisting of an amino acid sequence at least 85%, at least 90% or at least 95% identical to SEQ ID NO: 107, wherein the CDRs of said first variable domain or a variant thereof comprise CDRa1 of SEQ ID NO: 16, CDRa2 of SEQ ID NO: 17 and CDRa3 of SEQ ID NO: 42, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 107 or a variant thereof comprising or consisting of an amino acid sequence at least 85%, at least 90% or at least 95% identical to SEQ ID NO: 107, wherein the CDRs of said first variable domain or a variant thereof comprise CDRa1 of SEQ ID NO: 16, CDRa2 of SEQ ID NO: 17 and CDRa3 of SEQ ID NO: 42, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 108 or a variant thereof comprising or consisting of an amino acid sequence at least 85%, at least 90% or at least 95% identical to SEQ ID NO: 108, wherein the CDRs of said first variable domain or a variant thereof comprise CDRa1 of SEQ ID NO: 16, CDRa2 of SEQ ID NO: 17 and CDRa3 of SEQ ID NO: 43, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 108 or a variant thereof comprising or consisting of an amino acid sequence at least 85%, at least 90% or at least 95% identical to SEQ ID NO: 108, wherein the CDRs of said first variable domain or a variant thereof comprise CDRa1 of SEQ ID NO: 16, CDRa2 of SEQ ID NO: 17 and CDRa3 of SEQ ID NO: 43, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 108 or a variant thereof comprising or consisting of an amino acid sequence at least 85%, at least 90% or at least 95% identical to SEQ ID NO: 108, wherein the CDRs of said first variable domain or a variant thereof comprise CDRa1 of SEQ ID NO: 16, CDRa2 of SEQ ID NO: 17 and CDRa3 of SEQ ID NO: 43, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 108 or a variant thereof comprising or consisting of an amino acid sequence at least 85%, at least 90% or at least 95% identical to SEQ ID NO: 108, wherein the CDRs of said first variable domain or a variant thereof comprise CDRa1 of SEQ ID NO: 16, CDRa2 of SEQ ID NO: 17 and CDRa3 of SEQ ID NO: 43, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 108 or a variant thereof comprising or consisting of an amino acid sequence at least 85%, at least 90% or at least 95% identical to SEQ ID NO: 108, wherein the CDRs of said first variable domain or a variant thereof comprise CDRa1 of SEQ ID NO: 16, CDRa2 of SEQ ID NO: 17 and CDRa3 of SEQ ID NO: 43, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 109 or a variant thereof comprising or consisting of an amino acid sequence at least 85%, at least 90% or at least 95% identical to SEQ ID NO: 109, wherein the CDRs of said first variable domain or a variant thereof comprise CDRa1 of SEQ ID NO: 16, CDRa2 of SEQ ID NO: 17 and CDRa3 of SEQ ID NO: 179, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 109 or a variant thereof comprising or consisting of an amino acid sequence at least 85%, at least 90% or at least 95% identical to SEQ ID NO: 109, wherein the CDRs of said first variable domain or a variant thereof comprise CDRa1 of SEQ ID NO: 16, CDRa2 of SEQ ID NO: 17 and CDRa3 of SEQ ID NO:179, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 109 or a variant thereof comprising or consisting of an amino acid sequence at least 85%, at least 90% or at least 95% identical to SEQ ID NO: 109, wherein the CDRs of said first variable domain or a variant thereof comprise CDRa1 of SEQ ID NO: 16, CDRa2 of SEQ ID NO: 17 and CDRa3 of SEQ ID NO: 179, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 109 or a variant thereof comprising or consisting of an amino acid sequence at least 85%, at least 90% or at least 95% identical to SEQ ID NO: 109, wherein the CDRs of said first variable domain or a variant thereof comprise CDRa1 of SEQ ID NO: 16, CDRa2 of SEQ ID NO: 17 and CDRa3 of SEQ ID NO: 179, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 109 or a variant thereof comprising or consisting of an amino acid sequence at least 85%, at least 90% or at least 95% identical to SEQ ID NO: 109, wherein the CDRs of said first variable domain or a variant thereof comprise CDRa1 of SEQ ID NO: 16, CDRa2 of SEQ ID NO: 17 and CDRa3 of SEQ ID NO: 179, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 180 or a variant thereof comprising or consisting of an amino acid sequence at least 85%, at least 90% or at least 95% identical to SEQ ID NO: 180, wherein the CDRs of said first variable domain or a variant thereof comprise CDRa1 of SEQ ID NO: 16, CDRa2 of SEQ ID NO: 17 and CDRa3 of SEQ ID NO: 179, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 180 or a variant thereof comprising or consisting of an amino acid sequence at least 85%, at least 90% or at least 95% identical to SEQ ID NO: 180, wherein the CDRs of said first variable domain or a variant thereof comprise CDRa1 of SEQ ID NO: 16, CDRa2 of SEQ ID NO: 17 and CDRa3 of SEQ ID NO: 179, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 180 or a variant thereof comprising or consisting of an amino acid sequence at least 85%, at least 90% or at least 95% identical to SEQ ID NO: 180, wherein the CDRs of said first variable domain or a variant thereof comprise CDRa1 of SEQ ID NO: 16, CDRa2 of SEQ ID NO: 17 and CDRa3 of SEQ ID NO: 179, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 190 or a variant thereof comprising or consisting of an amino acid sequence at least 85%, at least 90% or at least 95% identical to SEQ ID NO: 190, wherein the CDRs of said first variable domain or a variant thereof comprise CDRa1 of SEQ ID NO: 16, CDRa2 of SEQ ID NO: 17 and CDRa3 of SEQ ID NO: 179, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 180 or a variant thereof comprising or consisting of an amino acid sequence at least 85%, at least 90% or at least 95% identical to SEQ ID NO: 180, wherein the CDRs of said first variable domain or a variant thereof comprise CDRa1 of SEQ ID NO: 16, CDRa2 of SEQ ID NO: 17 and CDRa3 of SEQ ID NO: 179, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 110 or a variant thereof comprising or consisting of an amino acid sequence at least 85%, at least 90% or at least 95% identical to SEQ ID NO: 110, wherein the CDRs of said first variable domain or a variant thereof comprise CDRa1 of SEQ ID NO: 16, CDRa2 of SEQ ID NO: 17 and CDRa3 of SEQ ID NO: 44, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 110 or a variant thereof comprising or consisting of an amino acid sequence at least 85%, at least 90% or at least 95% identical to SEQ ID NO: 110, wherein the CDRs of said first variable domain or a variant thereof comprise CDRa1 of SEQ ID NO: 16, CDRa2 of SEQ ID NO: 17 and CDRa3 of SEQ ID NO: 44, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 110 or a variant thereof comprising or consisting of an amino acid sequence at least 85%, at least 90% or at least 95% identical to SEQ ID NO: 110, wherein the CDRs of said first variable domain or a variant thereof comprise CDRa1 of SEQ ID NO: 16, CDRa2 of SEQ ID NO: 17 and CDRa3 of SEQ ID NO: 44, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 110 or a variant thereof comprising or consisting of an amino acid sequence at least 85%, at least 90% or at least 95% identical to SEQ ID NO: 110, wherein the CDRs of said first variable domain or a variant thereof comprise CDRa1 of SEQ ID NO: 16, CDRa2 of SEQ ID NO: 17 and CDRa3 of SEQ ID NO: 44, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 110 or a variant thereof comprising or consisting of an amino acid sequence at least 85%, at least 90% or at least 95% identical to SEQ ID NO: 110, wherein the CDRs of said first variable domain or a variant thereof comprise CDRa1 of SEQ ID NO: 16, CDRa2 of SEQ ID NO: 17 and CDRa3 of SEQ ID NO: 44, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequences at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 111 or a variant thereof comprising or consisting of an amino acid sequence at least 85%, at least 90% or at least 95% identical to SEQ ID NO: 111, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 111 or a variant thereof comprising or consisting of an amino acid sequence at least 85%, at least 90% or at least 95% identical to SEQ ID NO: 111, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 111 or a variant thereof comprising or consisting of an amino acid sequence at least 85%, at least 90% or at least 95% identical to SEQ ID NO: 111, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 111 or a variant thereof comprising or consisting of an amino acid sequence at least 85%, at least 90% or at least 95% identical to SEQ ID NO: 111, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 111 or a variant thereof comprising or consisting of an amino acid sequence at least 85%, at least 90% or at least 95% identical to SEQ ID NO: 111, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In another embodiment, the present invention refers to an antigen binding protein comprising
(i) a first polypeptide chain comprising a first variable domain comprising or consisting of the amino acid sequence selected from the group consisting of SEQ ID NO: 113, SEQ ID NO: 114, SEQ ID NO: 115, SEQ ID NO: 116, SEQ ID NO: 63, SEQ ID NO: 118 or a variant thereof comprising or consisting of an amino acid sequence at least 85% identical, preferably at least 90% or 95% identical to the amino acid sequence selected from the group consisting of SEQ ID NO: 113, SEQ ID NO: 114, SEQ ID NO: 115, SEQ ID NO: 116, SEQ ID NO: 63, SEQ ID NO: 118, and
wherein said variant preferably comprises the amino acid sequence of CDRa1 of SEQ ID NO: 16 or SEQ ID NO: 11, CDRa2 of SEQ ID NO: 17 and CDRa3 of SEQ ID NO: 40, SEQ ID NO: 38, SEQ ID NO: 39, SEQ ID NO: 26 or SEQ ID NO: 21, and
wherein the first variable domain preferably further comprises at IMGT position 55 serine, and
(ii) a second polypeptide chain comprising a second variable domain comprising or consisting of the amino acid sequence of SEQ ID NO: 112 or a variant comprising or consisting of an amino acid sequence at least 85% identical, preferably at least 90% or 95% identical to SEQ ID NO: 112, and
wherein said variant preferably comprises the amino acid sequence of CDRb1 of SEQ ID NO: 45, CDRb2 of SEQ ID NO: 46 and CDRb3 of SEQ ID NO: 48 and
wherein the second variable domain preferably further comprises at IMGT position 85 phenylalanine, at IMGT position 86 serine, at IMGT position 91 isoleucine, at IMGT position 93 asparagine or combinations thereof.

### CDRa1 According to SEQ ID NO: 16

Accordingly, in one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein
the first variable domain preferably further comprises at IMGT position 55 serine; and a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein
the first variable domain preferably further comprises at IMGT position 55 serine; and a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

CDRa1 According to SEQ ID NO: 11

Accordingly, in one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 11, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 11, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 11, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 11, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 11, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 11, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 11, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 11, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 11, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 11, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 112 or a variant thereof comprising or consisting of an amino acid sequence at least 85%, at least 90% or at least 95% identical to SEQ ID NO: 112, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In another embodiment, the present invention refers to an antigen binding protein comprising
(i) a first polypeptide chain comprising a first variable domain comprising or consisting of the amino acid sequence selected from the group consisting of SEQ ID NO: 113, SEQ ID NO: 114, SEQ ID NO: 115, SEQ ID NO: 116, SEQ ID NO: 63, SEQ ID NO: 118 or a variant thereof comprising or consisting of an amino acid sequence at least 85% identical, preferably at least 90% or 95% identical to the amino acid sequence selected from the group consisting of SEQ ID NO: 113, SEQ ID NO: 114, SEQ ID NO: 115, SEQ ID NO: 116, SEQ ID NO: 63, SEQ ID NO: 118, and
wherein said variant preferably comprises the amino acid sequence of CDRa1 of SEQ ID NO: 16 or SEQ ID NO: 11, CDRa2 of SEQ ID NO: 17 and CDRa3 of SEQ ID NO: 40, SEQ ID NO: 38, SEQ ID NO: 39, SEQ ID NO: 26 or SEQ ID NO: 21, and
wherein the first variable domain preferably further comprises at IMGT position 55 serine, and
(ii) a second polypeptide chain comprising a second variable domain comprising or consisting of the amino acid sequence of SEQ ID NO: 83 or variant thereof comprising or consisting of an amino acid sequence at least 85% identical, preferably at least 90% or 95% identical to SEQ ID NO: 83,
wherein said variant preferably comprises the amino acid sequence of CDRb1 of SEQ ID NO: 45, CDRb2 of SEQ ID NO: 46 and CDRb3 of SEQ ID NO: 48 and
wherein the second variable domain preferably further comprises at IMGT position 85 phenylalanine, at IMGT position 86 serine, at IMGT position 91 isoleucine, at IMGT position 93 asparagine or combinations thereof.

### CDRa1 According to SEQ ID NO: 16

Accordingly, in one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 183 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 16, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 16, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 16, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 16, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 16, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

### CDRa1 According to SEQ ID NO: 11

Accordingly, in one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 11, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 11, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 11, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 11, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 113 or a variant thereof comprising or consisting of an amino acid sequence at least 85%, at least 90% or at least 95% identical to SEQ ID NO: 113, wherein the CDRs of said first variable domain or a variant thereof comprise CDRa1 of SEQ ID NO: 11, CDRa2 of SEQ ID NO: 17 and CDRa3 of SEQ ID NO: 40, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 114 or a variant thereof comprising or consisting of an amino acid sequence at least 85%, at least 90% or at least 95% identical to SEQ ID NO: 114, wherein the CDRs of said first variable domain or a variant thereof comprise CDRa1 of SEQ ID NO: 11, CDRa2 of SEQ ID NO: 17 and CDRa3 of SEQ ID NO: 38, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 11, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 11, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 11, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 11, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 115 or a variant thereof comprising or consisting of an amino acid sequence at least 85%, at least 90% or at least 95% identical to SEQ ID NO: 115, wherein the CDRs of said first variable domain or a variant thereof comprise CDRa1 of SEQ ID NO: 11, CDRa2 of SEQ ID NO: 17 and CDRa3 of SEQ ID NO: 39, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 116 or a variant thereof comprising or consisting of an amino acid sequence at least 85%, at least 90% or at least 95% identical to SEQ ID NO: 116, wherein the CDRs of said first variable domain or a variant thereof comprise CDRa1 of SEQ ID NO: 11, CDRa2 of SEQ ID NO: 17 and CDRa3 of SEQ ID NO: 26, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 63 or a variant thereof comprising or consisting of an amino acid sequence at least 85%, at least 90% or at least 95% identical to SEQ ID NO: 63, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 85 phenylalanine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 86 serine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 91 isoleucine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof preferably comprises the amino acid sequence of CDRb1 of SEQ ID NO: 45, CDRb2 of SEQ ID NO: 46 and CDRb3 of SEQ ID NO: 48, preferably comprising one or two amino acid substitutions,
wherein the second variable domain preferably further comprises at IMGT position 93 asparagine.

In one embodiment, the antigen binding protein comprises a first polypeptide chain comprising a first variable domain comprising or consisting of the amino acid sequence of, SEQ ID NO: 118 or a variant thereof comprising or consisting of an amino acid sequence at least 85%, at least 90% or at least 95% identical to SEQ ID NO: 118, wherein the CDRs of said first variable domain or a variant thereof comprise CDRa1 of SEQ ID NO: 11, CDRa2 of SEQ ID NO: 17 and CDRa3 of SEQ ID NO: 21, preferably comprising one or two amino acid substitutions, and wherein the first variable domain preferably further comprises at IMGT position 55 serine; and
a second polypeptide chain comprising a second variable domain comprising or consisting of amino acid sequence of SEQ ID NO: 83 or a variant thereof comprising or consisting of an amino acid sequence at least 85%, at least 90% or at least 95% identical to SEQ ID NO: 83, wherein the CDRs of said second variable domain or a variant thereof thereof preferably comprises the SEQ ID NO: 48, preferably comprising one or two amino acid substitutions,
wherein the second variable domain preferably further comprises at IMGT position 85 phenylalanine, at IMGT position 86 serine, at IMGT position 91 isoleucine and at IMGT position 93 asparagine.

In one embodiment, the first polypeptide and the second polypeptide are linked together, in particular via a covalent link.

A “Covalent Link” herein refers for example to a disulfide bridge or a peptide link or a covalent link via a linker, such as a polypeptide linker.

A “linker” or “linker sequence” or “polypeptide linker” is as defined herein above in the section definitions under “linker”.

In one embodiment the antigen binding protein of the invention further comprises one or more of the following:

(i) one or more further antigen binding sites;

(ii) a transmembrane region, optionally including a cytoplasmic signalling region;

(iii) a diagnostic agent;

(iv) a therapeutic agent; or

(v) PK modifying moiety.

As it is known to the skilled in the art an antigen binding site, for example, in context of antibodies, is typically formed by 6 CDRs that bind to an antigen.

In context of the present invention, the one or more binding site is preferably selected from a binding site that binds to an antigen, wherein said antigen is selected from the group consisting of the antigens CD3 (such as the CD3γ, CD3δ, and CD3ε chains), CD4, CD7, CD8, CD10, CD11b, CD11c, CD14, CD16, CD18, CD22, CD25, CD28, CD32a, CD32b, CD33, CD41, CD41b, CD42a, CD42b, CD44, CD45RA, CD49, CD55, CD56, CD61, CD64, CD68, CD94, CD90, CD117, CD123, CD125, CD134, CD137, CD152, CD163, CD193, CD203c, CD235a, CD278, CD279, CD287, Nkp46, NKG2D, GITR, FcεRI, TCRα/β and TCRγ/δ, HLA-DR or to an antigen of an effector cell, preferably CD3 or 28, more preferably CD3.

“CD3” is a protein complex and is composed of four distinct chains. In mammals, the complex contains a CD3γ chain, a CD3δ chain, and two CD3ε chains. These chains associate with a molecule known as the T cell receptor (TCR) and the ζ-chain to generate an activation signal in T lymphocytes.

“CD28” is also expressed on T cells and can provide co-stimulatory signals, which are required for T cell activation. CD28 plays important roles in T-cell proliferation and survival, cytokine production, and T-helper type-2 development.

In some embodiments, one of the binding sites of the antigen binding protein of the invention is able to bind a T-cell specific receptor molecule and/or a natural killer cell (NK cell) specific receptor molecule. In a particular embodiment, the antigen binding protein forms a complex with the CD3 T-Cell co-receptor.

In some embodiments a T-cell specific receptor is the CD3 T-Cell co-receptor.

In some embodiments, the T-cell specific receptor is CD28, CD134, 4-1 BB, CD5 or CD95.

“CD28” is also expressed on T cells and can provide co-stimulatory signals, which are required for T cell activation. CD28 plays important roles in T-cell proliferation and survival, cytokine production, and T-helper type-2 development.

“CD134” is also termed Ox40. CD134/OX40 is being expressed after 24 to 72 hours following activation and can be taken to define a secondary costimulatory molecule.

“4-1 BB” is capable of binding to 4-1 BB-Ligand on antigen presenting cells (APCs), whereby a costimulatory signal for the T cell is generated.

“CD5” is another example of a receptor predominantly found on T-cells, CD5 is also found on B cells at low levels.

“CD95” is a further example of a receptor modifying T cell functions and is also known as the Fas-receptor, which mediates apoptotic signaling by Fas-ligand expressed on the surface of other cells. CD95 has been reported to modulate TCR/CD3-driven signaling pathways in resting T lymphocytes.

A “NK Cell Specific Receptor Molecule” is, for example, CD16, a low affinity Fc receptor and NKG2D.

An example of a receptor molecule that is present on the surface of both T cells and natural killer (NK) cells is CD2 and further members of the CD2-superfamily. CD2 is able to act as a co-stimulatory molecule on T and NK cells.

A “Transmembrane Region”, in context of the present invention may be, for example, a TCR alpha or beta transmembrane domain.

A “Cytoplasmic Signalling Region” may be for example a TCR alpha or beta intracellular domain.

A “Diagnostic Agent” herein refers to a detectable molecule or substance, such as a fluorescent molecule, a radioactive molecule or any other labels known in the art that provide (either directly or indirectly) a signal.

“Fluorescent Molecules” are known in the art include fluorescein isothiocyanate (FITC), phycoerythrin (PE), fluorophores for use in the blue laser (e.g. PerCP, PE-Cy7, PE-Cy5, FL3 and APC or Cy5, FL4), fluorophores for use in the red, violet or UV laser (e.g. Pacific blue, pacific orange).

“Radioactive Molecules” include but are not limited radioactive atom for scintigraphic studies such as I¹²³, I¹²⁴, In¹¹¹, Re¹⁸⁶, Re¹⁸⁸, Tc⁹⁹. Antigen binding proteins of the invention may also comprise a spin label for nuclear magnetic resonance (NMR) imaging (also known as magnetic resonance imaging, MRI), such as iodine-123, indium-111, fluorine-19, carbon-13, nitrogen-15, oxygen-17, gadolinium, manganese or iron.

Such diagnostic agents are may be either directly coupled (i.e., physically linked) to the antigen binding protein or may be indirectly linked.

A “Therapeutic Agent” herein refers to an agent that has a therapeutic effect. In one embodiment, such a therapeutic agent may be a growth inhibitory agent, such as a cytotoxic agent or a radioactive isotope.

A “Growth Inhibitory Agent”, or “anti-proliferative agent”, which can be used indifferently, refers to a compound or composition which inhibits growth of a cell, especially a tumour cell, either in vitro or in vivo.

The term “Cytotoxic Agent” as used herein refers to a substance that inhibits or prevents the function of cells and/or causes destruction of cells. The term “Cytotoxic Agent” is intended to include chemotherapeutic agents, enzymes, antibiotics, and toxins such as small molecule toxins or enzymatically active toxins of bacterial, fungal, plant or animal origin, including fragments and/or variants thereof, and the various antitumor or anticancer agents disclosed below. In some embodiments, the cytotoxic agent is a taxoid, vincas, taxanes, a maytansinoid or maytansinoid analog such as DM1 or DM4, a small drug, a tomaymycin or pyrrolobenzodiazepine derivative, a cryptophycin derivative, a leptomycin derivative, an auristatin or dolastatin analog, a prodrug, topoisomerase II inhibitors, a DNA alkylating agent, an anti-tubulin agent, a CC-1065 or CC-1065 analog.

The term “Radioactive Isotope” is intended to include radioactive isotopes suitable for treating cancer, such as At²¹¹, Bi²¹², Er¹⁶⁹, I¹³¹, I¹²⁵, Y⁹⁰, In¹¹¹, P³², Re¹⁸⁶, Re¹⁸⁸, Sm¹⁵³, Sr⁸⁹, and radioactive isotopes of Lu. Such radioisotopes generally emit mainly beta-radiation. In an embodiment the radioactive isotope is alpha-emitter isotope, more precisely Thorium 227 which emits alpha-radiation.

In some embodiments, the antigen binding proteins of the present invention are covalently attached, directly or via a cleavable or non-cleavable linker, to the at least one growth inhibitory agent. An antigen binding protein to which such the at least one growth inhibitory agent is attached may also be referred to as a conjugate.

The preparation of such conjugates, for example immunoconjugates, is described in the application WO2004/091668 or Hudecz, F., Methods Mol. Biol. 298: 209-223 (2005) and Kirin et al., Inorg Chem. 44(15): 5405-5415 (2005), the contents of which are herein incorporated by reference in their entireties, and may by the skilled in the art be transferred to the preparation of antigen binding proteins of the present invention to which such a at least one growth inhibitory agent is attached.

“Linker” in context of the attachment of at least one growth inhibitory agent, means a chemical moiety comprising a covalent bond or a chain of atoms that covalently attaches a polypeptide to a drug moiety.

The conjugates may be prepared by in vitro methods. In order to link a drug or prodrug to the antibody, a linking group is used. Suitable linking groups are well known in the art and include disulfide groups, thioether groups, acid labile groups, photo labile groups, peptidase labile groups and esterase labile groups. Conjugation of an antigen binding protein of the invention with cytotoxic agents or growth inhibitory agents may be made using a variety of bifunctional protein coupling agents including but not limited to N-succinimidyl pyridyldithiobutyrate (SPDB), butanoic acid 4-[(5-nitro-2-pyridinyl)dithio]-2,5-dioxo-1-pyrrolidinyl ester (nitro-SPDB), 4-(Pyridin-2-yldisulfanyl)-2-sulfo-butyric acid (sulfo-SPDB), N-succinimidyl (2-pyridyldithio) propionate (SPDP), succinimidyl (N-maleimidomethyl) cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987). Carbon labeled 1-isothiocyanatobenzyl methyldiethylene triaminepentaacetic acid (MX-DTPA) is an exemplary chelating agent for conjugation of radionucleotide to the antibody (WO 94/11026).

The linker may be a “cleavable linker” facilitating release of the cytotoxic agent or growth inhibitory agent in the cell. For example, an acid-labile linker, a peptidase-sensitive linker, an esterase labile linker, a photolabile linker or a disulfide-containing linker (See e.g. U.S. Pat. No. 5,208,020) may be used. The linker may be also a “non-cleavable linker” (for example SMCC linker) that might led to better tolerance in some cases.

Alternatively, a fusion protein comprising the antibody of the invention and a cytotoxic or growth inhibitory polypeptide may be made, by recombinant techniques or peptide synthesis. The length of DNA may comprise respective regions encoding the two portions of the conjugate either adjacent one another or separated by a region encoding a linker peptide which does not destroy the desired properties of the conjugate.

The antigen binding proteins of the present invention may also be used in Dependent Enzyme Mediated Prodrug Therapy by conjugating the polypeptide to a prodrug-activating enzyme which converts a prodrug (e.g. a peptidyl chemotherapeutic agent, see WO 81/01145) to an active anti-cancer drug (See, for example, WO 88/07378 and U.S. Pat. No. 4,975,278).

A “PK Modifying Moiety” herein refers to a moiety that modifies the pharmacokinetics of the antigen binding protein of the invention. Accordingly, the moiety modifies in particular the in vivo half-life and distribution of the antigen binding protein of the invention. In a preferred embodiment, the PK modifying moiety increases the half-life of the antigen binding protein. Examples of PK modifying moieties include, but are not limited to, PEG (Dozier et al., (2015) Int J Mol Sci. October 28; 16(10):25831-64 and Jevsevar et al., (2010) Biotechnol J. January; 5(1):113-28), PASylation (Schlapschy et al., (2013) Protein Eng Des Sel. August; 26(8):489-501), albumin (Dennis et al., (2002) J Biol Chem. September 20; 277(38):35035-43), the F_{c}-part of an antibody and/or unstructured polypeptides (Schellenberger et aL, (2009) Nat Biotechnol. December; 27(12):1186-90).

In one embodiment the antigen binding protein of the invention further comprises one or more of the following: an enzyme, a cytokine (such as the human IL-2, IL-7 or IL-15), a nanocarrier, or a nucleic acid.

As mentioned herein above, in one embodiment, the antigen binding protein may be a bispecific antigen binding protein. Many different formats for antigen binding proteins that have at least two binding sites are described in the art, the antibody-based formats are, for example, diabodies, the Cross-Over-Dual-Variable-Domain (CODV) and/or in the Dual variable domain (DVD) proteins. An overview of these different bispecific antibodies and ways of making them is disclosed in, for example, Brinkmann U. and Kontermann E. E. MAbs. 2017 Feb-Mar; 9(2): 182-212. More particularly, the DVD format is, for example, disclosed in the following scientific articles (Wu C et al. Nat Biotechnol 2007; 25:1290-7; PMID:17934452; Wu C. et al. MAbs 2009; 1:339-47; Lacy S E et al. MAbs 2015; 7:605-19; PMID:25764208; Craig R B et al. PLoS One 2012; 7:e46778; PMID:23056448; Piccione E C et al. MAbs 2015). The CODV is for example disclosed in Onuoha S C et al. Arthritis Rheumatol. 2015 October; 67(10):2661-72 or for example in WO2012/135345, WO2016/116626). Bispecififc diabodies are for example described in Holliger P et al. Protein Eng 1996; 9:299-305; PMID:8736497; Atwell J L et al. Mol Immunol 1996; 33:1301-12; PMID:9171890; Kontermann R E, Nat Biotechnol 1997; 15:629-31; PMID:9219263; Kontermann R E et al. Immunotechnology 1997; 3:137-44; PMID:9237098; Cochlovius B et al. Cancer Res 2000; 60:4336-41; PMID:10969772; and DeNardo D G et al. Cancer Biother Radiopharm 2001; 16:525-35; PMID:11789029).

Techniques to produce such bispecific antibodies are also disclosed in the above cited prior art and the skilled in the art can thus easily use the CDRs or the variable domains as herein defined to generate and produce the antigen binding proteins of the invention in the herein disclosed formats.

Accordingly, the invention further refers to an antigen binding protein comprising two polypeptide chains that form two antigen binding sites (such as antigen binding site A and B), wherein a first polypeptide chain has a structure represented by the formula:

V₃-L₁-V₄-L₂-C_{L} [I]

wherein V₃ is a third variable domain; V₄ is a fourth variable domain; L₁ and L₂ are linkers; L₂ may be present or absent; C_{L} is a light chain constant domain or a portion thereof and present or absent;
and wherein a second polypeptide chain has a structure represented by the formula:

V₅-L₃-V₆-L₄-C_{H1} [II]

wherein V₅ is a fifth variable domain; V₆ is a sixth variable domain; L₃ and L₄ are linkers; L₄ may be present or absent; C_{H1} is a heavy chain constant domain 1 or a portion thereof and is present or absent; and wherein V₃ or V₄ is a first variable domain as defined herein above and V₅ or V₆ is a second variable domain as defined herein above, or
V₅ or V₆ is a first variable domain as defined in herein above and V₃ or V₄ is a second variable domain as defined herein above, and wherein
when V₃ and V₅ are variable domains as defined herein above, one of V₄ or V₆ is a light chain variable domain and the other is a heavy chain variable domain, and wherein
when V₃ and V₆ are variable domains, one of V₄ or V₅ is a light chain variable domain and the other is a heavy chain variable domain and wherein the light chain variable domain and the heavy chain variable domain form together one antigen binding site.

The linkers L₁, L₂, L₃, L₄, L₅ are defined herein above in the section ‘Definitions.’

In a related embodiment, the light chain variable domain and the heavy chain variable domain form together one antigen binding site B, and the first and second variable domain as defined herein above form one antigen binding site A.

However, in some embodiments some linker lengths might be preferable for a specific format. However, the knowledge concerning linker lengths and their amino acid sequences belongs to the general knowledge of the art, and linkers as well as linker an amino acid sequences for the different formats are part of the state of the art and are disclosed in the here above cited disclosures.

It will be understood by the skilled in the art, that the antigen binding site A, formed the first and second variable domain as defined herein above specifically binds to the PRAME peptide/MHC complex as herein described in context of the present invention.

It will be understood by the skilled in the art that, the light and heavy chain variable domains may be in a parallel orientation and the alpha and beta variable domains may be in a parallel orientation as it is the case in the DVD format, or that the light and heavy chain variable domains may be in a cross orientation and the alpha and beta variable domains may be in a cross orientation as it is the case for example in the CODV format.

Accordingly, in one embodiment, V₃ is the first variable domain and V₅ is the second variable domain as defined herein above, and V₄ is a light chain variable domain and V₆ is a heavy chain variable domain or V₄ is a heavy chain variable domain and V₆ is a light chain variable domain, or,
V₃ is the second variable domain and V₅ is the first variable domain as defined herein above, and
V₄ is a light chain variable domain and V₆ is a heavy chain variable domain or V₄ is a heavy chain variable domain and V₆ is a light chain variable domain, or,
V₃ is the first variable domain and V₆ is the second variable domain as defined herein above, and
V₄ is a light chain variable domain and V₅ is a heavy chain variable domain or V₄ is a heavy chain variable domain and V₅ is a light chain variable domain, or
V₃ is the second variable domain and V₆ is the first variable domain as defined herein above, and
V₄ is a light chain variable domain and V₅ is a heavy chain variable domain or V₄ is a heavy chain variable domain and V₅ is a light chain variable domain,
V₄ is the first variable domain and V₅ is the second variable domain as defined herein above, and
V₃ is a light chain variable domain and V₆ is a heavy chain variable domain or V₃ is a heavy chain variable domain and V₆ is a light chain variable domain, or
V₄ is the second variable domain and V₅ is the first variable domain as defined herein above, and
V₃ is a light chain variable domain and V₆ is a heavy chain variable domain or V₃ is a heavy chain variable domain and V₆ is a light chain variable domain.

In a preferred embodiment, V₃ is the first variable domain and V₆ is the second variable domain as defined in context of the present invention, and V₄ is a light chain variable domain and V₅ is a heavy chain variable domain or

V₃ is the first variable domain and V₆ is the second variable domain as defined in context of the present invention, and V₄ is a heavy chain variable domain and V₅ is a light chain variable domain.
In one embodiment, the polypeptide of formula [I] further comprises at the C-terminus the polypeptide of formula [I] a linker (L₅) and a F_{c} domain or portion thereof and/or wherein the polypeptide of formula [II] further comprises at the C-terminus of the polypeptide of formula [II] a linker (L₆) and a F_{c} domain or a portion thereof.

The F_{c}-domain is as defined herein above in the section ‘definition’.

In one embodiment the F_{c} domain comprises a N297Q mutation to remove the N-Glycosylation site within the F_{c}-part, such a mutation abrogates the F_{c}-gamma-receptor interaction. Further possible F_{c} mutations that might be employed in context of the present invention are disclosed herein below.

In one embodiment, the antigen binding protein comprises two polypeptide chains that form two antigen-binding sites (such as A and B),
wherein one polypeptide chain has a structure represented by the formula [III]:

V₃-L₁-V₄-L₂-C_{L}-L₅-F_{c1} [III]

and one polypeptide chain has a structure represented by the formula [IV]:

V₅-L₃-V₆-L₄-C_{H1}-L₆-F_{c2} [IV]

wherein V₃, L₁, V₄, L₂, C_{L}, V₅, L₃, V₆, L₄, C_{H1}, are as defined herein above, and wherein L₅ and L₆ are linkers that are present or absent and wherein F_{c1}, and F_{c2} are F_{c}-domains and wherein F_{c1} and F_{c2} are the same or different, preferably different.
The F_{c}-domain is as defined herein above in the section ‘definition’.

In one embodiment, F_{c1} comprises or consists of the amino acid sequence SEQ ID NO: 68 (hole) and F_{c2} comprises or consists of the amino acid sequence SEQ ID NO: 66 (knob), or vice versa. More preferably F_{c1} comprises or consists of the amino acid sequence SEQ ID NO: 68, when V₄ or V₃ is a heavy chain variable domain and, accordingly, F_{c2} comprises or consists of the amino acid sequence SEQ ID NO: 66, when V₅ or V₆ is a light chain variable domain, or F_{c1} comprises or consists of the amino acid sequence SEQ ID NO: 66, when V₄ or V₃ is a light chain variable domain and, accordingly, F_{c2} comprises or consists of the amino acid sequence SEQ ID NO: 68, when V₅ or V₆ is a heavy chain variable domain.

In one embodiment, the heavy chain variable domain (V_{H}) and the light chain variable domain (V_{L}) bind to an antigen selected from the group consisting of CD3 (such as the CD3γ, CD3δ, and CD3ε chains), CD4, CD7, CD8, CD10, CD11b, CD11c, CD14, CD16, CD18, CD22, CD25, CD28, CD32a, CD32b, CD33, CD41, CD41b, CD42a, CD42b, CD44, CD45RA, CD49, CD55, CD56, CD61, CD64, CD68, CD94, CD90, CD117, CD123, CD125, CD134, CD137, CD152, CD163, CD193, CD203c, CD235a, CD278, CD279, CD287, Nkp46, NKG2D, GITR, F_{c}εRI, TCRα/β and TCRγ/δ, HLA-DR and/or bind to an effector cell.

In a further embodiment, the heavy chain variable domain (V_{H}) and the light chain variable domain (V_{L}) bind to a T-cell specific receptor molecule and/or a natural killer cell (NK cell) specific receptor molecule, wherein the T-cell specific receptor molecule and the natural killer cell (NK cell) specific receptor molecule are as defined herein above.

In one embodiment, the light chain variable domain comprises or consists of the amino acid sequence SEQ ID NO: 65 and the heavy chain variable domain comprises or consists of the amino acid sequence SEQ ID NO: 171, or the light chain variable domain comprises or consists of the amino acid sequence SEQ ID NO: 65 and the heavy chain variable domain comprises or consists of the amino acid sequence SEQ ID NO: 172, or the light chain variable domain comprises or consists of the amino acid sequence SEQ ID NO: 173 and the heavy chain variable domain comprises or consists of the amino acid sequence SEQ ID NO: 174, or the light chain variable domain comprises or consists of the amino acid sequence SEQ ID NO: 175 and the heavy chain variable domain comprises or consists of the amino acid sequence SEQ ID NO: 176, or the light chain variable domain comprises or consists of the amino acid sequence SEQ ID NO: 65 and the heavy chain variable domain comprises or consists of the amino acid sequence SEQ ID NO: 177, or the light chain variable domain comprises or consists of the amino acid sequence SEQ ID NO: 65 and the heavy chain variable domain comprises or consists of the amino acid sequence SEQ ID NO: 178.
preferably, the light chain variable domain comprises or consists of the amino acid sequence SEQ ID NO: 65 and the heavy chain variable domain comprises or consists of the amino acid sequence SEQ ID NO: 171, or the light chain variable domain comprises or consists of the amino acid sequence SEQ ID NO: 65 and the heavy chain variable domain comprises or consists of the amino acid sequence SEQ ID NO: 172, or the light chain variable domain comprises or consists of the amino acid sequence SEQ ID NO: 175 and the heavy chain variable domain comprises or consists of the amino acid sequence SEQ ID NO: 176, or the light chain variable domain comprises or consists of the amino acid sequence SEQ ID NO: 65 and the heavy chain variable domain comprises or consists of the amino acid sequence SEQ ID NO: 177, or the light chain variable domain comprises or consists of the amino acid sequence SEQ ID NO: 65 and the heavy chain variable domain comprises or consists of the amino acid sequence SEQ ID NO: 178, more preferably the light chain variable domain comprises or consists of the amino acid sequence SEQ ID NO: 175 and the heavy chain variable domain comprises or consists of the amino acid sequence SEQ ID NO: 176.

As it can be seen from the examples, the inventor of the present invention demonstrated as a proof of principle the use of the TCR derived CDRs (for example the CDRs of HiAff1 and LowAff3), more particular the TCR derived variable domains, in a F_{c}-containing bispecific TCR/mAb(anti-CD3) diabody (TCER®), wherein the first polypeptide chain has a structure represented by the formula [III]:

V₃-L₁-V₄-L₂-C_{L}-L₅-F_{c1} [III]

and the second polypeptide chain has a structure represented by the formula [IV]:

V₅-L₃-V₆-L₄-C_{H1}-L₆-F_{c2} [IV]

wherein L₂, C_{L}, L₄, C_{H1}, L₅ and L₆ as defined herein above are absent and wherein V₃, L₁, V₄, F_{c1}, V₅, L₃, V₆, F_{c2} are as defined herein above.

Accordingly, in one preferred embodiment, the antigen binding protein comprises two polypeptide chains that form two antigen-binding sites, wherein one polypeptide chain has a structure represented by the formula [III]:

V₃-L₁-V₄-L₂-C_{L}-L₅-F_{c1} [III]

and one polypeptide chain has a structure represented by the formula [IV]:

V₅-L₃-V₆-L₄-C_{H1}-L₆-F_{c2} [IV]

wherein L₂, C_{L}, L₅ and L₄, C_{H1}, L₆ are absent, and
wherein V₃, L₁, V₄, V₅, L₃, V₆ are as defined herein above and wherein F_{c1}, and F_{c2} are F_{c}-domains and wherein F_{c1} and F_{c2} are different.

In the same embodiment, F_{c1} and F_{c2} preferably comprise the “knob into hole” mutation as described herein above in the section ‘definition’.

In an embodiment, preferably V₃ is the first variable domain and V₆ is the second variable domain as defined in context of the present invention, and V₄ is a light chain variable domain and V₅ is a heavy chain variable domain, and preferably, L₁ and L₃ comprise or consist of the amino acid sequence ‘GGGSGGGG’ of (SEQ ID NO: 64), and
preferably F_{c1} comprises or consists of the amino acid sequence SEQ ID NO: 66 and F_{c2} comprises or consists of the amino acid sequence SEQ ID NO: 68, or vice versa.
More preferably, F_{c1} comprises or consists of the amino acid sequence SEQ ID NO: 68, when V₄ is a heavy chain variable domain and, accordingly, F_{c2} comprises or consists of the amino acid sequence SEQ ID NO: 66, when V₅ is a light chain variable domain, or
more preferably F_{c1} comprises or consists of the amino acid sequence SEQ ID NO: 66, when V₄ is a light chain variable domain and, accordingly, F_{c2} comprises or consists of the amino acid sequence SEQ ID NO: 68, when V₅ is a heavy chain variable domain, and
the light chain variable domain and the heavy chain variable domain form together one antigen binding site preferably which binds to an antigen as defined herein above, preferably, CD3, TCRα/β or CD28, more preferably CD3 or TCRα/β,

and wherein the first and second variable domain form one antigen binding site that specifically binds to the PRAME antigenic peptide as defined in context of the present invention.

The antigen binding protein of this embodiment may be referred to as a bispecific TCR, or as a F_{c}-containing bispecific TCR/mAb diabody. The antigen binding protein of this embodiment may be also be referred to as TCER®.

“TCER@” Molecules are bispecific T-Cell Receptors (TCR) and are soluble antigen binding proteins comprising two antigen binding domains, a first antigen-binding domain formed by a first and second variable domain as defined in context of the invention and a further antigen binding domain formed by the heavy and light chain variable domain of an antibody, also referred to as recruiter, such as variable light and heavy variable domains directed against CD3 or directed against TCRα/β.

In a particular example, a newly developed so-called “F_{c}-containing HiAff1 bispecific TCR/mAb(anti-CD3) diabody” antigen binding protein comprises
one first polypeptide of formula V₃-L₁-V₄-L₂-C_{L}-L₅-F_{c1} [III] of the amino acid sequence (SEQ ID NO: 119):

QKEVEQNSGPLSVPEGAIASLNCTYSDRGSQSFFWYRQYSGKSPELIMS
IYQEGDKEDGRFTAQLNKASQYVSLLIRDSQPSDSATYLCAAVIDNDQG
GILTFGTGTRLTIIPNIQNGGGSGGGGDIQMTQSPSSLSASVGDRVTIT
CRASQDIRNYLNWYQQKPGKAPKLLIYYTSRLHSGVPSRFSGSGSGTDY
TLTISSLQPEDIATYFCQQGQTLPWTFGQGTKVEIKEPKS [symbol] DKTHTCPP
CPAP [symbol] PSVFLFPPKPKDTLMISRTPEVTCVVVDVSHEDPEVKFNWY
VDGVEVHNAKTKPREEQY [symbol] STYRVVSVLTVLHQDWLNGKEYKCKVSNKA
LPASIEKTISKAKGQPREPQVYTLPPCRDELTKNQVSLWCLVKGFYPSD
IAVEWESNGQPENNYKTTPPVLDSDGSFFLYSKLTVDKSRWQQGNVFSC
SVMHEALHNHYTQKSLSLSP

wherein L₂, C_{L}, L₅ are absent and which comprises V₃ (TCR derived alpha variable domain) of sequence SEQ ID NO: 63 (with the CDRa1, CDRa2, CDRa3 of SEQ ID NO: 11, SEQ ID NO: 17 and SEQ ID NO: 21 in bold), L₁ (underlined) of sequence SEQ ID NO: 64, V₄ (anti-CD3 VL) of sequence SEQ ID NO: 65, and F_{c1} (in italic and underlined) of sequence SEQ ID NO: 66; and
one second polypeptide of formula V₅-L₃-V₆-L₄-C_{H1}-L₆-F_{c2} [IV] of the amino acid sequence (SEQ ID NO: 120):

EVQLVQSGAEVKKPGASVKVSCKASGYSFTGYTMNWVRQAPGQGLEWMG
LINPYKGVSTYAQKFQDRVTLTVDKSTSTAYMELSSLRSEDTAVYYCAR
SGYYGDSDWYFDVWGQGTLVTVSSGGGSGGGGKAGVTQTPRYLIKTRGQ
QVTLSCSPIPGHRAVSWYQQTPGQGLQFLFEYVHGEERNKGNFPGRFSG
RQFSNSSSEMNISNLELGDSALYLCASSPWDSPNVQYFGPGTRLTVTED
LKNEPKS [symbol] DKTHTCPPCPAP [symbol] GPSVFLFPPKPKDTLMISRTPEVTCV
VVDVSHEDPEVKFNWYVDGVEVHNAKTKPREEQY [symbol] STYRVVSVLTVLHQ
DWLNGKEYKCKVSNKALPASIEKTISKAKGQPREPQVCTLPPSRDELTK
NQVSLSCAVKGFYPSDIAVEWESNGQPENNYKTTPPVLDSDGSFFLVSK
LTVDKSRWQQGNVFSCSVMHEALHNHYTQKSLSLSP

wherein L₄, C_{H1}, L₆ are absent and which comprises V₅ (anti-CD3 VH) of sequence SEQ ID NO: 67, L₃ (underlined) of sequence SEQ ID NO: 67, V₄ (TCR derived beta variable domain) of sequence SEQ ID NO: 67 (with the CDRa1, CDRa2, CDRa3 of SEQ ID NO: 45, SEQ ID NO: 46, SEQ ID NO: 48 in bold), and Fₐ (in italic and underlined) of sequence SEQ ID NO: 68, wherein the F_{c1} and F_{c2} sequence comprise a cysteine to serine mutation (S indicated in bold) since no light chain needs to be connected via said cysteine, a mutation of the IgG1 hinge region (PVA indicated in bold) to resemble the IgG2 hinge to abrogate F_{c}-gamma receptor interaction, a N297Q mutation (Q in bold) to remove the N-Glycosylation site within the F_{c}-part to abrogate F_{c}-gamma-receptor interaction and in addition the hole mutation in case of F_{c2} and the knob mutation in case of F_{c1}.
Accordingly, in one preferred embodiment, F_{c1} is of sequence SEQ ID NO: 66 and F_{c2} is of sequence SEQ ID NO: 68, or vice versa.

In one embodiment, the present invention refers to an antigen binding protein comprising a first polypeptide of formula V₃-L₁-V₄-L₂-C_{L}-L₅-F_{c1} [III] comprising or consisting of the amino acid sequence of SEQ ID NO: 119 and the second polypeptide of formula V₅-L₃-V₆-L₄-C_{H1}-L₆-F_{c2} [IV] comprising or consisting of the amino acid sequence of SEQ ID NO: 120,

It may be also desirable to modify the antigen binding protein of the present invention with respect to effector function, e.g. so as to enhance or reduce antigen-dependent cell-mediated cytotoxicity (ADCC) and/or complement dependent cytotoxicity (CDC) of the antigen binding protein. This may be achieved by introducing one or more amino acid substitutions in an F_{c} region of the antigen binding protein, herein also called F_{c}-variants in context with the antigen binding proteins of the present invention. Alternatively or additionally, cysteine residue(s) may be introduced in the F_{c} region, thereby allowing inter-chain disulfide bond formation in this region.

The homodimeric antigen binding protein thus generated may have improved or reduced internalization capability and/or increased complement-mediated cell killing and/or antibody-dependent cellular cytotoxicity (ADCC) (Caron P C. et al. 1992; and Shopes B. 1992).

Another type of amino acid modification of the antigen binding protein of the invention may be useful for altering the original glycosylation pattern of the antigen binding protein, i.e. by deleting one or more carbohydrate moieties found in the antigen binding protein, and/or adding one or more glycosylation sites that are not present in the antigen binding protein. The presence of either of the tripeptide sequences asparagine-X-serine, and asparagine-X-threonine, where X is any amino acid except proline, creates a potential glycosylation site. Addition or deletion of glycosylation sites to the antigen binding protein is conveniently accomplished by altering the amino acid sequence such that it contains one or more of the above-described tripeptide sequences (for N-linked glycosylation sites).

Another type of modification involves the removal of sequences identified, either in silico or experimentally, as potentially resulting in degradation products or heterogeneity of antigen binding protein preparations. As examples, deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in an antigen binding protein of the invention, it may therefore be desirable to remove the site, typically by conservative substitution to remove one of the implicated residues. Such substitutions in a sequence to remove one or more of the implicated residues are also intended to be encompassed by the present invention.

Another type of covalent modification involves chemically or enzymatically coupling glycosides to the antigen binding protein. These procedures are advantageous in that they do not require production of antigen binding protein in a host cell that has glycosylation capabilities for N-or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO 87/05330.

Removal of any carbohydrate moieties present on the antigen binding protein may be accomplished chemically or enzymatically. Chemical deglycosylation requires exposure of the antigen binding protein to the compound trifluoromethanesulfonic acid, or an equivalent compound. This treatment results in the cleavage of most or all sugars except the linking sugar (N-acetylglucosamine or N-acetylgalactosamine), while leaving the antigen binding protein intact. Chemical deglycosylation is described by Sojahr H. et al. (1987) and by Edge, A S. et al. (1981). Enzymatic cleavage of carbohydrate moieties on antibodies can be achieved by the use of a variety of endo-and exo-glycosidases as described by Thotakura, NR. et al. (1987).

Another type of covalent modification of the antigen binding protein comprises linking the antigen binding protein to one of a variety of non-proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

In one particular embodiment, the antigen binding protein is a TCR or a single chain TCR. In one particular embodiment, the TCR is a human TCR. Accordingly, in one embodiment, said first variable domain is comprised in a TCR α or γ chain; and/or wherein said second variable domain is comprised in a TCR β or δ chain. In one embodiment, said TCR is an alpha-beta heterodimer and comprises an alpha chain TRAC constant domain sequence and a beta chain TRBC1 or TRBC2 constant domain sequence.

The alpha chain TRAC constant domain sequence and the beta chain TRBC1 or TRBC2 constant domain are in the following, also referred to as TCR constant domain sequences. In one embodiment, the TCR constant domain sequences may be derived from any suitable species, such as any mammal, e.g., human, rat, monkey, rabbit, donkey, or mouse, preferably human. In some preferred embodiments, the TCR constant domain sequences may be slightly modified, for example, by the introduction of heterologous sequences, preferably mouse sequences, which may increase TCR expression and stability. Also, further stabilizing mutations as known from the state of the art (e.g. WO2018/104407, PCT/EP2018/069151, WO2011/044186, WO2014/018863) may be introduced, such as replacement of unfavorable amino acids in the variable regions and/or the introduction of a disulfide bridge between the TCR C domains and the removal of unpaired cysteine.

In particular, the TCR constant domain sequences may be modified by truncation or substitution to delete the native disulphide bond between Cys4 of exon 2 of TRAC and Cys2 of exon 2 of TRBC1 or TRBC2. The alpha and/or beta chain constant domain sequence(s) may also be modified by substitution of cysteine residues for Thr 48 of TRAC and Ser 57 of TRBC1 or TRBC2, the said cysteines forming a disulphide bond between the alpha and beta constant domains of the TCR. TRBC1 or TRBC2 may additionally include a cysteine to alanine mutation at position 75 of the constant domain and an asparagine to aspartic acid mutation at position 89 of the constant domain. The constant domain may additionally or alternatively contain further mutations, substitutions or deletions relative to the native TRAC and/or TRBC1/2 sequences. The term TRAC and TRBC1/2 encompasses natural polymophic variants, for example N to K at position 4 of TRAC (Bragado et al Int Immunol. 1994 February; 6(2):223-30).

In one embodiment the TCR is chimeric.

A “Chimeric TCR” herein refers to a TCR, wherein the TCR chains comprise sequences from multiple species. Preferably, a TCR in context of the invention may comprise an α chain comprising a human variable region of an α chain and, for example, a murine constant region of a murine TCR α chain.

In one embodiment the antigen binding protein of the invention is a single chain TCR (scTCR) or a single-chain bispecific antibody.

A scTCR can comprise a polypeptide of a variable region of a first TCR chain (e.g., an alpha chain) and a polypeptide of an entire (full-length) second TCR chain (e.g., a beta chain), or vice versa. Furthermore, the scTCR can optionally comprise one or more linkers which join the two or more polypeptides together. The linker can be, for instance, a peptide, which joins together two single chains, as described herein. Also provided is such a scTCR of the invention, which is fused to a human cytokine, such as IL-2, IL-7 or IL-15.

In one embodiment said single chain TCR is in one of the single chain formats selected from the group consisting of Valpha-Lₜ-Vbeta, Vbeta-Lₜ-Valpha, Valpha-Ca-Lₜ-Vbeta, Valpha-Cb-Lₜ-Vbeta, Valpha-Lₜ-Vbeta-Cb, Valpha-Lₜ-Vbeta-Ca, Valpha-Ca-Lₜ-Vbeta-Cb, Valpha-Cb-Lₜ-Vbeta-Ca, preferably Valpha-Lₜ-Vbeta, Vbeta-Lₜ-Valpha, wherein Valpha is a first variable domain as defined herein above and wherein Vbeta is a second variable domain as defined herein above, Ca and Cb are TCR alpha and beta constant regions which are present or absent respectively, and Lₜ is a linker which is present or absent and as defined herein above in the section definitions.

In one embodiment, such a single chain TCR may further comprise a further variable domain, either C-or N-terminally linked.

In one embodiment such a further variable domain may be linked via a further linker Lₖ. In one preferred embodiment, the linker Lₖ is a linker as defined herein above or a Hinge-C_{H1} sequence of the amino acid sequence SEQ ID NO: 80.

In line with the above the inventors of the present invention prepared a bispecific antigen binding protein comprising a single chain TCR, said so called “HiAFF1 bispecific Gab stabilized molecule comprising a scTCR” comprises:
one first polypeptide of the amino acid sequence (SEQ ID NO: 81)

EVQLVQSGAEVKKPGASVKVSCKASGYSFTGYTMNWVRQAPGQGLEWMGL
INPYKGVSTYAQKFQDRVTLTVDKSTSTAYMELSSLRSEDTAVYYCARSG
YYGDSDWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTSPPSPAPPVAGQKEVEQNSG
PLSVPEGAIASLNCTYSDRGSQSFFWYRQYSGKSPELIMSIYQEGDKEDG
RFTAQLNKASQYVSLLIRDSQPSDSATYLCAAVIDNDQGGILTFGTGTRL
TIIPNIQNGGGGSGGGGSGGGGSGGGGSGGGGSGSKAGVTQTPRYLIKTR
GQQVTLSCSPIPGHRAVSWYQQTPGQGLQFLFEYVHGEERNKGNFPGRFS
GRQFSNSSSEMNISNLELGDSALYLCASSPWDSPNVQYFGPGTRLTVTED
LKN

wherein said amino acid sequence comprises (in this order) an anti-CDR3 VH domain of amino acid sequence SEQ ID NO: 67, a hinge-CH, domain of amino acid sequence SEQ ID NO 80 (which is underlined), the alpha variable domain of HiAff1 of the amino acid sequence SEQ ID NO: 63, the linker L (underlined) of the amino acid sequence SEQ ID NO: 77, and a beta variable domain HiAff1 of the amino acid sequence SEQ ID NO: 83
and another polypeptide of the amino acid sequence (SEQ ID NO: 82)

DIQMTQSPSSLSASVGDRVTITCRASQDIRNYLNWYQQKPGKAPKLLIYY
TSRLHSGVPSRFSGSGSGTDYTLTISSLQPEDIATYFCQQGQTLPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

wherein said amino acid sequence comprises (in this order) an anti-CDR3 VL domain of the amino acid sequence SEQ ID NO:122 and a C_{L} domain of the amino acid sequence SEQ ID NO: 123.

Accordingly, in one particular embodiment the antigen binding protein of the invention comprises a first polypeptide of linked to a second polypeptide wherein those linked polypeptides comprise the amino acid sequence of SEQ ID NO: 81 or an amino acid sequence at least 85% identical to SEQ ID NO: 81.

The invention also includes particles displaying antigen binding protein, in particular a TCRs, and the inclusion of said particles within a library of particles. Such particles include but are not limited to phage, yeast ribosomes, or mammalian cells. Method of producing such particles and libraries are known in the art (for example see WO2004/044004; WO01/48145, Chervin et al. (2008) J. Immuno. Methods 339.2: 175-184).

### Nucleic Acids, Vectors and Recombinant Host Cells

A further object of the invention relates to an isolated nucleic acid sequence comprising or consisting of a sequence encoding an antigen binding protein as defined herein above.

Typically, said nucleic acid is a DNA or RNA molecule, which may be included in any suitable vector, such as a plasmid, cosmid, episome, artificial chromosome, phage or a viral vector.

The terms “Vector”, “Cloning Vector” and “Expression Vector” mean the vehicle by which a DNA or RNA sequence (e.g. a foreign gene) can be introduced into a host cell, so as to transform the host and promote expression (e.g. transcription and translation) of the introduced sequence.

So, a further object of the invention relates to a vector comprising a nucleic acid of the invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used, so long as a gene encoding the human antibody C region can be inserted and expressed. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, or integrative plasmids, such as for instance pUC, pcDNA, pBR, and the like.

Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a host cell which has been transfected, infected or transformed with a nucleic acid and/or a vector according to the invention.

The term “Transformation” means the introduction of a “foreign” (i.e. extrinsic) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “Transformed”.

The nucleic acids of the invention may be used to produce a recombinant antigen binding protein of the invention in a suitable expression system. The term “Expression System” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like. In some embodiments, the YB2/0 cell may be preferred, since ADCC activity of chimeric or humanized antibodies is enhanced when expressed in this cell.

According to the above, in one embodiment, the invention refers to a host cell comprising the antigen binding protein of the invention which is defined herein above, or the nucleic acid, or the vector of the invention, wherein said host cell preferably is a) a lymphocyte, such as a T lymphocyte or T lymphocyte progenitor cell, for example a CD4 or CD8 positive T cell or b) a cell for recombinant expression, such as a Chinese Hamster Ovary (CHO) cell.

In particular, for expression of some of the antigen binding proteins of the invention, in particular the antigen binding proteins comprising two polypeptides that are not linked, the expression vector may be either of a type in which a gene encoding an antibody heavy chain and a gene encoding an antibody light chain exists on separate vectors or of a type in which both genes exist on the same vector (tandem type). In respect of easiness of construction of antigen binding protein expression vector, easiness of introduction into animal cells, and balance between the expression levels of antibody H and L chains in animal cells, humanized antibody expression vector of the tandem type is preferred (Shitara K et al. J Immunol Methods. 1994 Jan. 3; 167(1-2):271-8). Examples of tandem type humanized antibody expression vector include pKANTEX93 (WO 97/10354), pEE18 and the like.

In one embodiment such recombinant host cells can be used for the production of at least one antigen binding protein of the invention

### Methods of Antigen Binding Protein of the Invention

The present invention also relates to a method of producing the antigen binding protein as defined herein above, comprising
a. providing a host cell,
b. providing a genetic construct comprising a coding sequence encoding the antigen binding protein of as defined herein above,
c. introducing said genetic construct into said suitable host cell, and
d. expressing said genetic construct by said suitable host cell, and optionally
e. selecting the cells which express and/or secrete said antibody.

In one embodiment, the method further comprises the isolation and purification of the antigen binding protein from the host cell and, optionally, reconstitution of the antigen binding protein in a T-cell.

An antigen binding protein of the invention may be produced by any technique known in the art, such as, without limitation, any chemical, biological, genetic or enzymatic technique, either alone or in combination.

In some embodiments, the antigen binding proteins of the invention have an antibody or immunoglobulin like framework.

Knowing the amino acid sequence of the desired sequence, one skilled in the art can produce antibodies or immunoglobulin chains, by standard techniques for production of polypeptides and these techniques can be transferred by the skilled in the art to the antigen binding proteins of the present invention. For instance, they can be synthesized using well-known solid phase method, in particular using a commercially available peptide synthesis apparatus (such as that made by Applied Biosystems, Foster City, Calif.) and following the manufacturer's instructions. Alternatively, antibodies, immunoglobulin chains and antigen binding proteins of the invention can be synthesized by recombinant DNA techniques as is well-known in the art. For example, fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

In one example, i.e. in case of Fe-containing bispecific TCR/mAb diabodies, DNA-sequences coding for various combinations of VH and VL and variable alpha (Valpha) and variable beta (Vbeta), as well as coding for linkers may be obtained by, for instance, gene synthesis. Resulting DNA-sequences may be cloned in frame into expression vectors coding for hinge region, CH2 and CH3 domain derived from, for example, human IgG4 [Accession #: K01316] and IgG1 [Accession #: P01857], respectively and may be further engineered. Engineering may be performed to incorporate knob-into-hole mutations into CH3-domains with and without additional interchain disulfide bond stabilization; to remove an N-glycosylation site in CH2 (e.g. N297Q mutation); to introduce F_{c}-silencing mutations or to introduce additional disulfide bond stabilization into VL and VH, respectively, according to the methods described by Reiter et al. (Stabilization of the Fv Fragments in Recombinant Immunotoxins by Disulfide Bonds Engineered into Conserved Framework Regions. Biochemistry, 1994, 33, 5451-5459).

Antigen binding proteins of the invention are suitably separated from the culture medium by immunoglobulin purification procedures such as, for example, protein A-sepharose, hydroxylapatite chromatography, gel electrophoresis, dialysis, or affinity chromatography.

In one embodiment, recovering the expressed antigen binding proteins or polypeptides herein refers to performing a protein A chromatography, a Kappa select chromatography, and/or a size exclusion chromatography, preferably a protein A chromatography and/or a size exclusion chromatography, more preferably a protein A chromatography and a size exclusion chromatography.

Methods for producing antigen binding proteins of the invention involve recombinant DNA and gene transfection techniques are well known in the art (See Morrison S L. et al. (1984) and patent documents U.S. Pat. Nos. 5,202,238; and 5,204, 244).

Methods for producing humanized antibodies based on conventional recombinant DNA and gene transfection techniques are well known in the art (See, e.g., Riechmann L. et al. 1988; Neuberger M S. et al. 1985) and can be easily applied to the production of antigen binding proteins.

In one example, vectors for the expression of the recombinant antigen binding proteins of the invention were designed as monocistronic, for instance, controlled by HCMV-derived promoter elements, pUC19-derivatives. Plasmid DNA was amplified, for example, in E.coli according to standard culture methods and subsequently purified using commercial-available kits (Macherey & Nagel). Purified plasmid DNA was used for transient transfection of, for example, CHO—S cells according to instructions of the manufacturer (ExpiCHO™ system; Thermo Fisher Scientific). Transfected CHO-cells were cultured, for instance, for 6-14 days at, for example, 32° C. to 37° C. and received one to two feeds of ExpiCHOTM Feed solution.

Conditioned cell supernatant was cleared by, for example, filtration (0.22 μm) utilizing, for instance, Sartoclear Dynamics® Lab Filter Aid (Sartorius). Bispecific antigen binding proteins were purified using, for example, an Akta Pure 25 L FPLC system (GE Lifesciences) equipped to perform affinity and size-exclusion chromatography in line. Affinity chromatography was performed on, for example, protein A or L columns (GE Lifesciences) following standard affinity chromatographic protocols. For instance, size exclusion chromatography was performed directly after elution (pH 2.8) from the affinity column to obtain highly pure monomeric protein using, for example, Superdex 200 pg 16/600 columns (GE Lifesciences) following standard protocols. Protein concentrations were determined on, for example, a NanoDrop system (Thermo Scientific) using calculated extinction coefficients according to predicted protein sequences. Concentration was adjusted, if needed, by using Vivaspin devices (Sartorius). Finally, purified molecules were stored in, for example, phosphate-buffered saline at concentrations of about 1 mg/mL at temperatures of 2-8° C.

Quality of purified bispecific antigen binding proteins was determined by, for example,

HPLC-SEC on MabPac SEC-1 columns (5 μm, 7.8×300 mm) running in, for example, 50 mM sodium-phosphate pH 6.8 containing 300 mM NaCI within a Vanquish UHPLC-System.

### Pharmaceutical Compositions

The invention further refers to a pharmaceutical composition comprising the antigen binding protein of the invention, the nucleic acids of the invention, the vector of the invention, or the host cell of the invention and a pharmaceutically acceptable carrier.

The invention also relates to an antigen binding protein according to the invention, for use as a medicament. The invention also relates to a pharmaceutical composition of the invention for use as a medicament.

The terms “Pharmaceutical Composition” or “Therapeutic Composition” as used herein refer to a compound or composition capable of inducing a desired therapeutic effect when properly administered to a subject.

In some embodiments, the subject may also be referred to as patient.

Such therapeutic or pharmaceutical compositions may comprise a therapeutically effective amount of an antigen binding protein of the invention or an antigen binding protein further comprising a therapeutic agent, in admixture with a pharmaceutically or physiologically acceptable formulation agent selected for suitability with the mode of administration.

Antigen binding protein of the present invention will usually be supplied as part of a sterile, pharmaceutical composition which will normally include a pharmaceutically acceptable carrier and/or a pharmaceutically acceptable carrier diluent.

“Pharmaceutically” or “Pharmaceutically Acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

As used herein, “Pharmaceutically-Acceptable Carriers” includes any and all solvents, dispersion media, coatings, antibacterial and antifungal agents, and the like that are physiologically compatible. Examples of suitable carriers, diluents and/or excipients include one or more of water, amino acids, saline, phosphate buffered saline, dextrose, glycerol, ethanol, and the like, as well as combination thereof. In many cases, it will be preferable to include isotonic agents, such as sugar, polyalcohol, or sodium chloride in the composition and formulation may also contain an antioxidant such as tryptamine and a stabilizing agent such as Tween 20.

A “pharmaceutically acceptable diluent,” for example, may include solvents, bulking agents, stabilizing agents, dispersion media, coatings, antibacterial and antifungal agents, isotonic and absorption delaying agents, and the like which are physiologically compatible. Examples of pharmaceutically acceptable diluents include one or more of saline, phosphate buffered saline, dextrose, glycerol, ethanol, and the like as well as combinations thereof. In many cases it will be preferable to include one or more isotonic agents, for example, sugars such as trehalose and sucrose, polyalcohol such as mannitol, sorbitol, or sodium chloride in the composition. Pharmaceutically acceptable substances such as wetting or minor amounts of auxiliary substances such as wetting or emulsifying agents, preservatives or buffers, are also within the scope of the present invention. In addition, the composition can contain excipients, such as buffers, binding agents, blasting agents, diluents, flavors, and lubricants.

The form of the pharmaceutical compositions, the route of administration, the dosage and the regimen naturally depend upon the condition to be treated, the severity of the illness, the age, weight, and gender of the patient, etc. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

Empirical considerations, such as the biological half-life, generally will contribute to the determination of the dosage. Frequency of administration may be determined and adjusted over the course of therapy and is based on reducing the number of cancer cells, maintaining the reduction of cancer cells, reducing the proliferation of cancer cells, or killing the cancer cells. Alternatively, sustained continuous release formulations of the antigen binding protein may be appropriate. Various formulations and devices for achieving sustained release are known in the art.

In one embodiment, dosages for the antigen binding proteins may be determined empirically in individuals who have been given one or more administration(s). Individuals are given incremental dosages of the antigen binding protein. To assess efficacy of the antigen binding protein, a marker of the cancer cell state can be followed. These include direct measurements of cancer cell proliferation and cell death by FACS, other imaging techniques; an improvement in health as assessed by such measurements, or an increase in quality of life as measured by accepted tests or prolongation of survival. It will be apparent to one of skill in the art that the dosage will vary depending on the individual, the stage of the disease, and the past and concurrent treatments being used.

The pharmaceutical compositions of the invention can be formulated for a topical, oral, parenteral, intranasal, intravenous, intramuscular, subcutaneous or intraocular administration and the like. Such compositions may be prepared by any method known in the art of pharmacy, for example by mixing the active ingredient with the carrier(s) or excipient(s) under sterile conditions.

In particular, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the antigen binding protein of the invention may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An antigen-binding protein of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, glycine, histidine, procaine and the like.

These salts according to the present disclosure may also include alkaline and earth alkaline salts such as salts of the Hofmeister series comprising as anions PO₄³⁻, SO₄²⁻, CH₃COO⁻, Cl⁻, Br⁻, NO₃⁻, ClO₄⁻, I⁻, SCN⁻ and as cations NH₄⁺, Rb⁺, K⁺, Na⁺, Cs⁺, Li⁺, Zn²⁺, Mg²⁺, Ca²⁺, Mn²⁺, Cu²⁺, and Ba²⁺. Particularly salts are selected from (NH₄)₃PO₄, (NH₄)₂HPO₄, (NH₄)H₂PO₄, (NH₄)₂SO₄, NH₄CH₃COO, NH₄Cl, NH₄Br, NH₄NO₃, NH₄ClO₄, NH₄I, NH₄SCN, Rb₃PO₄, Rb₂HPO₄, RbH₂PO₄, Rb₂SO₄, Rb₄CH₃COO, Rb₄Cl, Rb₄Br, Rb₄NO₃, Rb₄ClO₄, Rb₄I, Rb₄SCN, K₃PO₄, K₂HPO₄, KH₂PO₄, K₂SO₄, KCH₃COO, KCl, KBr, KNO₃, KClO₄, KI, KSCN, Na₃PO₄, Na₂HPO₄, NaH₂PO₄, Na₂SO₄, NaCH₃COO, NaCl, NaBr, NaNO₃, NaClO₄, NaI, NaSCN, ZnCl₂Cs₃PO₄, Cs₂HPO₄, CsH₂PO₄, Cs₂SO₄, CsCH₃COO, CsCl, CsBr, CsNO₃, CsClO₄, CsI, CsSCN, Li₃PO₄, Li₂HPO₄, LiH₂PO₄, Li₂SO₄, LiCH₃COO, LiCl, LiBr, LiNO₃, LiClO₄, LiI, LiSCN, Cu₂SO₄, Mg₃(PO₄)₂, Mg₂HPO₄, Mg(H₂PO₄)₂, Mg₂SO₄, Mg(CH₃COO)₂, MgCl₂, MgBr₂, Mg(NO₃)₂, Mg(ClO₄)₂, MgI₂, Mg(SCN)₂, MnCl₂, Ca₃(PO₄), Ca₂HPO₄, Ca(H₂PO₄)₂, CaSO₄, Ca(CH₃COO)₂, CaCl₂, CaBr₂, Ca(NO₃)₂, Ca(ClO₄)₂, CaI₂, Ca(SCN)₂, Ba₃(PO₄)₂, Ba₂HPO₄, Ba(H₂PO₄)₂, BaSO₄, Ba(CH₃COO)₂, BaCl₂, BaBr₂, Ba(NO₃)₂, Ba(ClO₄)₂, BaI₂, and Ba(SCN)₂. Particularly preferred are NH acetate, MgCl₂, KH₂PO₄, Na₂SO₄, KCl, NaCl, and CaCl₂, such as, for example, the chloride or acetate (trifluoroacetate) salts.

The “Carrier” can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Accordingly, in one embodiment the carrier is an aqueous carrier.

The “Aqueous Carrier” can further contain ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, salts or electrolytes, such as protamine sulfate, disodium hydrogen phosphate, dicalcium phosphate, potassium hydrogen phosphate, sodium chloride, zinc salts, colloidal silica, magnesium trisilicate, polyvinyl pyrrolidone, polyvinylpyrrolidone-vinyl acetate, cellulose-based substances (e.g., microcrystalline cellulose, hydroxypropyl methylcellulose, hydroxypropyl methylcellulose acetate succinate, hydroxypropyl methylcellulose Phthalate), starch, lactose monohydrate, mannitol, trehalose sodium lauryl sulfate, and crosscarmellose sodium, polyethylene glycol, sodium carboxymethylcellulose, polyacrylates, polymethacrylate, waxes, polyethylene-polyoxypropylene-block polymers, polyethylene glycol and wool fat.

In another aspect, the aqueous carrier is capable of imparting improved properties when combined with a peptide or other molecule described herein, for example, improved solubility, efficacy, and/or improved immunotherapy.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

The pharmaceutical formulation for systemic administration in context of the invention may be formulated for enteral, parenteral or topical administration. Indeed, all three types of formulation may be used simultaneously to achieve systemic administration of the active ingredient. Excipients as well as formulations for parenteral and non-parenteral drug delivery are set forth in Remington: The Science and Practice Of Pharmacy, 21st Edition, Lippincott Williams & Wilkins Publishing (2005), the contents which are herein incorporated by reference in their entirety.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In one embodiment, an antigen binding protein of the invention is formulated within a therapeutic mixture to comprise about 0.01 to 100 milligrams, per dose or so.

In addition antigen binding protein formulated for parenteral administration, such as intravenous or intramuscular injection, other pharmaceutically acceptable forms include, e.g. tablets or other solids for oral administration; time-release capsules; and any other form currently used.

Suitable formulations for oral administration include hard or soft gelatin capsules, pills, tablets, including coated tablets, elixirs, suspensions, syrups or inhalations and controlled release forms thereof. Generally, these agents are formulated for administration by injection (e.g., intraperitoneally, intravenously, subcutaneously, intramuscularly, etc.), although other forms of administration (e.g., oral, mucosal, etc.) can be also used. Accordingly, the antigen binding proteins are preferably combined with pharmaceutically acceptable vehicles such as saline, Ringer's solution, dextrose solution, and the like.

In certain embodiments, the use of liposomes and/or nanoparticles is contemplated for the introduction of polypeptides such as antigen binding proteins into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

### Therapeutic Methods and Uses

The inventors have shown in example 5 of the experimental section in vitro for several bi-specific compounds of the invention, such as bispecific compounds comprising the alpha and beta variable domain of HiAff1 or LoAff3 and comprising the CDR3 heavy and light chain variable domain, the cytotoxic activity of those constructs for PRAME positive cancer cell lines such as UACC257, Hs695T and U205. The inventors have furthermore demonstrated that said cytotoxic activity is highly specific and limited to PRAME-positive cells since only marginal lysis was induced by the bispecific antigen binding proteins in cell lines expressing HLA-A*02 but not presenting the peptide PRAME-004.

Furthermore, the inventors demonstrated the in vivo efficacy of the antigen binding proteins of the invention, such as a bispecific compound comprising the alpha and beta variable domain of HiAff1 and the CDR3 heavy and light chain variable domain, in pharmacodynamic studies in the hyper immune-deficient NOG mouse strain. More particularly, this mouse strain hosted the subcutaneously injected human tumor cell line HS695T and intravenously injected human peripheral blood mononuclear cell xenografts. Treatment was initiated within one hour after transplantation of human blood cells. Treatment with the bispecific compound demonstrated in vivo a strong anti-cancer activity and efficacy. The in vivo experiments are further described in the experimental section.

Accordingly, the antigen binding proteins of the present invention, in particular bispecific antigen binding proteins, such as a F_{c}-containing bispecific TCR/mAb diabodies, may be used to treat a wide variety of conditions including, for example, various forms of cancer, infections, autoimmune or inflammatory conditions, and/or fibrotic conditions. The antigen binding proteins of the present invention may be used for therapeutic purposes in humans and/or non-human mammalian animals. In one embodiment, the antigen binding proteins of the present invention can bind to tumor cells and reduce the growth of and/or kill the tumor cells presenting the peptide SLLQHLIGL (SEQ ID NO: 8)/MHC complex on their cell surface. It is understood that the antigen binding protein is administered at a concentration that promotes binding at physiological (e.g., in vivo) conditions. In another embodiment, the antigen binding proteins can be used for immunotherapy directed at tumor cells of different tissues such as colon, lung, breast, prostate, ovary, pancreas, kidney etc. In another embodiment, the antigen binding proteins alone can bind to and reduce the growth of and/or kill tumor cells.

Therefore, in one embodiment the invention provides a method of treating or preventing a proliferative disease or disorder comprising administering to a subject in need thereof a therapeutically effective amount of the antigen binding protein, the nucleic acid or vector, the host cell or the pharmaceutical composition according to the invention as defined herein above in the section “Antigen binding protein” “Nucleic acids” or “Pharmaceutical compositions”.

In one embodiment, the antigen binding protein of the invention, the nucleic acid of the invention or the vector of the invention, the host cell of the invention or the pharmaceutical composition of the invention are for use in the diagnosis, prevention, and/or treatment of a proliferative disease.

The invention further refers to the use of an antigen binding protein, the nucleic acid or vector, the host cell or the pharmaceutical composition according to the invention for the preparation of a medicament for treating or preventing a proliferative disease or disorder in a subject.

In one embodiment, the invention refers to the use of the antigen binding protein, the nucleic acid or vector, the host cell or the pharmaceutical composition according to the invention for treating or preventing a disease or disorder in a subject.

The term “Subject” or “Individual” are used interchangeably and may be, for example, a human or a non-human mammal, preferably, a human.

In the context of the invention, the term “Treating” or “Treatment”, refers to a therapeutic use (i.e. on a subject having a given disease) and means reversing, alleviating, inhibiting the progress of one or more symptoms of such disorder or condition. Therefore, treatment does not only refer to a treatment that leads to a complete cure of the disease, but also to treatments that slow down the progression of the disease and/or prolong the survival of the subject.

By “Preventing” is meant a prophylactic use (i.e. on a subject susceptible of developing a given disease).

In one embodiment, a “Disease” or “Disorder” is any condition that would benefit from treatment with the antigen binding protein of the invention. In one embodiment, this includes chronic and acute disorders or diseases including those pathological conditions which predisposes the subject to the disorder in question. The term “In Need of Treatment” refers to a subject having already the disorder as well as those in which the disorder is to be prevented.

In a particular embodiment, the antigen binding proteins of the present invention are bispecific, more particularly Fc-containing bispecific TCR/mAb diabodies as herein described.

“Proliferative Diseases”, such as cancer, involve the unregulated and/or inappropriate proliferation of cells, sometimes accompanied by destruction of adjacent tissue and growth of new blood vessels, which can allow invasion of cancer cells into new areas, i.e. metastasis. Included within conditions treatable with the antigen binding proteins are non-malignant conditions that involve inappropriate cell growth, including colorectal polyps, cerebral ischemia, gross cystic disease, polycystic kidney disease, benign prostatic hyperplasia, and endometriosis. An antigen binding protein of the invention can be used to treat a hematologic or solid tumor malignancy. More specifically, cell proliferative diseases that can be treated using the antigen binding proteins are, for example, cancers including mesotheliomas, squamous cell carcinomas, myelomas, osteosarcomas, glioblastomas, gliomas, carcinomas, adenocarcinomas, melanomas, sarcomas, acute and chronic leukemia, lymphomas, and meningioma, Hodgkin's disease, Sézary syndrome, multiple myeloma, and lung, non-small cell lung, small cell lung, laryngeal, breast, head and neck, bladder, ovarian, skin, prostate, cervical, vaginal, gastric, renal cell, kidney, pancreatic, colorectal, endometrial, esophageal, hepatobiliary, bone, skin, and hematologic cancers, as well as cancers of the nasal cavity and paranasal sinuses, the nasopharynx, the oral cavity, the oropharynx, the larynx, the hypolarynx, the salivary glands, the mediastinum, the stomach, the small intestine, the colon, the rectum and anal region, the ureter, the urethra, the penis, the testis, the vulva, the endocrine system, the central nervous system, and plasma cells.

Accordingly, in one embodiment, the proliferative disorder is cancer.

In a further embodiment, the cancer is a cancer where a PRAME-antigen is overexpressed, mutated, and/or a PRAME-derived tumor-associated antigen associated with MHC is presented.

In one preferred embodiment, said cancer is selected from the list consisting of lung cancer, such as non-small cell lung cancer, small cell lung cancer, liver cancer, head and neck cancer, skin cancer, renal cell cancer, brain cancer, gastric cancer, colorectal cancer, hepatocellular cancer, pancreatic cancer, prostate cancer, leukemia, breast cancer, Merkel cell carcinoma, melanoma, ovarian cancer, urinary bladder cancer, uterine cancer, gallbladder and bile duct cancer, and esophageal cancer, preferably ovarian carcinoma, leukemia or melanoma.

In one embodiment, whether the cancer is a cancer where a PRAME-antigen is overexpressed, mutated, and/or a PRAME-derived tumor-associated antigen associated with MHC is presented, is readily assayed for instance by using an antigen binding protein of the invention, in particular an antigen binding protein which comprises, for instance, only the first variable domain and the second variable domains as defined herein above and which is thus limited to the CDRa1, CDRa2, CDRa3, and CDRa1, CDRb2 and CDRb3 as defined herein above in context of the antigen binding proteins of the invention. Methods to identify an antigen expressing cancer using an antigen-binding protein are known to the skilled in the art.

Among the texts providing guidance for cancer therapy is Cancer, Principles and Practice of Oncology, 4th Edition, DeVita et al, Eds. J. B. Lippincott Co., Philadelphia, Pa. (1993). An appropriate therapeutic approach is chosen according to the particular type of cancer, and other factors such as the general condition of the patient, as is recognized in the pertinent field. An the antigen binding proteins of the present invention can be used by itself or can be added to a therapy regimen using other anti-neoplastic agents in treating a cancer patient.

In some embodiments, a the antigen binding protein can be administered concurrently with, before, or after a variety of drugs and treatments widely employed in cancer treatment such as, for example, chemotherapeutic agents, non-chemotherapeutic, anti-neoplastic agents, and/or radiation. For example, chemotherapy and/or radiation can occur before, during, and/or after any of the treatments described herein. Examples of chemotherapeutic agents are discussed above and include, but are not limited to, cisplatin, taxol, etoposide, mitoxantrone (Novantrone®), actinomycin D, cycloheximide, camptothecin (or water soluble derivatives thereof), methotrexate, mitomycin (e.g., mitomycin C), dacarbazine (DTIC), anti-neoplastic antibiotics such as adriamycin (doxorubicin) and daunomycin, and all the chemotherapeutic agents mentioned above.

The antigen binding proteins can also be used to treat infectious disease, for example a chronic HBV infection, an HCV infection, an HIV infection, an Epstein-Barr virus (EBV) infection, or a cytomegalovirus (CMV) infection, among many others.

The antigen binding proteins can find further use in other kinds of conditions where it is beneficial to deplete certain cell types. For example, depletion of human eosinophils in asthma, excess human B cells in systemic lupus erythematosus, excess human Th2 T cells in autoimmune conditions, or pathogen-infected cells in infectious diseases can be beneficial. In a fibrotic condition, it can be useful to deplete cells forming fibrotic tissue.

An antigen binding protein of the invention or the pharmaceutical composition thereof can be administered by itself or can be administered concurrently with, before, or after administration of other therapeutics used to treat such infectious diseases.

“Diagnosis” herein refers to a Medical diagnosis and refers to determining which disease or condition explains a person's symptoms and signs.

By a “Therapeutically Effective Amount” of the antigen binding protein or pharmaceutical composition thereof is meant a sufficient amount of the antigen binding protein to treat said proliferative disease, at a reasonable benefit/risk ratio applicable to any medical treatment. It will be understood, however, that the total daily usage of the antigen binding proteins, the nucleic acid or vector, the host cell or the pharmaceutical composition of the present invention will be decided by the attending physician within the scope of sound medical judgment. The specific therapeutically effective dose level for any particular patient will depend upon a variety of factors including the disorder being treated and the severity of the disorder; activity of the specific antigen binding protein employed; the specific composition employed, the age, body weight, general health, sex and diet of the patient; the time of administration, route of administration, and rate of excretion of the specific polypeptide employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved.

For example, the antigen binding protein of the invention, the nucleic acid of the invention or the vector of the invention, the host cell of the invention or the pharmaceutical composition of the invention can be dosed twice per week, once per week, once every two, three, four, five, six, seven, eight, nine, or ten weeks, or once every two, three, four, five, or six months. The therapeutically effective dose to be administered on each day can be from about 0.0036 mg to about 450 mg. Alternatively, the dose can calibrated according to the estimated skin surface of a patient, and each dose can be from about 0.002 mg/m² to about 250 mg/m². In another alternative, the dose can be calibrated according to a patient's weight, and each dose can be from about 0.000051 mg/kg to about 6.4 mg/kg.

In one embodiment, efficacy of the treatment with an antigen binding protein of the invention is assayed in vivo, for instance in a mouse model of cancer and by measuring, for example, changes in tumor volume between treated and control groups.

Dosages of the antigen binding protein of the present invention can vary between wide limits, depending upon the disease or disorder to be treated, the age and condition of the individual to be treated, etc.; for example, a suitable dose range for a dual specificity polypeptide molecule may be between 10 ng/kg and 100 ng/kg, preferably 25 ng/kg and 50 μg/kg. A physician will ultimately determine appropriate dosages to be used.

Pharmaceutical compositions, vectors, nucleic acids and cells of the invention may be provided in substantially pure form, for example at least 80%, at least 85%, at least 90%, at least 91%, at least 92%, at least 93%, at least 94%, at least 95%, at least 96%, at least 97%, at least 98%, at least 99% or 100% pure.

The antigen binding protein of the invention, the nucleic acid of the invention or the vector of the invention, the host cell of the invention or the pharmaceutical composition of the invention can be administered by any feasible method. Protein therapeutics will ordinarily be administered by a parenteral route, for example by injection, since oral administration, in the absence of some special formulation or circumstance, would lead to fragmentation and/or hydrolysis of the protein in the acid environment of the stomach. Subcutaneous, intramuscular, intravenous, intra-arterial, intralesional, or peritoneal bolus injection are possible routes of administration. The antigen binding protein of the invention, the host cell of the invention or the pharmaceutical composition of the invention can also be administered via infusion, for example intravenous or subcutaneous infusion. Topical administration is also possible, especially for diseases involving the skin. Alternatively, an antigen binding protein can be administered through contact with a mucus membrane, for example by intra-nasal, sublingual, vaginal, or rectal administration or administration as an inhalant. Alternatively, certain appropriate pharmaceutical compositions can be administered orally.

As herein disclosed, in some embodiments host cells as defined herein above are used in the herein described medical uses or treatment methods. In the same embodiment, the host cell is preferably a a) lymphocyte, such as a T lymphocyte or T lymphocyte progenitor cell, for example a CD4 or CD8 positive T cell, most preferably a T cell.

Accordingly, the host cell of the present invention, preferably the T cells, may be used as active ingredients of a therapeutic composition. Thus, the invention also provides a method of killing target cells in a patient whose target cells aberrantly express a polypeptide comprising the peptide SLLQHLIGL (SEQ ID NO: 8), the method comprising administering to the patient an effective number of host cells, preferably T cells. In context of this method the host cells, once administered to the subject, preferably elicit an immune response.

Accordingly, the host cell as defined herein above may be from the subject (autologous) or from another individual: preferably, said other individual is healthy.

In specific example the host cell is a T-cell. Accordingly, in context of the present invention, when a T cell as defined herein above is used as a medicament, usually, T-cells are collected from a subject by apheresis. Then the T-cells are genetically engineered to express the antigen binding protein of the present invention on their cell surface, the genetically engineered T cells are then expanded and then re-infused into the subject. In this example, the antigen binding protein is preferably a TCR.

In another approach, the host cell may be a stem cell, such as a mesenchymal stem cell and is engineered to express the antigen binding protein of the invention. In this example, the antigen binding protein is a soluble protein such as an antibody, a scTCR or a diabody as defined herein above.

Accordingly, the host cell has been transfected, infected or transformed with a nucleic acid and/or a vector according to the invention, as described herein above in the section ‘nucleic acids, vectors and recombinant host cells’.

When the host cell is transfected to express the antigen binding protein of the invention, preferably the cell comprises an expression vector capable of expressing the antigen binding protein. The host cell may then be referred to as activated host cell.

By “aberrantly expressed” the inventors also mean that the polypeptide is over-expressed compared to levels of expression in normal (healthy) tissues or that the gene is silent in the tissue from which the tumor is derived but, in the tumor, it is expressed. By “overexpressed” the inventors mean that the polypeptide is present at a level at least 1.2-fold of that present in normal tissue; preferably at least 2-fold, and more preferably at least 5-fold or 10-fold the level present in normal tissue.

Protocols for this so-called adoptive transfer of T cells are well known in the art. Reviews can be found in: Gattioni et al. and Morgan et al. (Gattinoni, L. et al., Nat. Rev. Immunol. 6 (2006): 383-393; Morgan, R. A. et al., Science 314 (2006): 126-129).

In an aspect, the TCR-elicited immune response or T-cell response may refer to the proliferation and activation of effector functions induced by a peptide, such as SLLQHLIGL (SEQ ID NO: 8), in vitro or in vivo. For MHC class I restricted cytotoxic T cells, for example, effector functions may be lysis of peptide-pulsed, peptide-precursor pulsed or naturally peptide-presenting target cells, secretion of cytokines, preferably Interferon-gamma, TNF-alpha, or IL-2 induced by peptide, secretion of effector molecules, for example, granzymes or perforins induced by peptide, or degranulation.

A number of other methods may be used for generating T cells in vitro. For example, autologous tumor-infiltrating lymphocytes can be used in the generation of CTL. Plebanski et al. (Plebanski, M. et al., Eur.J Immunol 25 (1995): 1783-1787) made use of autologous peripheral blood lymphocytes (PLBs) in the preparation of T cells. Also, B cells can be used in the production of autologous T cells.

Allogeneic cells may also be used in the preparation of T cells and a method is described in detail in U.S. Pat. No. 6,805,861, incorporated herein by reference

Host cells expressing the antigen binding protein of the invention directed against the peptides SLLQHLIGL (SEQ ID NO: 8) are useful in therapy. Thus, a further aspect of the invention provides activated host cells obtainable by the foregoing methods of the invention.

Activated host cells, which are produced by the above method, may selectively recognize a cell that aberrantly expresses a polypeptide that comprises the peptide SLLQHLIGL (SEQ ID NO: 8).

In an aspect, the host cell, in particular the T cell, recognizes the cell by interacting through its antigen binding protein, in particular its TCR, with the PRAME-004-complex (for example, binding). The host cells are useful in a method of killing target cells in a patient whose target cells aberrantly express a polypeptide comprising the peptide SLLQHLIGL (SEQ ID NO: 8) wherein the patient is administered an effective number of the activated host cells. The T cells that are administered to the patient may be derived from the patient and activated as described above (i.e. they are autologous T cells). Alternatively, the T cells are not from the patient but are from another individual. Of course, it is preferred if the individual is a healthy individual. By “healthy individual” the inventors mean that the individual is generally in good health, preferably has a competent immune system and, more preferably, is not suffering from any disease that can be readily tested for and detected.

In vivo, the target cells for the CD8-positive T cells according to the present invention can be cells of the tumor (which sometimes express MHC class II) and/or stromal cells surrounding the tumor (tumor cells) (which sometimes also express MHC class II; (Dengjel, J. et al., Clin Cancer Res 12 (2006): 4163-4170).

### Kits

Finally, the invention also provides kits comprising at least one antigen binding protein of the invention.

In one embodiment, the kit comprises
a) at least one antigen binding protein of the invention as defined herein above in the section “antigen binding proteins”,
b) optionally packaging material, and
c) optionally a label or packaging insert contained within said packaging material indicting that said antigen binding protein is effective for treating cancer or for use for the treatment of cancer.

In a related embodiment, the at least one antigen binding proteins of the invention is contained in a single and/or multi-chambered pre-filled syringes (e.g., liquid syringes and lyosyringes).

In one embodiment, the invention encompasses kits for producing a single-dose administration unit.

Accordingly, in one embodiment, the at least one antigen binding proteins of the invention as mentioned in a) of the kit of the invention is a dried antigen binding protein of the invention contained in a first container. The kit then further contains a second container having an aqueous formulation.

Accordingly, in one embodiment, the kit comprises
a) a first container comprising at least one dried antigen binding protein of the invention as defined herein above in the section “Antigen binding proteins”,
b) a second container comprising an aqueous formulation;
c) optionally packaging material, and
d) optionally a label or packaging insert contained within said packaging material indicting that said antigen binding protein is for effective for treating cancer or for use for the treatment of cancer.

The aqueous formulation is typically an aqueous solution comprising pharmaceutically-acceptable carriers as defined herein above in the section “pharmaceutical compositions”.

In a related embodiment, the “first container” and the “second” container refer to the chambers of a multi-chambered pre-filled syringes (e.g., lyosyringes).

Throughout the instant application, the term “And/Or” is a grammatical conjunction that is to be interpreted as encompassing that one or more of the cases it connects may occur. For example, the wording “such native sequence proteins can be prepared using standard recombinant and/or synthetic methods” indicates that native sequence proteins can be prepared using standard recombinant and synthetic methods or native sequence proteins can be prepared using standard recombinant methods or native sequence proteins can be prepared using synthetic methods.

Furthermore, throughout the instant application, the term “Comprising” is to be interpreted as encompassing all specifically mentioned features as well optional, additional, unspecified ones. As used herein, the use of the term “Comprising” also discloses the embodiment wherein no features other than the specifically mentioned features are present (i.e. “Consisting of”).

Furthermore the indefinite article “a” or “an” does not exclude a plurality. The mere fact that certain measures are recited in mutually different dependent claims does not indicate that a combination of these measures cannot be used to advantage.

The invention will now be described in more details with reference to the following figures and examples. All literature and patent documents cited herein are hereby incorporated by reference. While the invention has been illustrated and described in detail in the foregoing description, the examples are to be considered illustrative or exemplary and not restrictive.

### Adjuvants

Antigen binding proteins of the present invention have been shown to be capable of stimulating T cell responses (Examples 5 and 6). Thus, the antigen binding proteins of the present invention are useful for generating an immune response in a patient by which tumor cells can be destroyed. An immune response in a patient can be induced by direct administration of the described antigen binding proteins to the patient, ideally in combination with an agent enhancing the immunogenicity (i.e. an adjuvant). The immune response originating from such a therapeutic vaccination can be expected to be highly specific against tumor cells because the peptide SLLQHLIGL (SEQ ID NO: 8) is not presented or over-presented on normal tissues in comparable copy numbers, preventing the risk of undesired autoimmune reactions against normal cells in the patient.

The medicament of the invention may also include one or more adjuvants. Adjuvants are substances that non-specifically enhance or potentiate the immune response (e.g., immune responses mediated by CD8-positive T cells and helper-T (TH) cells to an antigen and would thus be considered useful in the medicament of the present invention. Suitable adjuvants include, but are not limited to, 1018 ISS, aluminum salts, AMPLIVAX®, AS15, BCG, CP-870,893, CpG7909, CyaA, dSLIM, flagellin or TLRS ligands derived from flagellin, FL T3 ligand, GM-CSF, IC30, IC31, Imiquimod (ALDARA®), resiquimod, ImuFact IMP321, Interleukins as IL-2, IL-13, IL-21, Interferon-alpha or -beta, or pegylated derivatives thereof, IS Patch, ISS, ISCOMATRIX, ISCOMs, Juvlmmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactide coglycolide) [PLG]-based and dextran microparticles, talactoferrin SRL 172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants such as Freund's or GM-CSF are preferred. Several immunological adjuvants (e.g., MF59) specific for dendritic cells and their preparation have been described previously (Allison, J. P. et al., Science 270 (1995): 932-933). Also cytokines may be used. Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12, IL-15, IL-23, IL-7, IFN-alpha. IFN-beta) (Gabrilovich, D. I. et al., Nat Med. 2 (1996): 1096-1103).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly it enhances dendritic cell maturation and differentiation, resulting in enhanced activation of TH1 cells and strong cytotoxic T-lymphocyte (CTL) generation, even in the absence of CD4 T cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG in some experiments (Krieg, A. M., Nat Rev.Drug Discov. 5 (2006): 471-484). U.S. Pat. No. 6,406,705 B1 describes the combined use of CpG oligonucleotides, non-nucleic acid adjuvants and an antigen to induce an antigen-specific immune response. A CpG TLR9 antagonist is dSLIM (double Stem Loop Immunomodulator) by Mologen (Berlin, Germany) which is a preferred component of the pharmaceutical composition of the present invention. Other TLR binding molecules such as RNA binding TLR 7, TLR 8 and/or TLR 9 may also be used.

Other examples for useful adjuvants include, but are not limited to chemically modified CpGs (e.g. CpR, Idera), dsRNA analogues such as Poly(I:C) and derivates thereof (e.g. AmpliGen®, Hiltonol®, poly-(ICLC), poly(IC-R), poly(I:C12U), non-CpG bacterial DNA or RNA as well as immunoactive small molecules and antibodies such as cyclophosphamide, sunitinib, Bevacizumab®, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g. anti-CD40, anti-TGFbeta, anti-TNFalpha receptor) and SC58175, which may act therapeutically and/or as an adjuvant. The amounts and concentrations of adjuvants and additives useful in the context of the present invention can readily be determined by the skilled artisan without undue experimentation.

Preferred adjuvants are anti-CD40, imiquimod, resiquimod, GM-CSF, cyclophosphamide, sunitinib, bevacizumab, interferon-alpha, CpG oligonucleotides and derivates, poly-(1:C) and derivates, RNA, sildenafil, and particulate formulations with PLG or virosomes.

In a preferred embodiment, the pharmaceutical composition according to the invention comprise an adjuvant wherein said adjuvant is selected from the group consisting of colony-stimulating factors, such as Granulocyte Macrophage Colony Stimulating Factor (GM-CSF, sargramostim), cyclophosphamide, imiquimod, resiquimod, and interferon-alpha.

In another preferred embodiment, the pharmaceutical composition according to the invention comprise an adjuvant wherein said adjuvant is selected from the group consisting of colony-stimulating factors, such as Granulocyte Macrophage Colony Stimulating Factor (GM-CSF, sargramostim), cyclophosphamide, imiquimod and resiquimod. In a preferred embodiment of the pharmaceutical composition according to the invention, the adjuvant is cyclophosphamide, imiquimod or resiquimod. Even more preferred adjuvants are Montanide IMS 1312, Montanide ISA 206, Montanide ISA 50V, Montanide ISA-51, polylCLC (Hiltonol®) and anti-CD40 mAB, or combinations thereof.

### Description of the Sequences

SEQ ID NO: 1 shows the amino acid sequence of the CDRa1 consensus sequence ‘DRGSQX₁’
SEQ ID NO: 2 shows the amino acid sequence of the CDRa2 consensus sequence ‘IYX₂X₃GD’
SEQ ID NO: 3 shows the amino acid sequence of the CDRa3 consensus sequence ‘CAAVIX₄NX₅X₆GGX₇LTF’
SEQ ID NO: 4 shows the amino acid sequence of the CDRb1 consensus sequence ‘X₈GHRX₉’
SEQ ID NO: 5 shows the amino acid sequence of the CDRb2 consensus sequence ‘YX₁₀X₁₁X₁₂X₁₃X₁₄’
SEQ ID NO: 6 shows the amino acid sequence of the CDRb3 consensus sequence ‘CASSPWDSPNX₁₅QYF’
SEQ ID NO: 7 shows the amino acid sequence of the full length PRAME protein, as available from the Uniprot database under accession number P78395 (as available on Jan. 11, 2019)
SEQ ID NO: 8 shows the amino acid sequence of the PRAME-004 peptide of the amino acid sequence ‘SLLQHLIGL’
SEQ ID NO: 9 shows the amino acid sequence of CDRa2 (i.e. R16P1010) of sequence ‘IYSNGD’
SEQ ID NO: 10 shows the amino acid sequence of CDRa3 (i.e. R16P1010) of sequence ‘CAAVISNFGNEKLTF’
SEQ ID NO: 11 shows the amino acid sequence of CDRa1 (i.e. HiAff1) of sequence ‘DRGSQS’
SEQ ID NO: 12 shows the amino acid sequence of CDRb1 (i.e. R16P1010) of sequence ‘SGHRS’
SEQ ID NO: 13 shows the amino acid sequence of CDRb3 (i.e. R16P1010) of sequence ‘CASSPWDSPNEQYF’
SEQ ID NO: 14 shows the amino acid sequence of CDRb2 (i.e. R16P1010) of sequence ‘YFSETQ’
SEQ ID NO: 15 shows the amino acid sequence of CDRb2 of sequence ‘YVHGX₁₆E’
SEQ ID NO: 16 shows the amino acid sequence of CDRa1 variant 1 of sequence ‘DRGSQL’
SEQ ID NO: 17 shows the amino acid sequence of CDRa2 variant 2 of sequence ‘IYQEGD’
SEQ ID NO: 18 shows the CDRa3 amino acid sequence ‘CAAVINNPSGGMLTF’
SEQ ID NO: 19 shows the CDRa3 amino acid sequence ‘CAAVIDNSNGGILTF’
SEQ ID NO: 20 shows the CDRa3 amino acid sequence ‘CAAVIDNPSGGILTF’
SEQ ID NO: 21 shows the CDRa3 amino acid sequence ‘CAAVIDNDQGGILTF’
SEQ ID NO: 22 shows the CDRa3 amino acid sequence ‘CAAVIPNPPGGKLTF’
SEQ ID NO: 23 shows the CDRa3 amino acid sequence ‘CAAVIPNPGGGALTF’
SEQ ID NO: 24 shows the CDRa3 amino acid sequence ‘CAAVIPNSAGGRLTF’
SEQ ID NO: 25 shows the CDRa3 amino acid sequence ‘CAAVIPNLEGGSLTF’
SEQ ID NO: 26 shows the CDRa3 amino acid sequence ‘CAAVIPNRLGGYLTF’
SEQ ID NO: 27 shows the CDRa3 amino acid sequence ‘CAAVIPNTDGGRLTF’
SEQ ID NO: 28 shows the CDRa3 amino acid sequence ‘CAAVIPNQRGGALTF’
SEQ ID NO: 29 shows the CDRa3 amino acid sequence ‘CAAVIPNVVGGILTF’
SEQ ID NO: 30 shows the CDRa3 amino acid sequence ‘CAAVITNIAGGSLTF’
SEQ ID NO: 31 shows the CDRa3 amino acid sequence ‘CAAVIPNNDGGYLTF’
SEQ ID NO: 32 shows the CDRa3 amino acid sequence ‘CAAVIPNGRGGLLTF’
SEQ ID NO: 33 shows the CDRa3 amino acid sequence ‘CAAVIPNTHGGPLTF’
SEQ ID NO: 34 shows the CDRa3 amino acid sequence ‘CAAVIPNDVGGSLTF’
SEQ ID NO: 35 shows the CDRa3 amino acid sequence ‘CAAVIENKPGGPLTF’
SEQ ID NO: 36 shows the CDRa3 amino acid sequence ‘CAAVIDNPVGGPLTF’
SEQ ID NO: 37 shows the CDRa3 amino acid sequence ‘CAAVIPNNNGGALTF’
SEQ ID NO: 38 shows the CDRa3 amino acid sequence ‘CAAVIPNDQGGILTF’
SEQ ID NO: 39 shows the CDRa3 amino acid sequence ‘CAAVIPNVVGGQLTF’
SEQ ID NO: 40 shows the CDRa3 amino acid sequence ‘CAAVIPNSYGGLLTF’
SEQ ID NO: 41 shows the CDRa3 amino acid sequence ‘CAAVIPNDDGGLLTF’
SEQ ID NO: 42 shows the CDRa3 amino acid sequence ‘CAAVIPNAAGGLLTF’
SEQ ID NO: 43 shows the CDRa3 amino acid sequence ‘CAAVIPNTIGGLLTF’
SEQ ID NO: 44 shows the CDRa3 amino acid sequence ‘CAAVIPNTRGGLLTF’
SEQ ID NO: 45 shows the CDRb1 (i.e. HiAff1) amino acid sequence ‘PGHRA’
SEQ ID NO: 46 shows the CDRb2 (i.e. HiAff1) amino acid sequence ‘YVHGEE’
SEQ ID NO: 47 shows the CDRb2 (i.e. Improved) amino acid sequence ‘YVHGAE’
SEQ ID NO: 48 shows the CDRb3 (i.e. HiAff1) amino acid sequence ‘CASSPWDSPNVQYF’
SEQ ID NO: 49 shows the CDRb1 (i.e. Improved) amino acid sequence ‘PGHRS’
SEQ ID NO: 50 shows the amino acid sequence of IFIT1-001 of sequence ‘VLLHHQIGL’
SEQ ID NO: 51 shows the amino acid sequence of IFT17-003 of sequence ‘FMNPHLISV’
SEQ ID NO: 52 shows the FADS2-001 of sequence ‘LLLAHIIAL’
SEQ ID NO: 53 shows the amino acid sequence of CTBP1-001 of sequence ‘ALMYHTITL’
SEQ ID NO: 54 shows the FR1-a amino acid sequence ‘QKEVEQNSGPLSVPEGAIASLNCTYS’
SEQ ID NO: 55 shows the FR2-a amino acid sequence ‘FFWYRQYSGKSPELIMS’
SEQ ID NO: 56 shows the FR3-a amino acid sequence ‘KEDGRFTAQLNKASQYVSLLIRDSQPSDSATYL’
SEQ ID NO: 57 shows the FR4-a amino acid sequence ‘GTGTRLTIIPNIQN’
SEQ ID NO: 58 shows the FR1-b amino acid sequence ‘KAGVTQTPRYLIKTRGQQVTLSCSPI’
SEQ ID NO: 59 shows the FR2-b amino acid sequence ‘VSWYQQTPGQGLQFLFE’
SEQ ID NO: 60 shows the FR3-b amino acid sequence ‘RNKGNFPGRFSGRQFSNSSSEMNISNLELGDSALYL’

SEQ ID NO: 61 shows the FR3-b amino acid sequence ‘RNKGNFPGRFSGRQFSNFSSEMNISNLELGDSALYL’
SEQ ID NO: 62 shows the FR4-b amino acid sequence ‘GPGTRLTVTEDLKN’
SEQ ID NO: 63 shows the amino acid sequence of V3 of Fc-containing HiAff1 TCR/mAb diabody of ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIDNDQGGILTFGTGTRLTIIPNIQN’
SEQ ID NO: 64 shows the amino acid sequence of Linker L₁ in the molecule Fc-containing HiAff1 TCR/mAb diabody of ‘GGGSGGGG’
SEQ ID NO: 65 shows the amino acid sequence of V_{L} of the humanized UCHT1 of sequence ‘DIQMTQSPSSLSASVGDRVTITCRASQDIRNYLNWYQQKPGKAPKLLIYYTSRLHSGVPSRFS GSGSGTDYTLTISSLQPEDIATYFCQQGQTLPWTFGQGTKVEIK’
SEQ ID NO: 66 shows the amino acid sequence of F_{c1} (comprising “Knob” mutation) of sequence
‘EPKSSDKTHTCPPCPAPPVAGPSVFLFPPKPKDTLMISRTPEVTCVVVDVSHEDPEVKFNWYV DGVEVHNAKTKPREEQYQSTYRVVSVLTVLHQDWLNGKEYKCKVSNKALPASIEKTISKAKGQ PREPQVYTLPPCRDELTKNQVSLWCLVKGFYPSDIAVEWESNGQPENNYKTTPPVLDSDGSF FLYSKLTVDKSRWQQGNVFSCSVMHEALHNHYTQKSLSLSP’
SEQ ID NO: 67 shows the amino acid sequence of the Vbeta variable domain of ‘KAGVTQTPRYLIKTRGQQVTLSCSPIPGHRAVSWYQQTPGQGLQFLFEYVHGEERNKGNFPG RFSGRQFSNSSSEMNISNLELGDSALYLCASSPWDSPNVQYFGPGTRLTVTEDLKN’
SEQ ID NO: 68 shows the amino acid sequence of F_{c2} (comprising “Hole mutation”) of sequence
‘EPKSSDKTHTCPPCPAPPVAGPSVFLFPPKPKDTLMISRTPEVTCVVVDVSHEDPEVKFNWYV DGVEVHNAKTKPREEQYQSTYRVVSVLTVLHQDWLNGKEYKCKVSNKALPASIEKTISKAKGQ PREPQVCTLPPSRDELTKNQVSLSCAVKGFYPSDIAVEWESNGQPENNYKTTPPVLDSDGSFF LVSKLTVDKSRWQQGNVFSCSVMHEALHNHYTQKSLSLSP’
SEQ ID NO: 69 shows the linker amino acid sequence ‘TVAAP’

SEQ ID NO: 70 shows the linker amino acid sequence ‘GGGS’
SEQ ID NO: 71 shows the linker amino acid sequence ‘GGGGS’
SEQ ID NO: 72 shows the linker amino acid sequence ‘TVLRT’
SEQ ID NO: 73 shows the linker amino acid sequence ‘TVSSAS’
SEQ ID NO: 74 shows the linker amino acid sequence ‘GGGGSGGGGS’
SEQ ID NO: 75 shows the linker amino acid sequence ‘GGGGSAAA’
SEQ ID NO: 76 shows the linker amino acid sequence ‘GGGGSGGGGSGGGGS’
SEQ ID NO: 77 shows the linker amino acid sequence
‘GGGGSGGGGSGGGGSGGGGSGGGGSGS’
SEQ ID NO: 78 shows the linker amino acid sequence ‘TVLSSAS’
SEQ ID NO: 79 shows the native Fc amino acid sequence (IGHG1*01)
EPKSCDKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPI EKTISKAKG QPREPQVYTLPPSRDELTKNQVSLTCLVKGFYPSDIAVEWESNGQPENNYKTTPPVLDSDGSF FLYSKLTVDKSRWQQGNVFSCSVMHEALHNHYTQKSLSLSPGK
SEQ ID NO: 80 shows the hinge-C_{H}1 amino acid sequence
ASTKG PSVFPLAPSSKSTSGGTAALGCLVKDYFPEPVTVSWNSGALTSGVHTFPAVLQSSGLY SLSSVVTVPSSSLGTQTYICNVNHKPSNTKVDKKVEPKSCDKTHTSPPSPAPPVAG
SEQ ID NO: 81 shows the amino acid sequence of the first polypeptide of the scTCR-Fab construct of the sequence
‘EVQLVQSGAEVKKPGASVKVSCKASGYSFTGYTMNWVRQAPGQGLEWMGLINPYKGVSTYA QKFQDRVTLTVDKSTSTAYMELSSLRSEDTAVYYCARSGYYGDSDWYFDVWGQGTLVTVSSA STKGPSVFPLAPSSKSTSGGTAALGCLVKDYFPEPVTVSWNSGALTSGVHTFPAVLQSSGLYS LSSVVTVPSSSLGTQTYICNVNHKPSNTKVDKKVEPKSCDKTHTSPPSPAPPVAGQKEVEQNS GPLSVPEGAIASLNCTYSDRGSQSFFWYRQYSGKSPELIMSIYQEGDKEDGRFTAQLNKASQY VSLLIRDSQPSDSATYLCAAVIDNDQGGILTFGTGTRLTIIPNIQNGGGGSGGGGSGGGGSGGG GSGGGGSGSKAGVTQTPRYLIKTRGQQVTLSCSPIPGHRAVSWYQQTPGQGLQFLFEYVHGE ERNKGNFPGRFSGRQFSNSSSEMNISNLELGDSALYLCASSPWDSPNVQYFGPGTRLTVTED LKN’
SEQ ID NO: 82 shows the amino acid sequence of the second polypeptide of the scTCR-Fab construct of sequence
‘DIQMTQSPSSLSASVGDRVTITCRASQDIRNYLNWYQQKPGKAPKLLIYYTSRLHSGVPSRFS GSGSGTDYTLTISSLQPEDIATYFCQQGQTLPWTFGQGTKVEIKRTVAAPSVFIFPPSDEQLKS GTASVVCLLNNFYPREAKVQWKVDNALQSGNSQESVTEQDSKDSTYSLSSTLTLSKADYEKH KVYACEVTHQGLSSPVTKSFNRGEC’
SEQ ID NO: 83 shows the amino acid sequence of Vbeta-domain (designated B2) of sequence ‘KAGVTQTPRYLIKTRGQQVTLSCSPIPGHRAVSWYQQTPGQGLQFLFEYVHGEERNKGNFPG RFSGRQFSNSSSEMNISNLELGDSALYLCASSPWDSPNVQYFGPGTRLTVTEDLKN’
SEQ ID NO: 84 shows the amino acid sequence of Linker and Myc tag of sequence ‘AAAGGSGGEQKLISEEDL’
SEQ ID NO: 85 shows the amino acid sequence of Valpha A_HiAff1#1 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNPGGGALTFGTGTRLTIIPNIQN’
SEQ ID NO: 86 shows the amino acid sequence of Valpha A_HiAff1#2 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNSAGGRLTFGTGTRLTIIPNIQN’
SEQ ID NO: 87 shows the amino acid sequence of Valpha A_HiAff1#3 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNLEGGSLTFGTGTRLTIIPNIQN’
SEQ ID NO: 88 shows the amino acid sequence of Valpha A_HiAff1#4 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVI PNRLGGYLTFGTGTRLTIIPNIQN’
SEQ ID NO: 89 shows the amino acid sequence of Valpha A_HiAff1#5 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNTDGGRLTFGTGTRLTIIPNIQN’
SEQ ID NO: 90 shows the amino acid sequence of Valpha A_HiAff1#6 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNQRGGALTFGTGTRLTIIPNIQN’
SEQ ID NO: 91 shows the amino acid sequence of Valpha A_HiAff1#7 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNVVGGILTFGTGTRLTIIPNIQN’
SEQ ID NO: 92 shows the amino acid sequence of Valpha A_HiAff1#9 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVITNIAGGSLTFGTGTRLTIIPNIQN’
SEQ ID NO: 93 shows the amino acid sequence of Valpha A_HiAff1#10 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNDQGGYLTFGTGTRLTIIPNIQN’
SEQ ID NO: 94 shows the amino acid sequence of Valpha A_HiAff1#11 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNGRGGLLTFGTGTRLTIIPNIQN’
SEQ ID NO: 95 shows the amino acid sequence of Valpha A_HiAff1#12 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNTHGGPLTFGTGTRLTIIPNIQN’
SEQ ID NO: 96 shows the amino acid sequence of Valpha A_HiAff1#13 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNDVGGSLTFGTGTRLTIIPNIQN’
SEQ ID NO: 97 shows the amino acid sequence of Valpha A_HiAff1#14 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIENKPGGPLTFGTGTRLTIIPNIQN’
SEQ ID NO: 98 shows the amino acid sequence of Valpha A_HiAff1#15 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIDNPVGGPLTFGTGTRLTIIPNIQN’
SEQ ID NO: 99 shows the amino acid sequence of Valpha A_HiAff1#16 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNNIGGALTFGTGTRLTIIPNIQN’
SEQ ID NO: 100 shows the amino acid sequence of Valpha A_HiAff1#17 of sequence ‘QKEVEQNSGPLSVPEGAIASLNCTYSDRGSQSFFWYRQYSGKSPELIMSIYQEGDKEDGRFT AQLNKASQYVSLLIRDSQPSDSATYLCAAVIPNDQGGILTFGTGTRLTIIPNIQN’
SEQ ID NO: 101 shows the amino acid sequence of Valpha A_HiAff1#18 of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNDQGGILTFGTGTRLTIIPNIQN’
SEQ ID NO: 102 shows the amino acid sequence of Valpha A_HiAff1#19 of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNSAGGRLTFGTGTRLTIIPNIQN’
SEQ ID NO: 103 shows the amino acid sequence of Valpha A_HiAff1#20 of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNRLGGYLTFGTGTRLTIIPNIQN’
SEQ ID NO: 104 shows the amino acid sequence of Valpha A_HiAff1#22 of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNTHGGPLTFGTGTRLTIIPNIQN’
SEQ ID NO: 105 shows the amino acid sequence of Valpha A_HiAff1#23 of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNVVGGQLTFGTGTRLTIIPNIQN’
SEQ ID NO: 106 shows the amino acid sequence of Valpha A_HiAff1#24 of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNSYGGLLTFGTGTRLTIIPNIQN’
SEQ ID NO: 107 shows the amino acid sequence of Valpha A_HiAff1#26 of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNAAGGLLTFGTGTRLTIIPNIQN’
SEQ ID NO: 108 shows the amino acid sequence of Valpha A_HiAff1#27 of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNTIGGLLTFGTGTRLTIIPNIQN’
SEQ ID NO: 109 shows the amino acid sequence of Valpha A_HiAff1#29 of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNDDGGRLTFGTGTRLTIIPNIQN’
SEQ ID NO: 110 shows the amino acid sequence of Valpha A_HiAff1#30 of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNTRGGLLTFGTGTRLTIIPNIQN’
SEQ ID NO: 111 shows the amino acid sequence of Valpha A_HiAff1_38 of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIDNDQGGILTFGTGTRLTIIPNIQN’
SEQ ID NO: 112 shows the amino acid sequence of Vbeta B2_S85F of sequence KAGVTQTPRYLIKTRGQQVTLSCSPIPGHRAVSWYQQTPGQGLQFLFEYVHGEERNKGNFPG RFSGRQFSNFSSEMNISNLELGDSALYLCASSPWDSPNVQYFGPGTRLTVTEDLKN
SEQ ID NO: 113 shows the amino acid sequence of Valpha A_HiAff1#24 of sequence QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNSYGGLLTFGTGTRLTIIPNIQN
SEQ ID NO: 114 shows the amino acid sequence of Valpha A_HiAff1#18 of sequence QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNDQGGILTFGTGTRLTIIPNIQN
SEQ ID NO: 115 shows the amino acid sequence of Valpha A_HiAff1#23 of sequence QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNVVGGQLTFGTGTRLTIIPNIQN
SEQ ID NO: 116 shows the amino acid sequence of Valpha A_HiAff1#20 of sequence QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNRLGGYLTFGTGTRLTIIPNIQN
SEQ ID NO: 117 shows a linker amino acid sequence GGGGSGGGGSGGGGSGGGGS
SEQ ID NO: 118 shows the amino acid sequence of Valpha A_HiAff1-38 of sequence QKEVEQNSGPLSVPEGAIASLNCTYSDRGSQLFFWYRQYSGKSPELIMSIYQEGDKEDGRFTA QLNKASQYVSLLIRDSQPSDSATYLCAAVIDNDQGGILTFGTGTRLTIIPNIQN
SEQ ID NO: 119 shows the amino acid sequence of the first polypeptide of Fc-containing HiAff1 bispecific TCR/mAb(anti-CD3) diabody of sequence QKEVEQNSGPLSVPEGAIASLNCTYSDRGSQSFFWYRQYSGKSPELIMSIYQEGDKEDGRFTA QLNKASQYVSLLIRDSQPSDSATYLCAAVIDNDQGGILTFGTGTRLTIIPNIQNGGGSGGGGDIQ MTQSPSSLSASVGDRVTITCRASQDIRNYLNWYQQKPGKAPKLLIYYTSRLHSGVPSRFSGSG SGTDYTLTISSLQPEDIATYFCQQGQTLPWTFGQGTKVEIKEPKSSDKTHTCPPCPAPPVAGPS VFLFPPKPKDTLMISRTPEVTCVVVDVSHEDPEVKFNWYVDGVEVHNAKTKPREEQYQSTYRV VSVLTVLHQDWLNGKEYKCKVSNKALPASIEKTISKAKGQPREPQVYTLPPCRDELTKNQVSL WCLVKGFYPSDIAVEWESNGQPENNYKTTPPVLDSDGSFFLYSKLTVDKSRWQQGNVFSCSV MHEALHNHYTQKSLSLSP
SEQ ID NO: 120 shows the amino acid sequence of the second polypeptide of Fc-containing HiAff1 bispecific TCR/mAb(anti-CD3) diabody of sequence EVQLVQSGAEVKKPGASVKVSCKASGYSFTGYTMNWVRQAPGQGLEWMGLINPYKGVSTYA QKFQDRVTLTVDKSTSTAYMELSSLRSEDTAVYYCARSGYYGDSDWYFDVWGQGTLVTVSS GGGSGGGGKAGVTQTPRYLIKTRGQQVTLSCSPIPGHRAVSWYQQTPGQGLQFLFEYVHGE ERNKGNFPGRFSGRQFSNSSSEMNISNLELGDSALYLCASSPWDSPNVQYFGPGTRLTVTED LKNEPKSSDKTHTCPPCPAPPVAGPSVFLFPPKPKDTLMISRTPEVTCVVVDVSHEDPEVKFN WYVDGVEVHNAKTKPREEQYQSTYRVVSVLTVLHQDWLNGKEYKCKVSNKALPASIEKTISKA KGQPREPQVCTLPPSRDELTKNQVSLSCAVKGFYPSDIAVEWESNGQPENNYKTTPPVLDSD GSFFLVSKLTVDKSRWQQGNVFSCSVMHEALHNHYTQKSLSLSP
SEQ ID NO: 121 shows the amino acid sequence of VH of anti-CD3 of sequence DIQMTQSPSSLSASVGDRVTITCRASQDIRNYLNWYQQKPGKAPKLLIYYTSRLHSGVPSRFSG SGSGTDYTLTISSLQPEDIATYFCQQGQTLPWTFGQGTKVEIKR
SEQ ID NO: 122 shows the amino acid sequence of VL of anti-CD3 of sequence DIQMTQSPSSLSASVGDRVTITCRASQDIRNYLNWYQQKPGKAPKLLIYYTSRLHSGVPSRFSG SGSGTDYTLTISSLQPEDIATYFCQQGQTLPWTFGQGTKVEIKR
SEQ ID NO: 123 shows the amino acid sequence of C_{L} of sequence TVAAPSVFIFPPSDEQLKSGTASVVCLLNNFYPREAKVQWKVDNALQSGNSQESVTEQDSKDS TYSLSSTLTLSKADYEKHKVYACEVTHQGLSSPVTKSFNRGEC
SEQ ID NO: 124 shows the amino acid sequence of alpha beta fusion of sequence QKEVEQNSGPLSVPEGAIASLNCTYSDRGSQSFFWYRQYSGKSPELIMSIYQEGDKEDGRFTA QLNKASQYVSLLIRDSQPSDSATYLCAAVIDNDQGGILTFGTGTRLTIIPNIQNGGGGSGGGGS GGGGSGGGGSGGGGSGSKAGVTQTPRYLIKTRGQQVTLSCSPIPGHRAVSWYQQTPGQGL QFLFEYVHGEERNKGNFPGRFSGRQFSNSSSEMNISNLELGDSALYLCASSPWDSPNVQYFG PGTRLTVTEDLKN
SEQ ID NO: 125 shows the amino acid sequence of Peptide ATP1A1-001 of sequence FLPIHLLGL
SEQ ID NO: 126 shows the amino acid sequence of Peptide NADK-002 of sequence YLDGHLITT
SEQ ID NO: 127 shows the amino acid sequence of Peptide ITSN1-001 of sequence ILAMHLIDV
SEQ ID NO: 128 shows the amino acid sequence of Peptide SMA-003 of sequence IVINHVISV
SEQ ID NO: 129 shows the amino acid sequence of Peptide VPS13A-001 of sequence ILQPHVIAL
SEQ ID NO: 130 shows the amino acid sequence of Peptide SF3B3-005 of sequence TLVYHVVGV
SEQ ID NO: 131 shows the amino acid sequence of Peptide NCAM2-001 of sequence FVQPHIIQL
SEQ ID NO: 132 shows the amino acid sequence of Peptide KNT-001 of sequence YMLEHVITL
SEQ ID NO: 133 shows the amino acid sequence of Peptide EHD4-001 of sequence ALAKHLIKI
SEQ ID NO: 134 shows the amino acid sequence of Peptide EHD-001 of sequence ALANHLIKV
SEQ ID NO: 135 shows the amino acid sequence of Peptide MCMB-002 of sequence YLILHLIST
SEQ ID NO: 136 shows the amino acid sequence of Peptide HSPAS-001 of sequence RVMEHFIKL
SEQ ID NO: 137 shows the amino acid sequence of Peptide CEBPZ-002 of sequence KLYQHEINL
SEQ ID NO: 138 shows the amino acid sequence of Peptide DCAF12-001 of sequence SFYEHIITV
SEQ ID NO: 139 shows the amino acid sequence of Peptide EHD2-002 of sequence ALASHLIEA
SEQ ID NO: 140 shows the amino acid sequence of Peptide KIAA1324-001 of sequence SLADRLIGV
SEQ ID NO: 141 shows the amino acid sequence of Peptide MYO1A-001 of sequence LVSEHVIEL
SEQ ID NO: 142 shows the amino acid sequence of Peptide SERPINA6-001 of sequence SLYKHLVAL
SEQ ID NO: 143 shows the amino acid sequence of Peptide SMARCD1-001 of sequence IIINHVISV
SEQ ID NO: 144 shows the amino acid sequence of Peptide TSC1-001 of sequence SLLGHVIRL
SEQ ID NO: 145 shows the amino acid sequence of Peptide UGT-005 of sequence SLIEHMIML
SEQ ID NO: 146 shows the amino acid sequence of Peptide VAV1-001 of sequence NLLPHAINL
SEQ ID NO: 147 shows the amino acid sequence of Peptide WDFY3-004 of sequence SLFEHFIEL
SEQ ID NO: 148 shows the amino acid sequence of R16P1C10 alpha variable domain of sequence MKSLRVLLVILWLQLSWVWSQQKEVEQNSGPLSVPEGAIASLNCTYSDRGSQSFFWYRQYSG KSPELIMFIYSNGDKEDGRFTAQLNKASQYVSLLIRDSQPSDSATYLCAAVISNFGNEKLTFGTG TRLTIIP
SEQ ID NO: 149 shows the amino acid sequence of R16P1010 beta variable domain of sequence MGSRLLCWVLLCLLGAGPVKAGVTQTPRYLIKTRGQQVTLSCSPISGHRSVSWYQQTPGQGL QFLFEYFSETQRNKGNFPGRFSGRQFSNSRSEMNVSTLELGDSALYLCASSPWDSPNEQYFG PGTRLTVT
SEQ ID NO: 150 shows the amino acid sequence of scTCR CDR6 of sequence QKEVEQNSGPLSVPEGAIASLNCTYSDRGSQSFFWYRQYSGKSPELIMSIYSNGDKEDGRFTA QLNKASQYVSLLIRDSQPSDSATYLCAAVIDNDQGGILTFGTGTRLTIIPNIQNGGGGSGGGGS GGGGSGGGGSGGGGSGVTQTPRYLIKTRGQQVTLSCSPIPGHRAVSWYQQTPGQGLQFLFE YVHGEERNKGNFPGRFSGRQFSNSSSEMNISNLELGDSALYLCASSPWDSPNVQYFGPGTRL TVTEDLKN
SEQ ID NO: 151 shows the amino acid sequence of a linker of sequence GGGGSGGGGSGGGGSGGGGSGGGGS
SEQ ID NO: 152 shows the amino acid sequence of the Aga2p yeast mating protein together with its leader sequence of sequence MQLLRCFSIFSVIASVLAQELTTICEQIPSPTLESTPYSLSTTTILANGKAMQGVFEYYKSVTFVS NCGSHPSTTSKGSPINTQYVF
SEQ ID NO: 153 shows the amino acid sequence of the fusion protein Leader peptide-Aga2p-FLAG-L-scTCR CDR6-L-Myc of sequence MQLLRCFSIFSVIASVLAQELTTICEQIPSPTLESTPYSLSTTTILANGKAMQGVFEYYKSVTFVS NCGSHPSTTSKGSPINTQYVFGGGGSDYKDDDDKGGGASQKEVEQNSGPLSVPEGAIASLNC TYSDRGSQSFFWYRQYSGKSPELIMSIYSNGDKEDGRFTAQLNKASQYVSLLIRDSQPSDSAT YLCAAVIDNDQGGILTFGTGTRLTIIPNIQNGGGGSGGGGSGGGGSGGGGSGGGGSGVTQTP RYLIKTRGQQVTLSCSPIPGHRAVSWYQQTPGQGLQFLFEYVHGEERNKGNFPGRFSGRQFS NSSSEMNISNLELGDSALYLCASSPWDSPNVQYFGPGTRLTVTEDLKNAAAGGSGGEQKLISE EDL
SEQ ID NO: 154 shows the amino acid sequence of the FLAG tag plus linkers of sequence GGGGSDYKDDDDKGGGASSEQ ID NO: 155 shows the hinge region IgG1 sequence E₂₁₆PKSCDKTHTCPPCPAPELLG
SEQ ID NO: 156 shows the hinge region IgG2 sequence E₂₁₆RKCCVECPPCPAPPVAGP
SEQ ID NO: 157 shows the hinge region IgG3 sequence ELKTPLGDTTHTCPRCPEPKSCDTPPPCPRCPE₂₁₆PKSCDTPPPCPRCPAPELLG
SEQ ID NO: 158 shows the hinge region IgG4 sequence E₂₁₆SKYGPPCPSCPAPEFLG
SEQ ID NO: 159 shows the linker sequence GGGGSGT
SEQ ID NO: 160 shows the linker sequence GGSGGGGSGG
SEQ ID NO: 161 shows the linker sequence GGSGGGGSGGGGSGG
SEQ ID NO: 162 shows the linker sequence GGGGSGGGGSGT
SEQ ID NO: 163 shows the linker sequence GGGGSGGGGSGGGGSGT
SEQ ID NO: 164 shows the linker sequence GGGGSGGGGSGGGGSGGGGSGT
SEQ ID NO: 165 shows the linker sequence GGSGGGGSGGGGSGGGGSGG
SEQ ID NO: 166 shows the linker sequence GGGGSGGGGSGGGGSGGGGSGGGGSGT
SEQ ID NO: 167 shows the linker sequence GGSGGGGSGGGGSGGGGSGGGGSGG
SEQ ID NO: 168 shows the linker sequence GSADDAKKDAAKKDGKS
SEQ ID NO: 169 shows the linker sequence GGQGSGGTGSGGQGSGGTGSGGQGS
SEQ ID NO: 170 shows the linker sequence GGGGSGGGGSGGGGSGGGGS
SEQ ID NO: 171 shows the UCHT1(17) VH sequence EVQLVQSGAEVKKPGASVKVSCKASGYSFTGYTMNWVRQAPGQGLEWMGLINPYKGVSTYA QKFQDRVTLTVDKSTSTAYMELSSLRSEDTAVYYCARSGYYGDSDWYFDVWGQGTLVTVSS
SEQ ID NO: 172 shows the UCHT1(23) VH sequence EVQLVQSGAEVKKPGASVKVSCKASGYSFTEYTMNWVRQAPGQGLEWMGLINPQKGVSTYA QKFQDRVTLTVDKSTSTAYMELSSLRSEDTAVYYCARSGYYGDSDWYFDVWGQGTLVTVSS
SEQ ID NO: 173 shows the BMA (10) VL sequence QIQMTQSPSSLSASVGDRVTITCSATSSVSYMHWYQQKPGKAPKRWIYDTSKLASGVPSRFSG SGSGTDYTLTISSLQPEDAATYYCQQWSSNPLTFGGGTKVEIK
SEQ ID NO: 174 shows the BMA (10) VH sequence EVQLVQSGAEVKKPGASVKVSCKASGYKFTSYVMHWVRQAPGQGLEWMGYINPYNDVTKYA EKFGGRVTLTSDTSTSTAYMELSSLRSEDTAVHYCARGSYYDYDGFVYWGQGTLVTVSS
SEQ ID NO: 175 shows the BMA(36) VL sequence QIQMTQSPSSLSASVGDRVTITCSATSSVSYMHWYQQKPGKAPKRWIYDTSKLASGVPSRFSG SGSGTDYTLTISSLQPEDAATYYCQQWSSNPLTFGGGTKVEIK
SEQ ID NO: 176 shows the BMA(36) VH sequence EVQLVQSGAEVKKPGASVKVSCKASGYKFTSYVMHWVRQAPGQGLEWMGYINPYNDVTKYA EKFQGRVTLTSDTSTSTAYMELSSLRSEDTAVHYCARGSYYDYEGFVYWGQGTLVTVSS
SEQ ID NO: 177 shows the UCHT1 (17opt) VH sequence EVQLVQSGAEVKKPGASVKVSCKASGYSFTGYTMNWVRQAPGQGLEWMGLINPYKGVSTYA QKFQDRVTLTVDKSTSTAYMELSSLRSEDTAVYYCARSGYYGESDWYFDVWGQGTLVTVSS
SEQ ID NO: 178 shows the UCHT1 (21) VH sequence EVQLVQSGAEVKKPGASVKVSCKASGYSFTGYTMNWVRQAPGQGLEWMGLINPQKGVSTYA QKFQDRVTLTVDKSTSTAYMELSSLRSEDTAVYYCARSGYYGDSDWYFDVWGQGTLVTVSS
SEQ ID NO: 179 shows the CDRa3 amino acid sequence ‘CAAVIPNDDGGRLTF’ of SEQ ID NO: 109.
SEQ ID NO: 180 shows the amino acid sequence of Valpha A_HiAff1#29B of sequence ‘QKEVEQNSGPLSVPEGAIASLNCTYSDRGSQLFFWYRQYSGKSPELIMSIYQEGDKEDGRFTA QLNKASQYVSLLIRDSQPSDSATYLCAAVIPNDDGGLLTFGTGTRLTIIPNIQN
SEQ ID NO: 181 shows the CDRa3 amino acid sequence ‘CAAVIPNDQGGYLTF’ of SEQ ID NO: 93;
SEQ ID NO: 182 shows the CDRa3 amino acid sequence ‘CAAVIPNNIGGALTF’ of SEQ ID NO: 99.

### EXAMPLES

Native T cell receptors (TCRs) against cancer antigens are often of lower affinity when compared to TCRs targeting viral antigens, and this may be one possible explanation for tumor immune escape (Aleksic et al. 2012). Therefore, it is desirable to have higher affinity TCR variants designed for the use as antigen recognizing constructs as recognition module of a soluble approach, i.e. using bispecific molecules or in an adoptive cell therapy (Hickman et al. 2016). This invention thus relates to the modification and optimization of the naturally occurring T cell receptor R16P1010 targeting the tumor associated peptide PRAME-004 (SEQ ID NO: 8), wherein said T cell receptor R16P1C10 comprises an alpha variable domain comprising the amino acid sequence of SEQ ID NO: 148 and a beta variable domain comprising the amino acid sequence of SEQ ID NO: 149 and which is disclosed in WO2018/172533.

### Example 1

### Design of Soluble Bispecific TCR Molecules

TCRs consisting of Valpha and Vbeta domains were designed, produced and tested either in a single-chain (scTCR) format coupled to a Fab-fragment or in an F_{c}-containing bispecific TCR/mAb diabody format. In case of F_{c}-containing bispecific TCR/mAb diabodies DNA-sequences coding for various combinations of VH and VL, derived from hUCHT1(Var17), a newly humanized version of the antiCD3-antibody UCHT1, and Valpha and Vbeta as well as sequences coding for linkers were obtained by gene synthesis. Resulting DNA-sequences were cloned in frame into expression vectors coding for hinge region, C_{H2} and C_{H3} domain derived from human IgG1 [Accession #: P01857] and were further engineered. Engineering was performed to incorporate knob-into-hole mutations into CH3-domains with additional interchain disulfide bond stabilization; to remove an N-glycosylation site in CH2 (e.g. N297Q mutation); to introduce Fe-silencing mutations.

### Example 2

### Production and Purification of Soluble Bispecific Molecules

Vectors for the expression of recombinant proteins were designed as monocistronic, controlled by HCMV-derived promoter elements, pUC19-derivatives. Plasmid DNA was amplified in E.coli according to standard culture methods and subsequently purified using commercial-available kits (Macherey & Nagel). Purified plasmid DNA was used for transient transfection of CHO—S cells according to instructions of the manufacturer (ExpiCHO™ system; Thermo Fisher Scientific). Transfected CHO-cells were cultured for 6-14 days at 32° C. to 37° C. and received one to two feeds of ExpiCHO™ Feed solution. Conditioned cell supernatant was cleared by filtration (0.22 μm) utilizing Sartoclear Dynamics® Lab Filter Aid (Sartorius). Bispecific molecules were purified using an Akta Pure 25 L FPLC system (GE Lifesciences) equipped to perform affinity and size-exclusion chromatography in line. Affinity chromatography was performed on protein A or L columns (GE

Lifesciences) following standard affinity chromatographic protocols. Size exclusion chromatography was performed directly after elution (pH 2.8) from the affinity column to obtain highly pure monomeric protein using Superdex 200 pg 16/600 columns (GE Lifesciences) following standard protocols. Protein concentrations were determined on a NanoDrop system (Thermo Scientific) using calculated extinction coefficients according to predicted protein sequences. Concentration was adjusted, if needed, by using Vivaspin devices (Sartorius). Finally, purified molecules were stored in phosphate-buffered saline at concentrations of about 1 mg/mL at temperatures of 2-8° C. Quality of purified bispecific molecules was determined by HPLC-SEC on MabPac SEC-1 columns (5 μm, 7.8×300 mm) running in 50 mM sodium-phosphate pH 6.8 containing 300 mM NaCl within a Vanquish UHPLC-System. Non-reducing and reducing SDS-PAGE confirmed the purity and expected size of the different dual specificity TCR/mAb molecules.

### Example 3

### Stress Stability Testing

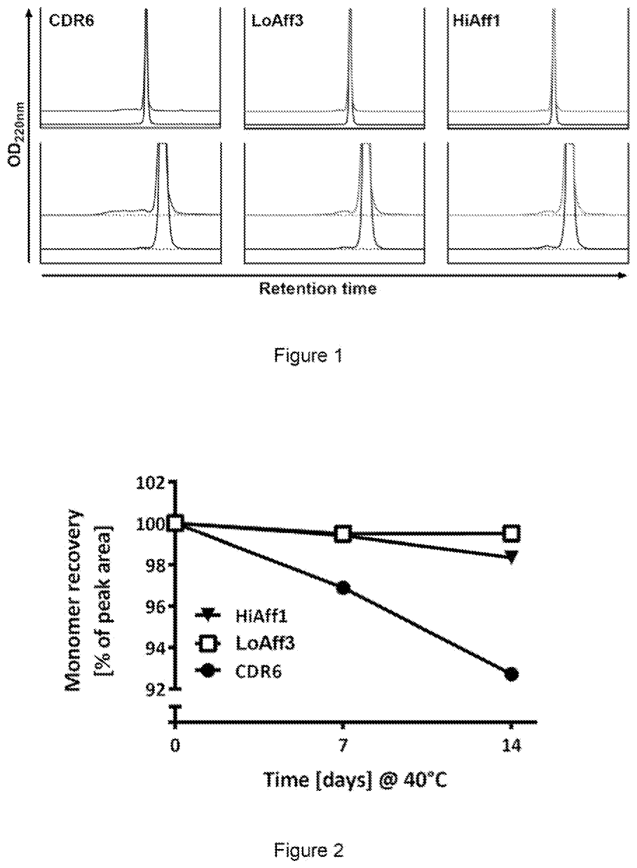

Maturated R16P1C10 TCR variants expressed as soluble bispecific molecules employing a TCR/antiCD3 diabody-F_{c} format. Stress stability testing was performed by incubation of the molecules formulated in PBS for up to two weeks at 40° C. Integrity, aggregate-content (FIG. 1) as well as monomer-recovery (FIG. 2) was analyzed by HPLC-SEC analyses as described above.

### Example 4

### Binding Affinities of Maturated TCR Variants

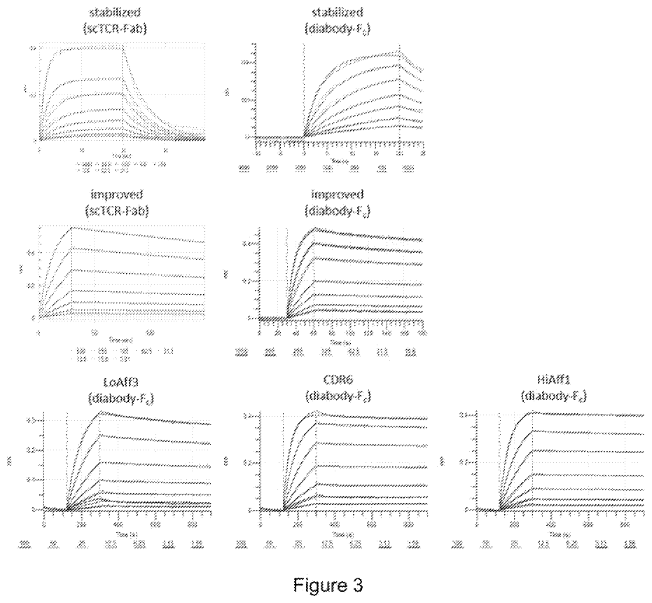

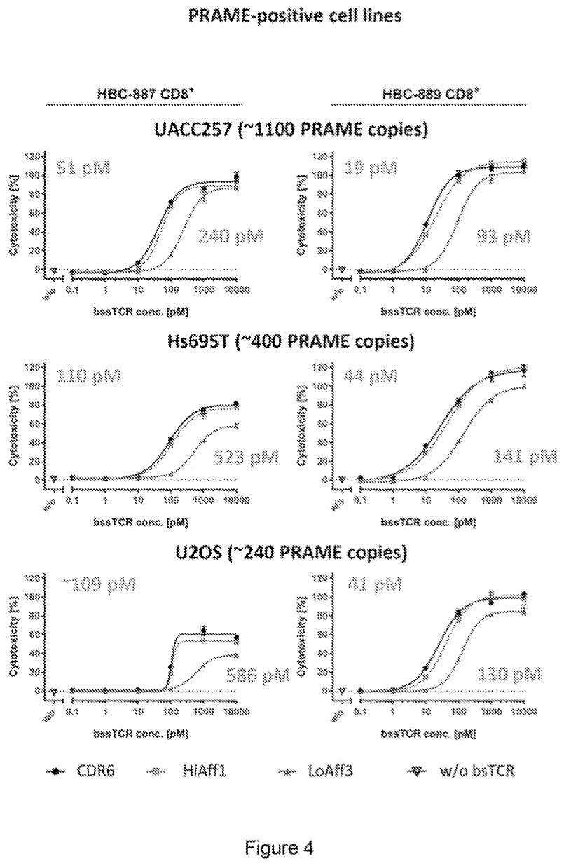

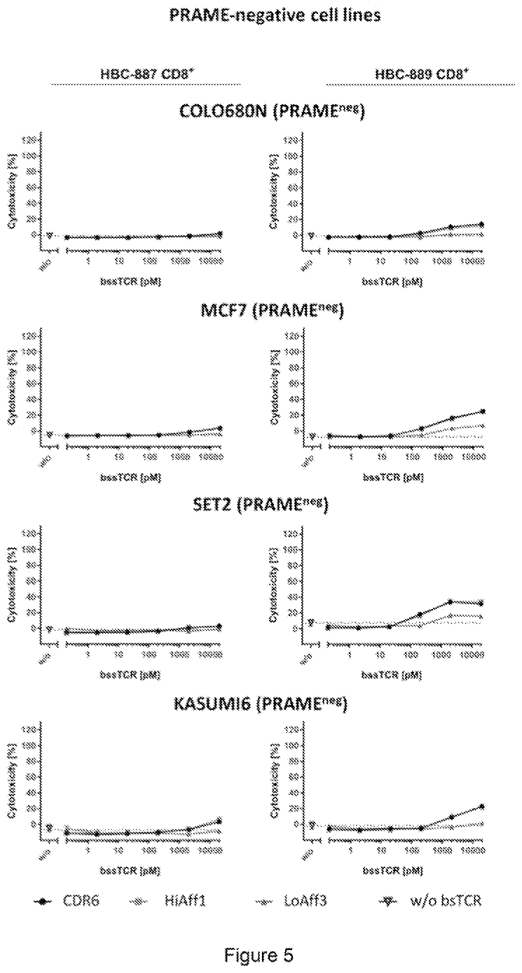

Maturated R16P1C10 TCR variants expressed as soluble bispecific molecules (stabilized, improved: scTCR/antiCD3 Fab format; stabilized, improved, CDR6, HiAff 1 and LoAff3: TCR/antiCD3 diabody-F_{c} format) were analyzed for their binding affinity towards HLA-A*02/PRAME-004 monomers via biolayer interferometry. Measurements were performed on an Octet RED384 system using settings recommended by the manufacturer. Briefly, binding kinetics were measured at 30° C. and 1000 rpm shake speed using PBS, 0.05% Tween-20, 0.1% BSA as buffer. Bispecific molecules were loaded onto biosensors (FAB2G or AHC) prior to analyzing serial dilutions of HLA-A*02/PRAME-004. While a stabilized version of R16P1C10 showed an affinity of approximately 1 μM (1.2 μM as scTCR-Fab, 930 nM as diabody-F_{c}), considerably lower KD values were determined for all variants containing maturated CDRs (Table 3, FIG. 3). To further validate that the affinity of a TCR variant is influenced by the format only to a minor extent, K_{D} values of an affinity-maturated TCR variant were measured as scTCR-Fab or diabody-F_{c} format. The scTCR-Fab and diabody-F_{c} formats showed KD values of 10 nM and 8.7 nM, respectively, further highlighting good comparability between the different formats (Table 3, FIG. 3).

TABLE 3
CDR sequences and binding affinities of wild type and maturated TCRs expressed as scTCR-Fab (based
on SEQ ID NOs: 81 and 82) or diabody-Fc (based on SEQ ID NOs: 119 and 120).

| TCR variant | CDRa1 | CDRa2 | CDRa3 | CDRb1 | CDRb2 | CDRb3 | KD [M] |
|---|---|---|---|---|---|---|---|
| Wild type CDRs | DRGSQS | IYSNGD | CAAVISNFGNEKLTF | SGHRS | YFSETQ | CASSPWDSPNEQYF | Cannot be ex- |
| and framework | | | | | | | pressed in CHO |
| SEQ ID NO: | 11 | 9 | 10 | 12 | 14 | 13 | as scTCR-Fab |
| | | | | | | | or diabody-F_{c} |
| Stabilized¹ | DRGSQS | IYSNGD | CAAVISNFGNEKLTF | PGHRS | YFSETQ | CASSPWDSPNEQYF | 1.2E-06 |
| SEQ ID NO: | 11 | 9 | 10 | 49 | 14 | 13 | |
| Stabilized² | DRGSQS | IYSNGD | CAAVISNFGNEKLTF | PGHRS | YFSETQ | CASSPWDSPNEQYF | 9.3E-07 |
| SEQ ID NO: | 11 | 9 | 10 | 49 | 14 | 13 | |
| Improved¹ | DRGSQS | IYSNGD | CAAVIDNSNGGILTF | PGHRS | YVHGAE | CASSPWDSPNEQYF | 1.0E-08 |
| SEQ ID NO: | 11 | 9 | 19 | 49 | 47 | 13 | |
| Improved² | DRGSQS | IYSNGD | CAAVIDNSNGGILTF | PGHRS | YVHGAE | CASSPWDSPNEQYF | 8.7E-09 |
| SEQ ID NO: | 11 | 9 | 19 | 49 | 47 | 13 | |
| Medium-affinity LoAff3² | DRGSQS | IYQEGD | CAAVIDNDQGGILTF | PGHRS | YVHGEE | CASSPWDSPNEQYF | 1.8E-09 |
| SEQ ID NO: | 11 | 17 | 21 | 49 | 46 | 13 | |
| High-affinity CDR6² | DRGSQS | IYSNGD | CAAVIDNDQGGILTF | PGHRA | YVHGEE | CASSPWDSPNVQYF | 3.9E-10 |
| SEQ ID NO: | 11 | 9 | 21 | 45 | 46 | 48 | |
| High-affinity HiAff1² | DRGSQS | IYQEGD | CAAVIDNDQGGILTF | PGHRA | YVHGEE | CASSPWDSPNVQYF | 3.8E-10 |
| SEQ ID NO: | 11 | 17 | 21 | 45 | 46 | 48 | |

¹expressed as scTCR-Fab
²expressed as diabody-F_{c}

### Example 5

### Killing of Target-Positive and Target-Negative Tumor Cell Lines

Maturated R16P1010 TCR variants were expressed as soluble bispecific molecules employing a TCR/antiCD3 diabody-F_{c} format according to example 1. The cytotoxic activity of the bispecific molecules against PRAME-positive and PRAME-negative tumor cell lines, respectively was analyzed by LDH-release assay. Therefore, tumor cell lines presenting variable amounts of HLA-A*02/PRAME-004 on the cell surface were co-incubated with CD8+ T cells isolated from two healthy donors in presence of increasing concentrations of bispecific molecules. After 48 hours, lysis of target cell lines was measured utilizing CytoTox 96 Non-Radioactive Cytotoxicity Assay Kits (PROMEGA). For all tested PRAME-positive cell lines, highly efficient induction of lysis was detectable and clearly depending on concentration of bispecific molecules. In similar experiments utilizing cell lines expressing HLA-A*02 but not presenting the peptide PRAM E-004 at detectable levels, no or only marginal lysis of targets was induced by the bispecific molecules indicating the specificity of the TCR domains.

### Example 6

### In Vivo Efficacy

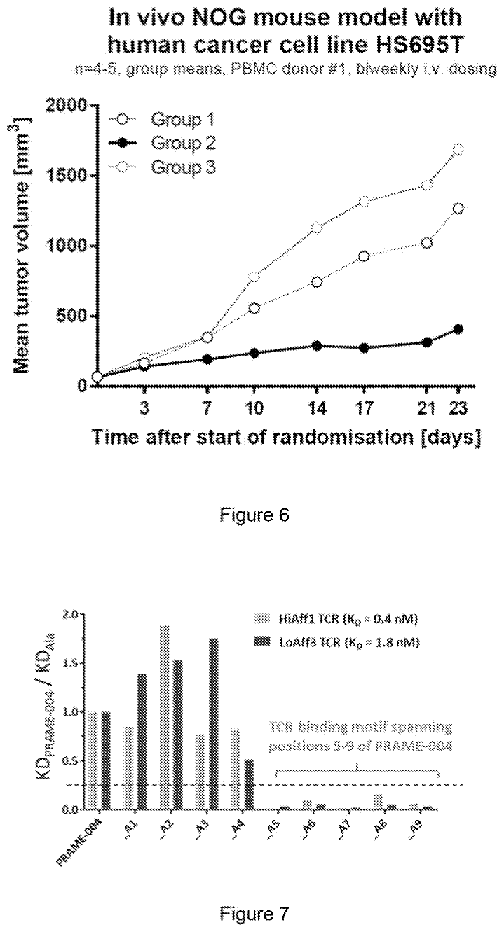

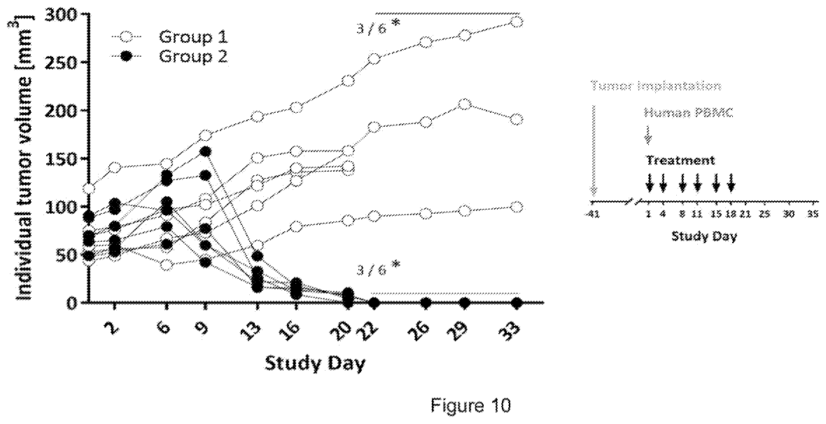

Maturated R16P1010 TCR variant HiAff1 and a HIV-specific high affinity control TCR were expressed as soluble bispecific molecules employing a TCR/antiCD3 diabody-F_{c} format according to example 1 Pharmacodynamic studies designed to test the ability of the bispecific TCR molecules in recruiting and directing the activity of human cytotoxic CD3+ Tcells against PRAME-positive tumor cell lines HS695T and NCI-H1755, as depicted in FIGS. 6 and 10, respectively, were performed in the hyper immune-deficient NOG mouse strain. The NOG mouse strain hosted the subcutaneously injected human tumor cell line HS695T or NCI-H1755 and intravenously injected human peripheral blood mononuclear cell xenografts. Human peripheral blood mononuclear cells (5×10⁶ cells/mouse, intravenous injection) were transplanted within 24 hours when individual tumor volume reached 50 mm³. Treatment was initiated within one hour after transplantation of human blood cells. Four to six female mice per group received intravenous bolus injections (5 mL/kg body weight, twice weekly dosing, up to seven doses, starting one day after randomization) into the tail vein. The injected dose of the PRAM E-targeting bispecific TCR molecule was 0.5 mg/kg body weight per injection (group 2), PBS was used in the vehicle control group (group 1) and the HIV-targeting control TCR bispecific molecule (0.5 mg/kg body weight per injection) in the negative control substance group (group 3). At the indicated time points, mean tumor volumes were calculated for every group based on the individual tumor volumes that were measured with a caliper and calculated as length×width²/2. Treatment with PRAME-targeting bispecific TCR molecule inhibited HS695T tumor growth as indicated by reduced increase of tumor volume from basal levels (start of randomization) of 65 to 409 mm³ in comparison to the increase observed in the vehicle control group from basal levels of 69 to 1266 mm³ and the negative control substance group from basal levels of 66 to 1686 mm³ at day 23 (FIG. 6). Treatment with PRAME-targeting bispecific TCR molecule induced a complete remission of NCI-H1755 tumors. Tumors growth was inhibited in all animals and tumors were not detectable in remaining animals at Day 22 (basal mean levels at start of randomization 68 mm³) until the end of the study at Day29 (Group 2). In the vehicle treated group (Group 1) an ongoing increase of the tumor volume was observed from basal mean levels of 70 to 175 mm³ at Day 22 (FIG. 10).

### Example 7

### Off-Target Peptide Identification and Confirmation

Bispecific TCR molecules incorporating the maturated CDR6, HiAff1 and LoAff3 TCR variants of R16P1010 were designed as TCR/antiCD3 diabody-F_{c} format according to Example 1. For identification of potential off-target peptides of maturated TCR variants, the PRAME-004 residues involved in TCR binding were identified via binding affinity measurements against PRAME-004 peptides by mutational analysis (FIG. 7, _A1 to _A9). Affinity measurements were performed according to example 4 but using SA sensors to load biotinylated HLA-A*02/peptide complexes (at a concentration of 1 μg/ml for 180 s) and analyze bispecific molecules in solution. Positions 5-9 of PRAME-004 were identified as relevant for TCR binding based on greater than 4-fold decrease in binding affinity of the variants of PRAME-004 (indicated as dotted line). Potential off-target peptides were identified via blast search against a database of normal tissue-presented HLA-A*02 bound peptides (XPRESIDENT database) using a similarity scoring within the binding-relevant positions 5-9 of PRAME-004. 27 different potential off-targets were identified by similarity BLAST search (Table 4). To test the relevance of the potential off-targets, the peptides were loaded onto HLA-A*02-positive T2 target cells at a concentration of 10 nM. Peptide-loaded T2 cells were co-incubated with human CD8⁺ Tcells of two different donors at an effector/target ratio of 5:1 in the presence of increasing concentrations of bispecific TCR molecules. Out of 27 potential off-targets, the peptides IFT17-003, IFIT1-001, FADS2-001, CTBP1-001, ATP1A1-001, NADK-002 and ITSN1-001 induced detectable cytotoxicity against peptide-loaded T2 cells, respectively. Based on the determined EC50 values of cytotoxicity, the off-target peptides IFT17-003, IFIT1-001, FADS2-001 and CTBP1-001 were confirmed as the most relevant off-targets (Table 4).

TABLE 4

| | | SEQ | CDR6; EC50 [Pm] | | HiAff1; EC50 [pM] | | LoAff3; EC50 [pM] | |
|---|---|---|---|---|---|---|---|---|
| Peptide | Peptide | ID | Donor | Donor | Donor | Donor | Donor | Donor |
| code | sequence | NO | 1 | 2 | 1 | 2 | 1 | 2 |
| PRAME-004 | SLLQHLIGL | 8 | 1.0 | 0.3 | 1.2 | 1.1 | 6.6 | 2.5 |
| IFT17-003 | FMNPHLISV | 51 | 1.6 | 1.5 | 1.6 | 0.3 | 2.6 | 3.0 |
| IFIT1-001 | VLLHHQIGL | 50 | 38 | 97 | 38 | 21 | 522 | 269 |
| FADS2-001 | LLLAHIIAL | 52 | 112 | 100 | 84 | 82 | 447 | 1027 |
| CTBP1-001 | ALMYHTITL | 53 | 115 | 66 | 79 | 44 | 2167 | 920 |
| ATP1A1-001 | FLPIHLLGL | 125 | 201 | 375 | 196 | 437 | 292 | — |
| NADK-002 | YLDGHUTT | 126 | 229 | 496 | 202 | 2034 | 277 | — |
| ITSN1-001 | ILAMHLIDV | 127 | 1960 | 1915 | 1024 | 1015 | 245 | — |
| SMA-003 | IVINHVISV | 128 | — | — | — | — | — | — |
| VPS13A-001 | ILQPHVIAL | 129 | — | — | — | — | — | — |
| SF3B3-005 | TLVYHVVGV | 130 | — | — | — | — | — | — |
| NCAM2-001 | FVQPHIIQL | 131 | — | — | — | — | — | — |
| KNT-001 | YMLEHVITL | 132 | — | — | — | — | — | — |
| EHD4-001 | ALAKHLIKI | 133 | — | — | — | — | — | — |
| EHD-001 | ALANHLIKV | 134 | — | — | — | — | — | — |
| MCMB-002 | YLILHLIST | 135 | — | — | — | — | — | — |
| HSPA5-001 | RVMEHFIKL | 136 | — | — | — | — | — | — |
| CEBPZ-002 | KLYQHEINL | 137 | — | — | — | — | — | — |
| DCAF12-001 | SFYEHIITV | 138 | — | — | — | — | — | — |
| EHD2-002 | ALASHLIEA | 139 | — | — | — | — | — | — |
| KIAA1324-001 | SLADRLIGV | 140 | — | — | — | — | — | — |
| MYO1A-001 | LVSEHVIEL | 141 | — | — | — | — | — | — |
| SERPINA6-001 | SLYKHLVAL | 142 | — | — | — | — | — | — |
| SMARCD1- | IINHVISV | 143 | — | — | — | — | — | — |
| 001 | | | | | | | | |
| | | SEQ | CDR6; EC50 [pM] | | HiAff1; EC50 [pM] | | LoAff3; EC50 [pM] | |
| Peptide | Peptide | ID | Donor | Donor | Donor | Donor | Donor | Donor |
| code | sequence | NO: | 1 | 2 | 1 | 2 | 1 | 2 |
| TSC1-001 | SLLGHVIRL | 144 | — | — | — | — | — | — |
| UGT-005 | SLIEHMIML | 145 | — | — | — | — | — | — |
| VAV1-001 | NLLPHAINL | 146 | — | — | — | — | — | — |
| WDFY3-004 | SLFEHFIEL | 147 | — | — | — | — | — | — |

### Example 8

### Specificity Maturation

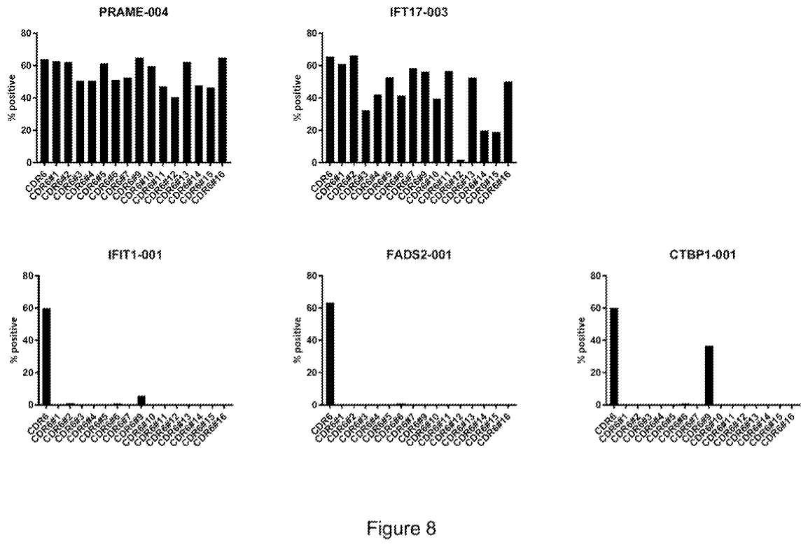

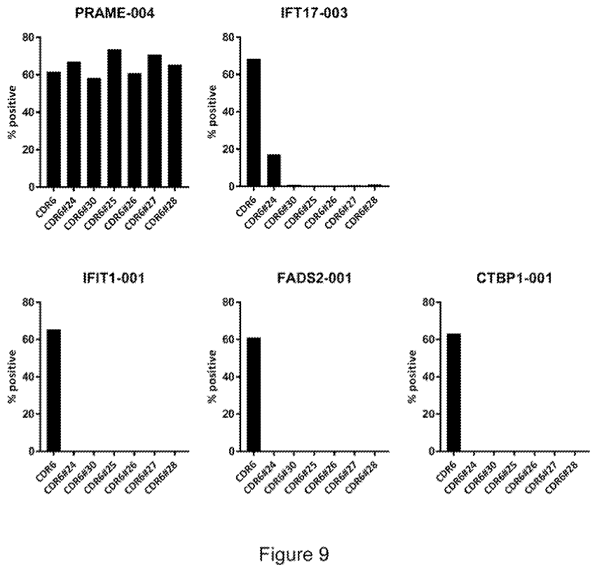

In order to increase the cytotoxic window between PRAME-004 and healthy tissue expressed off-target peptides IFT17-003, IFIT1-001, FADS2-001 and CTBP1-001, yeast surface display was performed, based on the TCR variant CDR6. For this, the according sequence was converted into the scTCR CDR6 (SEQ ID NO: 150) with an appropriate glycine-serine linker sequence (SEQ ID NO: 151). The DNA of the corresponding sequence was synthesized with a degenerate CDRa3 and transformed into Saccharomyces cerevisiae EBY100 (MATa AGA1::GAL1AGA1::URA3 ura352 trp1 leu2delta200 his3delta200 pep4::HIS3 prbd1.6R can1 GAL) (ATCC® MYA 4941TM) together with a yeast display vector containing a leader sequence and the Aga2p yeast mating protein (SEQ ID NO: 152), based on pCT302 (Boder et al. Methods Enzymol. 2000; 328: 430-44). The resulting fusion protein after homologous recombination in the yeast (SEQ ID NO: 153) contains a leader peptide at the N-terminus of the Aga2p protein, responsible for the display of the protein of interest (Boder et al., Nat Biotechnol. 1997 Jun; 15(6): 553-7), short peptide tags (FLAG and Myc) including linker sequences (SEQ ID NOs: 154 and 84, respectively) for expression controls and the protein of interest, namely the scTCR CDR6 (SEQ ID NO: 150) or its variants. The transformation was performed as described in DE102016121899 and resulted in up to 109 yeast clones per library. Yeast surface display and the according selection and analysis were performed essentially as described in Smith et al. 2015 (Mol Biol. 2015; 1319:95-141). For the selection of specificity-improved, but still highly affine scTCR variants, a low concentration of monomeric HLA-A*02/PRAME-004 was used for positive selection. Simultaneously, negative selection against the 4 most relevant off-target peptides, namely IFIT1-001, I FT17-003, FADS2-001 and CTBP1-001 was performed in the context of HLA-A*02. After 3 selection rounds, single clones were isolated and analyzed (FIG. 8) and the remaining bulk selection was subjected to an error-prone PCR over the whole scTCR sequence, using the GeneMorph II (Agilent) according to manufacturer's protocol. The resulting DNA was transformed into yeast as described above in order to generate a new selection library. With this newly derived library, the selection process for high affinity towards HLA-A*02/PRAME-004 and minimal binding to HLA-A*02/off-target peptides was performed as described above and resulted in variants with strongly increased discrimination between binding to PRAME-004 and the off-target peptides IFIT1-001, IFT17-003, FADS2-001 and CTBP1-001 (FIG. 9). scTCR variants derived from an error-prone PCR library of a CDRa3 library originating from scTCR CDR6 were displayed on the yeast surface and analyzed for binding towards PRAME-004 and the off-target peptides IFT17-003, IFIT1-001, FADS2-001 and CTBP1-001 in the context of HLA-A*02, respectively. All HLA-A*02/peptide complexes were added to the scTCR displaying yeast at a concentration of 100 nM and the percentage of positive yeast cells was determined via flow cytometry analysis.

### Example 9

### In Vitro Safety Assessment with Healthy Tissue Cells

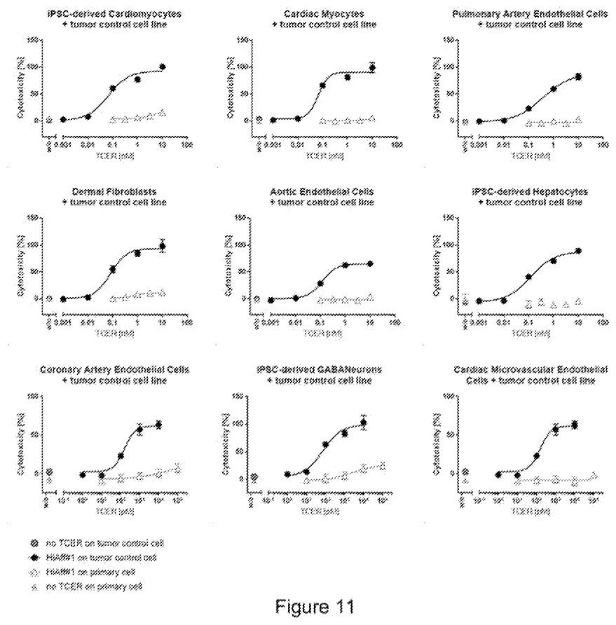

Maturated R16P1C10 TCR variant HiAff1 was expressed as soluble bispecific molecules employing a TCR/antiCD3 diabody-F_{c} format according to example 1. The safety profile of TCER® molecule HiAff1 was assessed in LDH-killing experiments with human normal tissue primary cells of different organs (FIG. 11). PBMC effector cells from healthy HLA-A*02+ donors were co-cultured with primary tissue cells at a ratio of 10:1 in the presence of increasing TCER® concentrations. The cells were co-incubated in a 1:1 mixture of normal tissue primary cell-specific medium and T cell medium. As control the PRAME-positive tumor cell line Hs695T was used in the respective medium combination of the normal tissue primary cells. After 48 h of co-culture, supernatants were harvested and tumor cell lysis was analyzed by measuring LDH-release using the LDH-Glo™ Kit (Promega). Lysis was calculated based on LDH release, with total LDH content in target cells alone serving as 100% lysis value.

### Example 10

### Safety Evaluated in Whole Blood

A bispecific TCR molecule incorporating the maturated HiAff 1 TCR variant of R16P1010 was designed as TCR/antiCD3 diabody-Fc format according to Example 1. Cytokine release induced by HiAff 1 TCER® was assessed in vitro in whole blood (assays were performed by HOT Screen GmbH, Reutlingen, Germany). Whole blood freshly obtained from three healthy HLA-A*02-positive donors was incubated with defined concentrations of HiAff1 or anti-CD3 control antibody in the presence of human umbilical vein endothelial cells (HUVEC; 10,000 cells/well). After 48 h supernatants were collected and stored at −20° C. prior to determination of cytokine concentrations in the supernatant by multiplexed analysis (Luminex®-based technology). Exemplary data of IFN-γ and IL-6 release highlight the good safety profile of HiAff1 TCER® showing no to minimal cytokine release compared to anti-CD3 control antibody (FIG. 12).

### Example 11

### Detection of PRAME Peptide on Primary Tissues by Mass Spectrometry

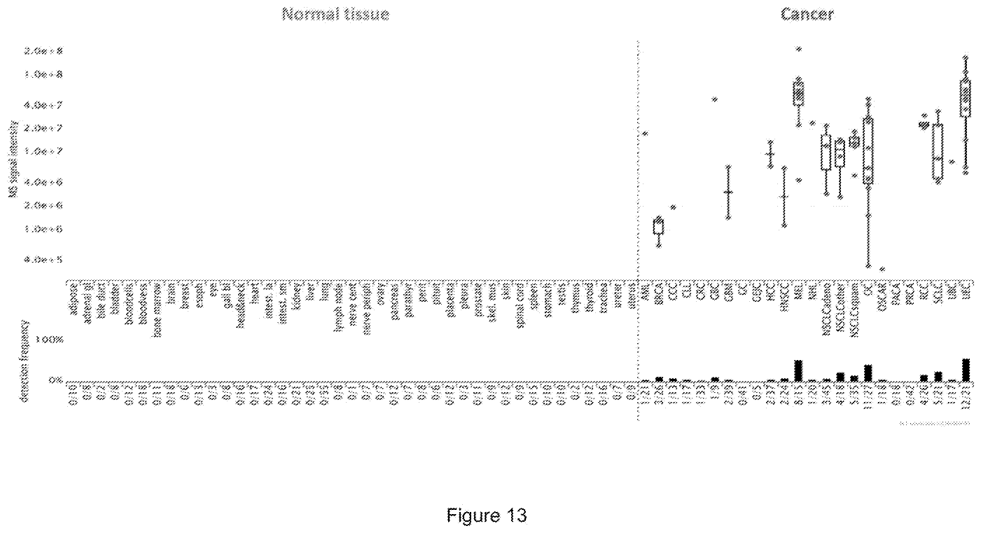

For the identification and relative quantitation of HLA ligands by mass spectrometry, HLA molecules from shock-frozen tissue samples were purified and HLA-associated peptides were isolated. The isolated peptides were separated, and sequences were identified by online nano-electrospray-ionization (nanoESl) liquid chromatography-mass spectrometry (LC-MS) experiments. Since the peptides were directly identified as ligands of HLA molecules of primary tumors, these results provide direct evidence for the natural processing and presentation of the identified peptides on the primary cancer tissue, and hence the LC-MS signal areas of a peptide correlate with its abundance in the sample. The acquired LC-MS data are subsequently processed and quantified using a proprietary label-free quantitation data analysis pipeline, combining algorithms for sequence identification, spectral clustering, ion counting, retention time alignment, charge state deconvolution and normalization (see FIG. 13).

1. An antigen binding protein which specifically binds to a PRAME peptide, which PRAME peptide comprises the amino acid sequence SLLQHLIGL of SEQ ID NO: 8 and is in a complex with a MHC protein, the antigen binding protein comprising
(a) a first polypeptide chain comprising a first variable domain comprising three complementary determining regions (CDRs) CDRa1, CDRa2 and CDRa3, wherein
the CDRa1 comprises or consists of the amino acid sequence DRGSQX₁ (SEQ ID NO: 1), wherein X₁ is any amino acid,
the CDRa2 comprises or consists of the amino acid sequence IYX₂X₃GD (SEQ ID NO: 2), wherein X₂ and X₃ is any amino acid, with the proviso that the CDRa2 does not comprise or consist of the amino acid sequence IYSNGD (SEQ ID NO: 9),
the CDRa3 comprises or consists of the amino acid sequence CAAVIX₄NX₅X₆GGX₇LTF (SEQ ID NO: 3), wherein X₄ to X₇ is any amino acid,
and
(b) a second polypeptide chain comprising a second variable domain comprising three complementary determining regions (CDRs) CDRb1, CDRb2 and CDRb3, wherein
the CDRb1 comprises or consists of the amino acid sequence X₈GHRX₉ (SEQ ID NO: 4), wherein X₈ and X₉ is any amino acid, and
the CDRb2 comprises or consists of the amino acid sequence YX₁₀X₁₁X₁₂X₁₃X₁₄ (SEQ ID NO: 5), wherein X₁₀ to X₁₄ is any amino acid, and
the CDRb3 comprises or consists of the amino acid sequence CASSPWDSPNX₁₅QYF (SEQ ID NO: 6), wherein X₁₅ is any amino acid.

2. An antigen binding protein of claim 1, wherein said antigen binding protein specifically binds to the peptide comprising the amino acid sequence of SEQ ID NO: 8 of PRAME in a complex with a MHC protein, optionally a HLA protein.

3. The antigen binding protein of claim 1, wherein said antigen binding protein binds to a complex of said PRAME peptide and HLA-A*02 with a K_{D} which is ≤200 nM.

4. The antigen binding protein of claim 1, wherein the antigen binding protein is an antibody or a fragment thereof, or a bispecific antibody or a fragment thereof, or a T cell receptor (TCR) or fragment thereof, or a bispecific T cell receptor or a fragment thereof.

5. The antigen binding protein of claim 1, wherein the first polypeptide and the second polypeptide are linked together.

6. The antigen binding protein of claim 5, wherein said antigen binding protein is a single chain TCR (scTCR) or a single-chain bispecific antibody.

7. The antigen binding protein of claim 1, wherein said first variable domain further comprises one or more framework regions selected from the group consisting of FR1-a, FR2-a, FR3-a and FR4-a, wherein
FR1-a comprises or consists of the amino acid sequence of Q SEQ ID NO: 54 or an amino acid sequence at least 85% identical to SEQ ID NO: 54,
FR2-a comprises or consists of the amino acid sequence of SEQ ID NO: 55 or an amino acid sequence at least 85% identical to SEQ ID NO: 55,
FR3-a comprises or consists of the amino acid sequence of SEQ ID NO: 56 or an amino acid sequence at least 85% identical to SEQ ID NO: 56,
FR4-a comprises or consists of the amino acid sequence of SEQ ID NO: 57 or an amino acid sequence at least 85% identical to SEQ ID NO: 57, and
said second variable domain further comprises one or more framework regions selected from the group consisting of FR1-b, FR2-b, FR3-b and FR4-b, wherein
FR1-b comprises or consists of the amino acid sequence of SEQ ID NO: 58 or an amino acid sequence at least 85% identical to SEQ ID NO: 58,
FR2-b comprises or consists of the amino acid sequence of SEQ ID NO: 59 or an amino acid sequence at least 85% identical to SEQ ID NO: 59,
FR3-b comprises or consists of the amino acid sequence of SEQ ID NO: 60 or an amino acid sequence at least 85% identical to SEQ ID NO: 60, or
FR3-b comprises or consists of the amino acid sequence of SEQ ID NO: 61 or an amino acid sequence at least 85% identical to SEQ ID NO: 61, or
FR4-b comprises or consists of the amino acid sequence of SEQ ID NO: 62 or an amino acid sequence at least 85% identical to SEQ ID NO: 62.

8. The antigen binding protein of claim 1, wherein said antigen binding protein comprises
(i) a first polypeptide chain comprising a first variable domain comprising the amino acid sequence selected from the group consisting of, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99 and SEQ ID NO: 100 or an amino acid sequence at least 85% identical to the amino acid sequence selected from the group consisting of, SEQ ID NO: 85, SEQ ID NO: 86, SEQ ID NO:87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, and
a second polypeptide chain comprising a second variable domain comprising the amino acid sequence of SEQ ID NO: 83 or an amino acid sequence at least 85%, at least 90% or at least 95% identical to SEQ ID NO: 83, or
(ii) a first polypeptide chain comprising a first variable domain comprising the amino acid sequence selected from the group consisting of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111 or an amino acid sequence at least 85%, at least 90% or at least 95% identical to the amino acid sequence selected from the group consisting of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, and a second polypeptide chain comprising a second variable domain comprising the amino acid sequence of SEQ ID NO: 83 or an amino acid sequence at least 85%, at least 90% or at least 95% identical to SEQ ID NO: 83, or
(iii) a first polypeptide chain comprising a first variable domain comprising the amino acid sequence selected from the group consisting of SEQ ID NO: 113, SEQ ID NO: 114 SEQ ID NO: 115 SEQ ID NO: 116 SEQ ID NO: 63 SEQ ID NO: 118 or an amino acid sequence at least 85%, at least 90% or at least 95% identical to the amino acid sequence selected from the group consisting of SEQ ID NO: 113, SEQ ID NO: 114 SEQ ID NO: 115 SEQ ID NO: 63 SEQ ID NO: 63 SEQ ID NO: 118, and
a second polypeptide chain comprising a second variable domain comprising the amino acid sequence of SEQ ID NO: 112 or an amino acid sequence at least 85%, at least 90% or at least 95% identical to SEQ ID NO: 112.

9. The antigen binding protein of claim 1, wherein said antigen binding protein comprises
(i) a first polypeptide chain comprising a first variable domain comprising the amino acid sequence selected from the group consisting of, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO:96, SEQ ID NO: 97, SEQ ID NO: 98, SEQ ID NO: 99 and SEQ ID NO: 100 or variant thereof comprising or consisting of an amino acid sequence at least 85%, at least 90% or at least 95% identical to the amino acid sequence selected from the group consisting of, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, and wherein the CDRs in said first variable domain or variant thereof optionally comprises the amino acid sequence of CDRa1 of SEQ ID NO: 11, CDRa2 of SEQ ID NO: 17 and CDRa3 of SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 32, SEQ ID NO: 33, SEQ ID NO: 34, SEQ ID NO: 35, SEQ ID NO: 36, SEQ ID NO: 38, SEQ ID NO: 181 or SEQ ID NO: 182, optionally comprising one or two amino acid substitutions, and
a second polypeptide chain comprising a second variable domain comprising the amino acid sequence of SEQ ID NO: 83 or a variant thereof comprising or consisting of an amino acid sequence at least 85%, at least 90% or at least 95% identical to SEQ ID NO: 83, and wherein the CDRs in said second variable domain or variant thereof preferably comprises the amino acid sequence of CDRb1 of SEQ ID NO: 45, CDRb2 of SEQ ID NO: 46 and CDRb3 of SEQ ID NO: 48, optionally comprising one or two amino acid substitutions, or
(ii) a first polypeptide chain comprising a first variable domain comprising the amino acid sequence selected from the group consisting of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, or SEQ ID NO: 180 or a variant thereof comprising or consisting of an amino acid sequence at least 85%, at least 90% or at least 95% identical to the amino acid sequence selected from the group consisting of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111 or SEQ ID NO: 180 and wherein the CDRs in said first variable domain or variant thereof optionally comprises the amino acid sequence of CDRa1 of SEQ ID NO: 16, CDRa2 of SEQ ID NO: 17 and CDRa3 of SEQ ID NO: 38, SEQ ID NO: 24, SEQ ID NO: 26, SEQ ID NO: 33, SEQ ID NO: 39, SEQ ID NO: 40, SEQ ID NO: 42, SEQ ID NO: 43, SEQ ID NO: 179 SEQ ID NO: 44, SEQ ID NO: 21, optionally comprising one or two amino acid substitutions, and
a second polypeptide chain comprising a second variable domain comprising the amino acid sequence of SEQ ID NO: 83 or a variant thereof comprising or consisting of an amino acid sequence at least 85%, at least 90% or at least 95% identical to SEQ ID NO: 83, and wherein the CDRs of said second variable domain or variant thereof optionally comprises the amino acid sequence of CDRb1 of SEQ ID NO: 45, CDRb2 of SEQ ID NO: 46 and CDRb3 of SEQ ID NO: 48, optionally comprising one or two amino acid substitutions, or
(iii) a first polypeptide chain comprising a first variable domain comprising the amino acid sequence selected from the group consisting of SEQ ID NO: 113, SEQ ID NO: 114 SEQ ID NO: 115 SEQ ID NO: 116 SEQ ID NO: 63 SEQ ID NO: 118 or a variant thereof comprising or consisting of an amino acid sequence at least 85%, at least 90% or at least 95% identical to the amino acid sequence selected from the group consisting of SEQ ID NO: 113, SEQ ID NO: 114 SEQ ID NO: 115 SEQ ID NO: 63 SEQ ID NO: 63 SEQ ID NO: 118, and wherein the CDRs of said first variable domain or variant thereof optionally comprises the amino acid sequence of CDRa1 of SEQ ID NO: 16 or SEQ ID NO: 11, CDRa2 of SEQ ID NO: 17 and CDRa3 of SEQ ID NO: 40, SEQ ID NO: 38, SEQ ID NO: 39, SEQ ID NO: 26 or SEQ ID NO: 21, optionally comprising one or two amino acid substitutions, and
a second polypeptide chain comprising a second variable domain comprising the amino acid sequence of SEQ ID NO: 112 or a variant thereof comprising or consisting of an amino acid sequence at least 85%, at least 90% or at least 95% identical to SEQ ID NO: 112, and wherein the CDRs of said second variable domain or variant thereof optionally comprises the amino acid sequence of CDRb1 of SEQ ID NO: 45, CDRb2 of SEQ ID NO: 46 and CDRb3 of SEQ ID NO: 48, optionally comprising one or two amino acid substitutions.

10. The antigen binding protein of claim 1, wherein said first variable domain is comprised in a TCR α or γ chain; and/or wherein said second variable domain is comprised in a TCR β or δ chain.

11. The antigen binding protein of claim 1, further comprising one or more of the following:
(i) one or more further antigen binding sites;
(ii) a transmembrane region, optionally including a cytoplasmic signalling region;
(iii) a diagnostic agent;
(iv) a therapeutic agent; or
(v) PK modifying moiety.

12. The antigen binding protein of claim 1, comprising two polypeptide chains that form two antigen binding sites, wherein a first polypeptide chain has a structure represented by formula:
V₃-L₁-V₄-L₂-C_{L} [I]
wherein V₃ is a third variable domain;
V₄ is a fourth variable domain;
L₁ and L₂ are linkers;
L₂ may be present or absent;
C_{L} is a light chain constant domain or a portion thereof and present or absent;
and wherein a second polypeptide chain has a structure represented by formula:
V₅-L₃-V₆-L₄-C_{H1} [II]
wherein V₅ is a fifth variable domain;
V₆ is a sixth variable domain;
L₃ and L₄ are linkers;
L₄ may be present or absent;
C_{H1} is a heavy chain constant domain 1 or a portion thereof and is present or absent;
and wherein
V₃ or V₄ is a first variable domain
V₅ or V₆ is a second variable domain as defined in claim 1, or
V₅ or V₆ is a first variable domain as defined in claim 1 and V₃ or V₄ is a second variable domain as defined in claim 1, and wherein
when V₃ and V₅ are variable domains as defined in claim 1, one of V₄ or V₆ is a light chain variable domain and the other is a heavy chain variable domain, and wherein
when V₃ and V₆ are variable domains as defined in claim 1, one of V₄ or V₅ is a light chain variable domain and the other is a heavy chain variable domain and wherein the light chain variable domain and the heavy chain variable domain form together one antigen binding site.

13. The antigen binding protein of claim 12, wherein said heavy chain variable domain and said light chain variable domain bind to an antigen selected from the group consisting of and the light chain variable domain (V_{L}) bind to an antigen selected from the group consisting of CD3 (such as the CD3γ, CD3δ, and CD3ε chains), CD4, CD7, CD8, CD10, CD11b, CD11c, CD14, CD16, CD18, CD22, CD25, CD28, CD32a, CD32b, CD33, CD41, CD41b, CD42a, CD42b, CD44, CD45RA, CD49, CD55, CD56, CD61, CD64, CD68, CD94, CD90, CD117, CD123, CD125, CD134, CD137, CD152, CD163, CD193, CD203c, CD235a, CD278, CD279, CD287, Nkp46, NKG2D, GITR, F_{c}εRI, TCRα/β and TCRγ/δ, HLA-DR and/or bind to an effector cell.

14. The antigen binding protein of claim 12, comprising two polypeptide chains that form two antigen-binding sites, wherein one polypeptide chain has a structure represented by formula [III]:
V₃-L₁-V₄-L₂-C_{L}-L₅-F_{c1} [III]
and one polypeptide chain has a structure represented by formula [IV]:
V₅-L₃-V₆-L₄-C₁-L₆-F_{c2} [IV]
wherein L₅ and L₆ are linkers and present or absent and F_{c1}, and F_{c2} are F_{c}-domains and wherein F_{c1} and F_{c2} are the same or different.

15. An isolated nucleic acid comprising a sequence encoding an antigen binding protein of claim 1.

16. A vector comprising the nucleic acid of claim 15.

17. A host cell which has been transformed with a nucleic acid of claim 15 or a vector comprising the nucleic acid.

18. A host cell comprising the antigen binding protein of claim 1, or an isolated nucleic acid comprising a sequence encoding the antigen binding protein, or a vector comprising the nucleic acid.

19. A pharmaceutical composition comprising the antigen binding protein of claim 1, an isolated nucleic acid comprising a sequence encoding the antigen binding protein, a vector comprising the nucleic acid, or a host cell which has been transformed with the isolated nucleic acid or a vector comprising the nucleic acid and a pharmaceutically acceptable carrier.

20. A method of producing the antigen binding protein according to claim 1, comprising
1. providing a host cell,
2. providing a genetic construct comprising a coding sequence encoding the antigen binding protein of claim 1,
3. introducing said genetic construct into said host cell, and
4. expressing said genetic construct by said host cell.

21. The method of claim 20, further comprising isolation and purification of the antigen binding protein from the host cell and, optionally, reconstitution of the antigen binding protein in a T-cell.

22. The antigen binding protein of claim 1, an isolated nucleic acid comprising a sequence encoding the antigen binding protein, a vector comprising the nucleic acid, a host cell comprising the antigen binding protein or an isolated nucleic acid comprising a sequence encoding the antigen binding protein, or a vector comprising the nucleic acid, or a pharmaceutical composition thereof, adapted for use in medicine.

23. The antigen binding protein of claim 1, a nucleic acid comprising a sequence encoding the antigen binding protein, a vector comprising the nucleic acid, a host cell comprising the antigen binding protein or an isolated nucleic acid comprising a sequence encoding the antigen binding protein, or a vector comprising the nucleic acid, or the pharmaceutical composition thereof, adapted for use in diagnosis, prevention, and/or treatment of a proliferative disease, optionally cancer, wherein said cancer is selected from the group of cancers consisting of lung cancer, optionally non-small cell lung cancer, small cell lung cancer, liver cancer, head and neck cancer, skin cancer, renal cell cancer, brain cancer, gastric cancer, colorectal cancer, hepatocellular cancer, pancreatic cancer, prostate cancer, leukemia, breast cancer, Merkel cell carcinoma, melanoma, ovarian cancer, urinary bladder cancer, uterine cancer, gallbladder and bile duct cancer, and esophageal cancer.
